SEQUENCE LISTING (159 sequences)

SEQ ID NO: 1 - protein, 824 aa, Homo sapiens
MRQSLLFLTSVVPFVLAPRPPDDPGFGPHQRLEKLDSLLSDYDILSLSNIQQHSVRKRDLQTSTHVETLLTFSALKRHFKLYLTSSTERFSQNFKVVVVDGKNESEYTVKWQDFFTGHVVGEPDSRVLAHIRDDDVIIRINTDGAEYNIEPLWRFVNDTKDKRMLVYKSEDIKNVSRLQSPKVCGYLKVDNEELLPKGLVDREPPEELVHRVKRRADPDPMKNTCKLLVVADHRFYRYMGRGEESTTTNYLIELIDRVDDIYRNTSWDNAGFKGYGIQIEQIRILKSPQEVKPGEKHYNMAKSYPNEEKDAWDVKMLLEQFSFDIAEEASKVCLAHLFTYQDFDMGTLGLAYVGSPRANSHGGVCPKAYYSPVGKKNIYLNSGLTSTKNYGKTILTKEADLVTTHELGHNFGAEHDPDGLAECAPNEDQGGKYVMYPIAVSGDHENNKMFSNCSKQSIYKTIESKAQECFQERSNKVCGNSRVDEGEECDPGIMYLNNDTCCNSDCTLKEGVQCSDRNSPCCKNCQFETAQKKCQEAINATCKGVSYCTGNSSECPPPGNAEDDTVCLDLGKCKDGKCIPFCEREQQLESCACNETDNSCKVCCRDLSGRCVPYVDAEQKNLFLRKGKPCTVGFCDMNGKCEKRVQDVIERFWDFIDQLSINTFGKFLADNIVGSVLVFSLIFWIPFSILVHCVDKKLDKQYESLSLFHPSNVEMLSSMDSASVRIIKPFPAPQTPGRLQPAPVIPSAPAAPKLDHQRMDTIQEDPSTDSHMDEDGFEKDPFPNSSTAAKSFEDLTDHPVTRSEKAASFKLQRQNRVDSKETEC

SEQ ID NO: 2 - protein, 698 aa, Homo sapiens
MRQSLLFLTSVVPFVLAPRPPDDPGFGPHQRLEKLDSLLSDYDILSLSNIQQHSVRKRDLQTSTHVETLLTFSALKRHFKLYLTSSTERFSQNFKVVVVDGKNESEYTVKWQDFFTGHVVGEPDSRVLAHIRDDDVIIRINTDGAEYNIEPLWRFVNDTKDKRMLVYKSEDIKNVSRLQSPKVCGYLKVDNEELLPKGLVDREPPEELVHRVKRRADPDPMKNTCKLLVVADHRFYRYMGRGEESTTTNYLIELIDRVDDIYRNTSWDNAGFKGYGIQIEQIRILKSPQEVKPGEKHYNMAKSYPNEEKDAWDVKMLLEQFSFDIAEEASKVCLAHLFTYQDFDMGTLGLAYVGSPRANSHGGVCPKAYYSPVGKKNIYLNSGLTSTKNYGKTILTKEADLVTTHELGHNFGAEHDPDGLAECAPNEDQGGKYVMYPIAVSGDHENNKMFSNCSKQSIYKTIESKAQECFQERSNKVCGNSRVDEGEECDPGIMYLNNDTCCNSDCTLKEGVQCSDRNSPCCKNCQFETAQKKCQEAINATCKGVSYCTGNSSECPPPGNAEDDTVCLDLGKCKDGKCIPFCEREQQLESCACNETDNSCKVCCRDLSGRCVPYVDAEQKNLFLRKGKPCTVGFCDMNGKCEKRVQDVIERFWDFIDQLSINTFGKFLADNIVGSVLVFSLIFWIPFSILVHCVTSKC

SEQ ID NO: 3 - protein, 862 aa, Mus musculus
MSTADLMRRWVIALLLAAAGVAAEDSCSRNEFQCRDGKCIASKWVCDGSPECPDGSDESPETCMSVTCQSNQFSCGGRVSRCIPDSWRCDGQVDCENDSDEQGCPPKTCSQDDFRCQDGKCISPQFVCDGDRDCLDGSDEAHCQATTCGPAHFRCNSSICIPSLWACDGDVDCVDGSDEWPQNCQGRDTASKGVSSPCSSLEFHCGSSECIHRSWVCDGEADCKDKSDEEHCAVATCRPDEFQCADGSCIHGSRQCDREHDCKDMSDELGCVNVTQCDGPNKFKCHSGECISLDKVCDSARDCQDWSDEPIKECKTNECLDNNGGCSHICKDLKIGSECLCPSGFRLVDLHRCEDIDECQEPDTCSQLCVNLEGSYKCECQAGFHMDPHTRVCKAVGSIGYLLFTNRHEVRKMTLDRSEYTSLLPNLKNVVALDTEVTNNRIYWSDLSQKKIYSALMDQAPNLSYDTIISEDLHAPDGLAVDWIHRNIYWTDSVPGSVSVADTKGVKRRTLFQEAGSRPRAIVVDPVHGFMYWTDWGTPAKIKKGGLNGVDIHSLVTENIQWPNGITLDLSSGRLYWVDSKLHSISSIDVNGGNRKTILEDENRLAHPFSLAIYEDKVYWTDVINEAIFSANRLTGSDVNLVAENLLSPEDIVLFHKVTQPRGVNWCETTALLPNGGCQYLCLPAPQIGPHSPKFTCACPDGMLLAKDMRSCLTEVDTVLTTQGTSAVRPVVTASATRPPKHSEDLSAPSTPRQPVDTPGLSTVASVTVSHQVQGDMAGRGNEEQPHGMRFLSIFFPIALVALLVLGAVLLWRNWRLKNINSINFDNPVYQKTTEDELHICRSQDGYTYPSRQMVSLEDDVA

SEQ ID NO: 4 - protein, 2215 aa, Mus musculus
MATRSSRRESRLPFLFALVALLPRGALGGGWTQRLHGGPAPLPQDRGFFVVQGDPRDLRLGTHGDAPGASPAARKPLRTRRSAALQPQPIQVYGQVSLNDSHNQMVVHWAGEKSNVIVALARDSLALARPKSSDVYVSYDYGKSFSKISEKLNFGVGNNSEAVISQFYHSPADNKRYIFVDAYAQYLWITFDFCSTIHGFSIPFRAADLLLHSKASNLLLGFDRSHPNKQLWKSDDFGQTWIMIQEHVKSFSWGIDPYDQPNAIYIERHEPFGFSTVLRSTDFFQSRENQEVILEEVRDFQLRDKYMFATKVVHLPGSQQQSSVQLWVSFGRKPMRAAQFVTKHPINEYYIADAAEDQVFVCVSHSNNSTNLYISEAEGLKFSLSLENVLYYSPGGAGSDTLVRYFANEPFADFHRVEGLQGVYIATLINGSMNEENMRSVITFDKGGTWEFLQAPAFTGYGEKINCELSQGCSLHLAQRLSQLLNLQLRRMPILSKESAPGLIIATGSVGKNLASKTNVYISSSAGARWREALPGPHYYTWGDHGGIIMAIAQGMETNELKYSTNEGETWKTFVFSEKPVFVYGLLTEPGEKSTVFTIFGSNKESVHSWLILQVNATDALGVPCTENDYKLWSPSDERGNECLLGHKTVFKRRTPHATCFNGEDFDRPVVVSNCSCTREDYECDFGFKMSEDLSLEVCVPDPEFSGKPYSPPVPCPVGSSYRRTRGYRKISGDTCSGGDVEARLEGELVPCPLAEENEFILYAMRKSIYRYDLASGATEQLPLWGLRAAVALDFDYERNCLYWSDLALDTIQRLCLNGSTGQEVIINSGLETVEALAFEPLSQLLYWVDAGFKKIEVANPDGDFRLTIVNSSVLDRPRALVLVPQEGVMFWTDWGDLKPGIYRSYMDGSAAYRLVSEDVKWPNGISVDSQWIYWTDAYLDCIERITFSGQQGSVILDSLPHPYAIAVFKNEIYWDDWSQLSIFRASKHSRSQVEILASQLTGLMDMKVFYKGKNAGSNACVPQPCSLLCLPKANNSKSCRCPEGVASSVLPSGDLMCDCPQGYQRKNNTCVKEENTCLRNQYRCSNGNCINSIWWCDFDNDCGDMSDERNCPTTVCDADTQFRCQESGTCIPLSYKCDLEDDCGDNSDESHCEMHQCRSDEFNCSSGMCIRSSWVCDGDNDCRDWSDEANCTAIYHTCEASNFQCHNGHCIPQRWACDGDADCQDGSDEDPVSCEKKCNGFHCPNGTCIPSSKHCDGLRDCPDGSDEQHCEPFCTRFMDFVCKNRQQCLFHSMVCDGIVQCRDGSDEDAAFAGCSQDPEFHKECDEFGFQCQNGVCISLIWKCDGMDDCGDYSDEANCENPTEAPNCSRYFQFHCENGHCIPNRWKCDRENDCGDWSDEKDCGDSHVLPSPTPGPSTCLPNYFRCSSGACVMGTWVCDGYRDCADGSDEEACPSLANSTAASTPTQLGQCDRFEFECHQPKKCIPNWKRCDGHQDCQDGQDEANCPTHSTLTCTSREFKCEDGEACIVLSERCDGFLDCSDESDEKACSDELTVYKVQNLQWTADFSGDVTLTWMRPKKMPSASCVYNVYYRVVGESIWKTLETHSNKTSTVLKVLKPDTTYQVKVQVHCLNKVHNTNDFVTLRTPEGLPDAPRNLQLSLNREEEGVILGHWAPPVHTHGLIREYIVEYSRSGSKMWASQRAASNSTEIKNLLLNALYTVRVAAVTSRGIGNWSDSKSITTIKGKVIQAPNIHIDSYDENSLSFTLTMDGDIKVNGYVVNLFWSFDAHKQEKKTLSFRGGSALSHKVSNLTAHTSYEISAWAKTDLGDSPLAFEHILTRGSSPPAPSLKAKAINQTAVECIWTGPKNVVYGIFYATSFLDLYRNPKSVTTSLHNKTVIVSKDEQYLFLVRVLIPYQGPSSDYVVVKMIPDSRLPPRHLHAVHIGKTSALIKWESPYDSPDQDLFYAIAVKDLIRKTDRSYKVRSRNSTVEYSLSKLEPGGKYHIIVQLGNMSKDSSIKITTVSLSAPDALKIITENDHVLLFWKSLALKEKQFNETRGYEIHMSDSAVNLTAYLGNTTDNFFKVSNLKMGHNYTFTVQARCLFGSQICGEPAVLLYDELSSGADAAVIHAARSTDVAAVVVPILFLILLSLGVGFAILYTKHRRLQSSFSAFANSHYSSRLGSAIFSSGDDLGEDDEDAPMITGFSDDVPMVIA

SEQ ID NO: 5 - protein, 888 aa, Mus musculus
MGRVPLAWWLALCCWGCAAHKDTQTEAGSPFVGNPGNITGARGLTGTLRCELQVQGEPPEVVWLRDGQILELADNTQTQVPLGEDWQDEWKVVSQLRISALQLSDAGEYQCMVHLEGRTFVSQPGFVGLEGLPYFLEEPEDKAVPANTPFNLSCQAQGPPEPVTLLWLQDAVPLAPVTGHSSQHSLQTPGLNKTSSFSCEAHNAKGVTTSRTATITVLPQRPHHLHVVSRQPTELEVAWTPGLSGIYPLTHCNLQAVLSDDGVGIWLGKSDPPEDPLTLQVSVPPHQLRLEKLLPHTPYHIRISCSSSQGPSPWTHWLPVETTEGVPLGPPENVSAMRNGSQVLVRWQEPRVPLQGTLLGYRLAYRGQDTPEVLMDIGLTREVTLELRGDRPVRNLTVSVTAYTSAGDGPWSLPVPLEPWRPGQGQPLHHLVSEPPPRAFSWPWWYVLLGALVAAACVLILALFLVHRRKKETRYGEVFEPTVERPELVVRYRVRKSYSRRTTEATLNSLGISEELKEKLRDVMVDRHKVALGKTLGEGEFGAVMEGQLNQDDSILKVAVKTMKIVICTRSELEDFLSEAVCMKEFDHPNVMRLIGVCFQGSDREGFPEPVVILPFMKHGDLHSFLLYSRLGDQPVFLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPVDCLDGLYALMSRCWELNPRDRPSFAELREDLENTLKALPPAQEPDEILYVNMDEGGSHLEPRGAAGGADPPTQPDPKDSCSCLTAADVHSAGRYVLCPSTAPGPTLSADRGCPAPPGQEDGA

SEQ ID NO: 6 - protein, 513 aa, Mus musculus
MEPAGPAPGRLGPLLLCLLLSASCFCTGVTGKELKVTQPEKSVSVAAGDSTVLNCTLTSLLPVGPIKWYRGVGQSRLLIYSFTGEHFPRVTNVSDATKRNNMDFSIRISNVTPEDAGTYYCVKFQKGPSEPDTEIQSGGGTEVYVLAKPSPPEVSGPADRGIPDQKVNFTCKSHGFSPRNITLKWFKDGQELHHLETTVNPSGKNVSYNISSTVRVVLNSMDVHSKVICEVAHITLDRSPLRGIANLSNFIRVSPTVKVTQQSPTSMNQVNLTCRAERFYPEDLQLIWLENGNVSRNDTPKNLTKNTDGTYNYTSLFLVNSSAHREDVVFTCQVKHDQQPAITRNHTVLGLAHSSDQGSMQTFPGNNATHNWNVFIGVGVACALLVVLLMAALYLLRIKQKKAKGSTSSTRLHEPEKNAREITQVQSLIQDTNDINDITYADLNLPKEKKPAPRAPEPNNHTEYASIETGKVPRPEDTLTYADLDMVHLSRAQPAPKPEPSFSEYASVQVQRK

SEQ ID NO: 7 - protein, 1218 aa, Mus musculus
MRSPRTRGRPGRPLSLLLALLCALRAKVCGASGQFELEILSMQNVNGELQNGNCCGGVRNPGDRKCTRDECDTYFKVCLKEYQSRVTAGGPCSFGSGSTPVIGGNTFNLKASRGNDRNRIVLPFSFAWPRSYTLLVEAWDSSNDTIQPDSIIEKASHSGMINPSRQWQTLKQNTGIAHFEYQIRVTCDDHYYGFGCNKFCRPRDDFFGHYACDQNGNKTCMEGWMGPDCNKAICRQGCSPKHGSCKLPGDCRCQYGWQGLYCDKCIPHPGCVHGTCNEPWQCLCETNWGGQLCDKDLNYCGTHQPCLNRGTCSNTGPDKYQCSCPEGYSGPNCEIAEHACLSDPCHNRGSCKETSSGFECECSPGWTGPTCSTNIDDCSPNNCSHGGTCQDLVNGFKCVCPPQWTGKTCQLDANECEAKPCVNARSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTTCEVIDSCTVAMASNDTPEGVRYISSNVCGPHGKCKSQSGGKFTCDCNKGFTGTYCHENINDCESNPCKNGGTCIDGVNSYKCICSDGWEGAHCENNINDCSQNPCHYGGTCRDLVNDFYCDCKNGWKGKTCHSRDSQCDEATCNNGGTCYDEVDTFKCMCPGGWEGTTCNIARNSSCLPNPCHNGGTCVVNGDSFTCVCKEGWEGPICTQNTNDCSPHPCYNSGTCVDGDNWYRCECAPGFAGPDCRININECQSSPCAFGATCVDEINGYQCICPPGHSGAKCHEVSGRSCITMGRVILDGAKWDDDCNTCQCLNGRVACSKVWCGPRPCRLHKSHNECPSGQSCIPVLDDQCFVRPCTGVGECRSSSLQPVKTKCTSDSYYQDNCANITFTFNKEMMSPGLTTEHICSELRNLNILKNVSAEYSIYIACEPSLSANNEIHVAISAEDIRDDGNPVKEITDKIIDLVSKRDGNSSLIAAVAEVRVQRRPLKNRTDFLVPLLSSVLTVAWVCCLVTAFYWCVRKRRKPSSHTHSAPEDNTTNNVREQLNQIKNPIEKHGANTVPIKDYENKNSKMSKIRTHNSEVEEDDMDKHQQKVRFAKQPVYTLVDREEKAPSGTPTKHPNWTNKQDNRDLESAQSLNRMEYIV

SEQ ID NO: 8 - protein, 322 aa, Mus musculus
MQLKCPCFVSLGTRQPVWKKLHVSSGFFSGLGLFLLLLSSLCAASAETEVGAMVGSNVVLSCIDPHRRHFNLSGLYVYWQIENPEVSVTYYLPYKSPGINVDSSYKNRGHLSLDSMKQGNFSLYLKNVTPQDTQEFTCRVFMNTATELVKILEEVVRLRVAANFSTPVISTSDSSNPGQERTYTCMSKNGYPEPNLYWINTTDNSLIDTALQNNTVYLNKLGLYDVISTLRLPWTSRGDVLCCVENVALHQNITSISQAESFTGNNTKNPQETHNNELKVLVPVLAVLAAAAFVSFIIYRRTRPHRSYTGPKTVQLELTDHA

SEQ ID NO: 9 - protein, 347 aa, Mus musculus
MQLKCPCFVSLGTRQPVWKKLHVSSGFFSGLGLFLLLLSSLCAASAETEVGAMVGSNVVLSCIDPHRRHFNLSGLYVYWQIENPEVSVTYYLPYKSPGINVDSSYKNRGHLSLDSMKQGNFSLYLKNVTPQDTQEFTCRVFMNTATELVKILEEVVRLRVAANFSTPVISTSDSSNPGQERTYTCMSKNGYPEPNLYWINTTDNSLIDTALQNNTVYLNKLGLYDVISTLRLPWTSHGDVLCCVENVALHQNITSISQAESFTGNNTKNPQETHNNELKVLVPVLAVLAAAAFVSFIIYRRTRPHRSYTGPKTVQLELTDTWAPVPYQDYLIPRYLMSPCLKTRGLP

SEQ ID NO: 10 - protein, 366 aa, Mus musculus
MERVLGLLLLLLVHASPAPPEPCELDEESCSCNFSDPKPDWSSAFNCLGAADVELYGGGRSLEYLLKRVDTEADLGQFTDIIKSLSLKRLTVRAARIPSRILFGALRVLGISGLQELTLENLEVTGTAPPPLLEATGPDLNILNLRNVSWATRDAWLAELQQWLKPGLKVLSIAQAHSLNFSCEQVRVFPALSTLDLSDNPELGERGLISALCPLKFPTLQVLALRNAGMETPSGVCSALAAARVQLQGLDLSHNSLRDAAGAPSCDWPSQLNSLNLSFTGLKQVPKGLPAKLSVLDLSYNRLDRNPSPDELPQVGNLSLKGNPFLDSESHSEKFNSGVVTAGAPSSQAVALSGTLALLLGDRLFV

SEQ ID NO: 11 - protein, 770 aa, Mus musculus
MLGPHSLLLALAGLFFLGSAVSQECTKYKVSSCRDCIQSGPGCSWCQKLNFTGPGEPDSLRCDTRAQLLLKGCPADDIMDPRSIANPEFDQRGQRKQLSPQKVTLYLRPGQAAAFNVTFRRAKGYPIDLYYLMDLSYSMLDDLNNVKKLGGDLLQALNEITESGRIGFGSFVDKTVLPFVNTHPEKLRNPCPNKEKACQPPFAFRHVLKLTDNSNQFQTEVGKQLISGNLDAPEGGLDAIMQVAACPEEIGWRNVTRLLVFATDDGFHFAGDGKLGAILTPNDGRCHLEDNMYKRSNEFDYPSVGQLAHKLSESNIQPIFAVTKKMVKTYEKLTEIIPKSAVGELSDDSSNVVQLIKNAYYKLSSRVFLDHSTLPDTLKVTYDSFCSNGASSIGKSRGDCDGVQINNPVTFQVKVMASECIQEQSFVIRALGFTDTVTVQVRPQCECQCRDQSREQSLCGGKGVMECGICRCESGYIGKNCECQTQGRSSQELERNCRKDNSSIVCSGLGDCICGQCVCHTSDVPNKEIFGQYCECDNVNCERYNSQVCGGSDRGSCNCGKCSCKPGYEGSACQCQRSTTGCLNARLVECSGRGHCQCNRCICDEGYQPPMCEDCPSCGSHCRDNHTSCAECLKFDKGPFEKNCSVQCAGMTLQTIPLKKKPCKERDSEGCWITYTLQQKDGRNIYNIHVEDSLECVKGPNVAAIVGGTVVGVVLIGVLLLVIWKALTHLTDLREYRRFEKEKLKSQWNNDNPLFKSATTTVMNPKFAES

SEQ ID NO: 12 - protein, 530 aa, Mus musculus
MARFRRADLAAAGVMLLCHFLTDRFHFAHGEPGHHTNDWIYEVTNAFPWNEEGVEVDSQAYNHRWKRNVDPFKAVDTNRASMGQASPESKGFTDLLLDDGQDNNTQIEEDTDHNYYISRIYGPADSASRDLWVNIDQMEKDKVKIHGILSNTHRQAARVNLSFDFPFYGHFLNEVTVATGGFIYTGEVVHRMLTATQYIAPLMANFDPSVSRNSTVRYFDNGTALVVQWDHVHLQDNYNLGSFTFQATLLMDGRIIFGYKEIPVLVTQISSTNHPVKVGLSDAFVVVHRIQQIPNVRRRTIYEYHRVELQMSKITNISAVEMTPLPTCLQFNGCGPCVSSQIGFNCSWCSKLQRCSSGFDRHRQDWVDSGCPEEVQSKEKMCEKTEPGETSQTTTTSHTTTMQFRVLTTTRRAVTSQMPTSLPTEDDTKIALHLKDSGASTDDSAAEKKGGTLHAGLIVGILILVLIIAAAILVTVYMYHHPTSAASIFFIERRPSRWPAMKFRRGSGHPAYAEVEPVGEKEGFIVSEQC

SEQ ID NO: 13 - protein, 16 aa, Artificial Sequence
  peptide
CLVGEFVSDALLVPDK

SEQ ID NO: 14 - protein, 15 aa, Artificial Sequence
  peptide
MDVCETHLHWHTVAK

SEQ ID NO: 15 - protein, 13 aa, Artificial Sequence
  peptide
CVPFFYGGCGGNR

SEQ ID NO: 16 - protein, 17 aa, Artificial Sequence
  peptide
STNLHDYGMLLPCGIDK

SEQ ID NO: 17 - protein, 17 aa, Artificial Sequence
  peptide
EQNYSDDVLANMISEPR

SEQ ID NO: 18 - protein, 12 aa, Artificial Sequence
  peptide
VESLEQEAANER

SEQ ID NO: 19 - protein, 16 aa, Artificial Sequence
  peptide
ISYGNDALMPSLTETK

SEQ ID NO: 20 - protein, 9 aa, Artificial Sequence
  peptide
WYFDVTEGK

SEQ ID NO: 21 - protein, 14 aa, Artificial Sequence
  peptide
MDAEFGHDSGFEVR

SEQ ID NO: 22 - protein, 16 aa, Artificial Sequence
  peptide
YLETPGDENEHAHFQK

SEQ ID NO: 23 - protein, 16 aa, Artificial Sequence
  peptide
CELQVQGEPPEVVWLR

SEQ ID NO: 24 - protein, 21 aa, Artificial Sequence
  peptide
DTQTEAGSPFVGNPGDITGAR

SEQ ID NO: 25 - protein, 11 aa, Artificial Sequence
  peptide
VPLQGTLLGYR

SEQ ID NO: 26 - protein, 21 aa, Artificial Sequence
  peptide
ISALQLSDAGEYQCMVHLEGR

SEQ ID NO: 27 - protein, 15 aa, Artificial Sequence
  peptide
GQDTPEVLMDIGLTR

SEQ ID NO: 28 - protein, 7 aa, Artificial Sequence
  peptide
EVTLELR

SEQ ID NO: 29 - protein, 8 aa, Artificial Sequence
  peptide
EDLHTDFK

SEQ ID NO: 30 - protein, 24 aa, Artificial Sequence
  peptide
GTTTEPIPVIISPLETIPASLGSR

SEQ ID NO: 31 - protein, 10 aa, Artificial Sequence
  peptide
EFLSAGDPTR

SEQ ID NO: 32 - protein, 22 aa, Artificial Sequence
  peptide
GNILWILPAVQQDSGTYICTFR

SEQ ID NO: 33 - protein, 9 aa, Artificial Sequence
  peptide
LDSSQLIPR

SEQ ID NO: 34 - protein, 9 aa, Artificial Sequence
  peptide
LEGEPVVLR

SEQ ID NO: 35 - protein, 13 aa, Artificial Sequence
  peptide
GNKEFLSAGDPTR

SEQ ID NO: 36 - protein, 26 aa, Artificial Sequence
  peptide
VKGTTTEPIPVIISPLETIPASLGSR

SEQ ID NO: 37 - protein, 13 aa, Artificial Sequence
  peptide]
DASHCEQMSVELK

SEQ ID NO: 38 - protein, 15 aa, Artificial Sequence
  peptide
LLISDTSMDDAGYYR

SEQ ID NO: 39 - protein, 12 aa, Artificial Sequence
  peptide
EWTTTGNTLVLR

SEQ ID NO: 40 - protein, 14 aa, Artificial Sequence
  peptide
SDFQVPCQYSQQLK

SEQ ID NO: 41 - protein, 24 aa, Artificial Sequence
  peptide
ALEVADGTVTSLPGATVTLICPGK

SEQ ID NO: 42 - protein, 13 aa, Artificial Sequence
  peptide
AVGSIGYLLFTNR

SEQ ID NO: 43 - protein, 7 aa, Artificial Sequence
  peptide
LYWVDSK

SEQ ID NO: 44 - protein, 11 aa, Artificial Sequence
  peptide
CHSGECISLDK

SEQ ID NO: 45 - protein, 18 aa, Artificial Sequence
  peptide
NIYWTDSVPGSVSVADTK

SEQ ID NO: 46 - protein, 12 aa, Artificial Sequence
  peptide
IGSECLCPSGFR

SEQ ID NO: 47 - protein, 13 aa, Artificial Sequence
  peptide
NVVALDTEVTNNR

SEQ ID NO: 48 - protein, 9 aa, Artificial Sequence
  peptide
IYWSDLSQK

SEQ ID NO: 49 - protein, 11 aa, Artificial Sequence
  peptide
SEYTSLLPNLK

SEQ ID NO: 50 - protein, 13 aa, Artificial Sequence
  peptide
LAHPFSLAIYEDK

SEQ ID NO: 51 - protein, 11 aa, Artificial Sequence
  peptide
SWVCDGEADCK

SEQ ID NO: 52 - protein, 8 aa, Artificial Sequence
  peptide
TILEDENR

SEQ ID NO: 53 - protein, 24 aa, Artificial Sequence
  peptide
LTGSDVNLVAENLLSPEDIVLFHK

SEQ ID NO: 54 - protein, 14 aa, Artificial Sequence
  peptide
LHSISSIDVDGGNR

SEQ ID NO: 55 - protein, 11 aa, Artificial Sequence
  peptide
EIEYLENTLPK

SEQ ID NO: 56 - protein, 11 aa, Artificial Sequence
  peptide
SKEDCVEIYIK

SEQ ID NO: 57 - protein, 12 aa, Artificial Sequence
  peptide
QNYTDLVAIQNK

SEQ ID NO: 58 - protein, 10 aa, Artificial Sequence
  peptide
SPYYYWIGIR

SEQ ID NO: 59 - protein, 9 aa, Artificial Sequence
  peptide
MWTWVGTDK

SEQ ID NO: 60 - protein, 12 aa, Artificial Sequence
  peptide
QDYTDLVAIQNK

SEQ ID NO: 61 - protein, 17 aa, Artificial Sequence
  peptide
AALCYTASCQPGSCDGR

SEQ ID NO: 62 - protein, 14 aa, Artificial Sequence
  peptide
ASEAGQYFLMAQNK

SEQ ID NO: 63 - protein, 13 aa, Artificial Sequence
  peptide
VIIQSQLPIGTLK

SEQ ID NO: 64 - protein, 8 aa, Artificial Sequence
  peptide
KLEFITQR

SEQ ID NO: 65 - protein, 10 aa, Artificial Sequence
  peptide
VLDSNTYVCK

SEQ ID NO: 66 - protein, 9 aa, Artificial Sequence
  peptide
TVYFFSPWR

SEQ ID NO: 67 - protein, 14 aa, Artificial Sequence
  peptide
TGPVLEHPDCLPCR

SEQ ID NO: 68 - protein, 18 aa, Artificial Sequence
  peptide
YIHAFESNHFIYFLTVQK

SEQ ID NO: 69 - protein, 11 aa, Artificial Sequence
  peptide
ETLDAQTFHTR

SEQ ID NO: 70 - protein, 25 aa, Artificial Sequence
  peptide
DNINMALLVDTYYDDQLISCGSVNR

SEQ ID NO: 71 - protein, 23 aa, Artificial Sequence
  peptide
FCSVDSGLHSYMEMPLECILTEK

SEQ ID NO: 72 - protein, 26 aa, Artificial Sequence
  peptide
FINFFVGNTIDSSYPPGYSLHSISVR

SEQ ID NO: 73 - protein, 13 aa, Artificial Sequence
  peptide
LLIYSFTGEHFPR

SEQ ID NO: 74 - protein, 15 aa, Artificial Sequence
  peptide
EVVDSYLPVILDMIK

SEQ ID NO: 75 - protein, 14 aa, Artificial Sequence
  peptide
QLESNKIPEVDMAR

SEQ ID NO: 76 - protein, 10 aa, Artificial Sequence
  peptide
LVSDVQTAVK

SEQ ID NO: 77 - protein, 20 aa, Artificial Sequence
  peptide
VVAPFMSNIPLLLYPQDHPR

SEQ ID NO: 78 - protein, 14 aa, Artificial Sequence
  peptide
TDSSFIQGFVDHVK

SEQ ID NO: 79 - protein, 9 aa, Artificial Sequence
  peptide
YLTVAAVFR

SEQ ID NO: 80 - protein, 12 aa, Artificial Sequence
  peptide
IMNTFSVVPSPK

SEQ ID NO: 81 - protein, 14 aa, Artificial Sequence
  peptide
LHFFMPGFAPLTSR

SEQ ID NO: 82 - protein, 15 aa, Artificial Sequence
  peptide
ALTVPELTQQVFDAK

SEQ ID NO: 83 - protein, 18 aa, Artificial Sequence
  peptide
GHYTEGAELVDSVLDVVR

SEQ ID NO: 84 - protein, 14 aa, Artificial Sequence
  peptide
LFGFCPLHFEGSER

SEQ ID NO: 85 - protein, 12 aa, Artificial Sequence
  peptide
IGSECLCPSGFR

SEQ ID NO: 86 - protein, 13 aa, Artificial Sequence
  peptide
FTCACPDGMLLAK

SEQ ID NO: 87 - protein, 13 aa, Artificial Sequence
  peptide
EVCSEQAETGPCR

SEQ ID NO: 88 - protein, 13 aa, Artificial Sequence
  peptide
CVPFFYGGCGGNR

SEQ ID NO: 89 - protein, 11 aa, Artificial Sequence
  peptide
TCSGGSAVLCR

SEQ ID NO: 90 - protein, 8 aa, Artificial Sequence
  peptide
VCNPIITK

SEQ ID NO: 91 - protein, 10 aa, Artificial Sequence
  peptide
CNEIISWLDK

SEQ ID NO: 92 - protein, 9 aa, Artificial Sequence
  peptide
IGVCPSAYK

SEQ ID NO: 93 - protein, 14 aa, Artificial Sequence
  peptide
GLSNACALLPDPAR

SEQ ID NO: 94 - protein, 12 aa, Artificial Sequence
  peptide
TSSFSCEAHNAK

SEQ ID NO: 95 - protein, 16 aa, Artificial Sequence
  peptide
CELQVQGEPPEVVWLR

SEQ ID NO: 96 - protein, 21 aa, Artificial Sequence
  peptide
ISALQLSDAGEYQCMVHLEGR

SEQ ID NO: 97 - protein, 11 aa, Artificial Sequence
  peptide
TPFYVCPGEGR

SEQ ID NO: 98 - protein, 10 aa, Artificial Sequence
  peptide
EDVVFTCQVK

SEQ ID NO: 99 - protein, 12 aa, Artificial Sequence
  peptide
VICEVAHITLDR

SEQ ID NO: 100 - protein, 10 aa, Artificial Sequence
  peptide
GLISALCPLK

SEQ ID NO: 101 - protein, 12 aa, Artificial Sequence
  peptide
AGWCPGMAVPTR

SEQ ID NO: 102 - protein, 7 aa, Artificial Sequence
  peptide
FMDFVCK

SEQ ID NO: 103 - protein, 10 aa, Artificial Sequence
  peptide
EDYECDFGFK

SEQ ID NO: 104 - protein, 16 aa, Artificial Sequence
  peptide
INCELSQGCSLHLAQR

SEQ ID NO: 105 - protein, 16 aa, Artificial Sequence
  peptide
EFKCEDGEACIVLSER

SEQ ID NO: 106 - protein, 17 aa, Artificial Sequence
  peptide
DCPDGSDEQHCEPFCTR

SEQ ID NO: 107 - protein, 14 aa, Artificial Sequence
  peptide
CKPGFFNLESSNPK

SEQ ID NO: 108 - protein, 10 aa, Artificial Sequence
  peptide
TCHIQECDKR

SEQ ID NO: 109 - protein, 14 aa, Artificial Sequence
  peptide
NTDPGYNCLPCPPR

SEQ ID NO: 110 - protein, 11 aa, Artificial Sequence
  peptide
SVFACQEQVLK

SEQ ID NO: 111 - protein, 13 aa, Artificial Sequence
  peptide
CVPFFYGGCGGNR

SEQ ID NO: 112 - protein, 17 aa, Artificial Sequence
  peptide
STNLHDYGMLLPCGIDK

SEQ ID NO: 113 - protein, 7 aa, Artificial Sequence
  peptide
CIPDSWR

SEQ ID NO: 114 - protein, 13 aa, Artificial Sequence
  peptide
FTCACPDGMLLAK

SEQ ID NO: 115 - protein, 13 aa, Artificial Sequence
  peptide
FTCACPDGMLLAK

SEQ ID NO: 116 - protein, 14 aa, Artificial Sequence
  peptide
DCQDWSDEPIKECK

SEQ ID NO: 117 - protein, 13 aa, Artificial Sequence
  peptide
CVPFFYGGCGGNR

SEQ ID NO: 118 - protein, 17 aa, Artificial Sequence
  peptide
STNLHDYGMLLPCGIDK

SEQ ID NO: 119 - protein, 7 aa, Artificial Sequence
  peptide
CVASNPR

SEQ ID NO: 120 - protein, 13 aa, Artificial Sequence
  peptide
DASHCEQMSVELK

SEQ ID NO: 121 - protein, 22 aa, Artificial Sequence
  peptide
GNILWILPAVQQDSGTYICTFR

SEQ ID NO: 122 - protein, 21 aa, Artificial Sequence
  peptide
CPLAPHSDISSSSHSFLTWSK

SEQ ID NO: 123 - protein, 29 aa, Artificial Sequence
  peptide
NTEASLPHVSYLQISALSTTGLLVCPDLK

SEQ ID NO: 124 - protein, 14 aa, Artificial Sequence
  peptide
SDFQVPCQYSQQLK

SEQ ID NO: 125 - protein, 24 aa, Artificial Sequence
  peptide
ALEVADGTVTSLPGATVTLICPGK

SEQ ID NO: 126 - protein, 11 aa, Artificial Sequence
  peptide
GCSFLPDPYQK

SEQ ID NO: 127 - protein, 11 aa, Artificial Sequence
  peptide
TCSGGSAVLCR

SEQ ID NO: 128 - protein, 13 aa, Artificial Sequence
  peptide
EICVLAGFCNEVK

SEQ ID NO: 129 - protein, 17 aa, Artificial Sequence
  peptide
CVWGPSYWCQNMETAAR

SEQ ID NO: 130 - protein, 8 aa, Artificial Sequence
  peptide
SCNCLLLK

SEQ ID NO: 131 - protein, 15 aa, Artificial Sequence
  peptide
VNQIGSVTESLQACK

SEQ ID NO: 132 - protein, 16 aa, Artificial Sequence
  peptide
CELQVQGEPPEVVWLR

SEQ ID NO: 133 - protein, 8 aa, Artificial Sequence
  peptide
CPEDYEGK

SEQ ID NO: 134 - protein, 8 aa, Artificial Sequence
  peptide
DECDTYFK

SEQ ID NO: 135 - protein, 13 aa, Artificial Sequence
  peptide
CECAPGFAGPDCR

SEQ ID NO: 136 - protein, 13 aa, Artificial Sequence
  peptide
CFLGCELPPEGSR

SEQ ID NO: 137 - protein, 12 aa, Artificial Sequence
  peptide
VICEVAHITLDR

SEQ ID NO: 138 - protein, 8 aa, Artificial Sequence
  peptide
NNMDFSIR

SEQ ID NO: 139 - protein, 11 aa, Artificial Sequence
  peptide
VVLNSMDVHSK

SEQ ID NO: 140 - protein, 13 aa, Artificial Sequence
  peptide
LLIYSFTGEHFPR

SEQ ID NO: 141 - protein, 10 aa, Artificial Sequence
  peptide
VLDSNTYVCK

SEQ ID NO: 142 - protein, 8 aa, Artificial Sequence
  peptide
KLEFITQR

SEQ ID NO: 143 - protein, 13 aa, Artificial Sequence
  peptide
VIIQSQLPIGTLK

SEQ ID NO: 144 - protein, 14 aa, Artificial Sequence
  peptide
ASEAGQYFLMAQNK

SEQ ID NO: 145 - protein, 8 aa, Artificial Sequence
  peptide
SQVMTHLR

SEQ ID NO: 146 - protein, 10 aa, Artificial Sequence
  peptide
QQLVETHMAR

SEQ ID NO: 147 - protein, 12 aa, Artificial Sequence
  peptide
TSSFSCEAHNAK

SEQ ID NO: 148 - protein, 18 aa, Artificial Sequence
  peptide
NAGMETPSGVCSALAAAR

SEQ ID NO: 149 - protein, 16 aa, Artificial Sequence
  peptide
GQGCPDYVLLTHTVSR

SEQ ID NO: 150 - protein, 8 aa, Artificial Sequence
  peptide
VCACEAGR

SEQ ID NO: 151 - protein, 13 aa, Artificial Sequence
  peptide
NVTPQDTQEFTCR

SEQ ID NO: 152 - protein, 7 aa, Artificial Sequence
  peptide
TYTCMSK

SEQ ID NO: 153 - protein, 11 aa, Artificial Sequence
  peptide
LGLYDVISTLR

SEQ ID NO: 154 - protein, 11 aa, Artificial Sequence
  peptide
VFMNTATELVK

SEQ ID NO: 155 - protein, 9 aa, Artificial Sequence
  peptide
STTGCLNAR

SEQ ID NO: 156 - protein, 11 aa, Artificial Sequence
  peptide
YNSQVCGGSDR

SEQ ID NO: 157 - protein, 19 aa, Artificial Sequence
  peptide
SRGDCDGVQINNPVTFQVK

SEQ ID NO: 158 - protein, 17 aa, Artificial Sequence
  peptide
HRQDWVDSGCPEEVQSK

SEQ ID NO: 159 - protein, 860 aa, Homo sapiens [sequence omitted]

(19) United States
(12) Patent Application Publication
(10) Pub. No.: US 2009/0247605 A1
(43) Pub. Date: Oct. 1, 2009

(54) Treating diseases mediated by metalloprotease-shed proteins

(75) Inventors: Richard S. Johnson, Mercer Island, WA (US); Lin Guo, Seattle, WA (US); Jacques J. Peschon, Seattle, WA (US); Roy A. Black, Seattle, WA (US); Rajeev M. Mahimkar, San Francisco, CA (US)

Correspondence Address: IMMUNEX CORPORATION;LAW DEPARTMENT, 1201 AMGEN COURT WEST, SEATTLE, WA, 98119, US

(73) Assignee: Immunex Corporation, Thousand Oaks, CA (US)

(21) Appl. No.: 11/824,227

(22) Filed: Jun. 28, 2007

Related U.S. Application Data
(63) Continuation of application No. 10/281,478, filed on Oct. 25, 2002, now abandoned.
(60) Provisional application No. 60/353,387, filed on Oct. 26, 2001.

(51) Int. Cl. A61K 31/7088; C12Q 1/37; G01N 33/53

(52) U.S. Cl. 514/44 A; 435/23; 435/7.1

(57) ABSTRACT

This invention relates to the identification of membrane-associated proteins shed by metalloproteinases and in particular by TNF-alpha converting enzyme (TACE), to the use of such metalloproteinase-shed proteins in assays for TACE agonists and antagonists, and to the use of metalloproteinase agonists and antagonists, and particularly TACE agonists and antagonists, in the treatment of diseases mediated by certain shed proteins.

This application is a continuation of U.S. patent application Ser. No. 10/281,478, filed Oct. 25, 2002, which claims the benefit under 35 U.S.C. 119(e) of U.S. provisional application Ser. No. 60/353,387, filed Oct. 26, 2001, which is incorporated in its entirety by reference herein.

### FIELD OF THE INVENTION

This invention relates to the identification of membrane-associated proteins shed by metalloproteinases and in particular by TNF-alpha converting enzyme (TACE), to the use of such metalloproteinase-shed proteins in assays for inhibitors of TACE, and to the use of agonists and antagonists of metalloproteinases, and of TACE in particular, in the treatment of diseases mediated by certain shed proteins.

### BACKGROUND OF THE INVENTION

Proteolytic cleavage (shedding) of extracellular domains of many membrane proteins by metalloproteases is an important regulatory mechanism used by mammalian cells in response to environmental and physiological changes. Proteolysis of cell membrane-bound proteins provides a post-translational means of regulating protein function, and has been shown to control the production of many soluble cytokines, receptors, adhesion molecules and growth factors through the process termed “ectodomain shedding” (Schlondorff and Blobel, 1999, J Cell Sci 112: 3603-3617; Mullberg et al., 2000, Eur Cytokine Netw 11: 27-38). Abnormal shedding can contribute to diseases such as rheumatoid arthritis and cancer (Blobel, 2000, Curr Opin Cell Biol 12: 606-612). A key player in ectodomain shedding is the ADAM (A disintegrin and metalloprotease) family of metalloproteases. ADAMs are characterized by a conserved domain structure that consists of an N-terminal signal sequence followed by the pro-domain, the metalloprotease and disintegrin domains, a cysteine-rich region usually containing an EGF repeat, a transmembrane domain, and a cytoplasmic tail (Black and White, 1998, Curr Opin Cell Biol 10: 654-659).

Tumor necrosis factor-alpha converting enzyme (TACE, also called ADAM-17) was the first ADAM family protease to be characterized as a “sheddase”. It was originally identified by its ability to cleave membrane-bound proTNF-alpha, resulting in the release of soluble TNF-alpha from cells (Black et al., 1997, Nature 385: 729-733; Moss et al., 1997, Nature 385: 733-736). Subsequent work, primarily involving TACE knockout mice and cells, indicated that the shedding of a number of other proteins is mediated by TACE. These include transforming growth factor-alpha (TGF-alpha), L-selectin, p75 TNFR, amyloid A4 protein, CD30, IL-6 receptor type 1 (IL-6R-1), Notch1 receptor, growth hormone binding protein, and macrophage colony-stimulating factor receptor (M-CSFR) (Peschon et al., 1998, Science 282: 1281-1284; Buxbaum et al., 1998 J Biol Chem 273: 27765-27767; Brou et al., 2000, Molecular Cell 5: 207-216; Hansen et al., 2000, J Immunol 165: 6703-6709; Zhang et al., 2000, Endocrinology 141: 4342-4348; Rovida et al., 2001, J Immunol 166: 1583-1589; and Althoff et al., 2000, Eur J Biochem 267: 2624-2631). In all of these studies, the discovery that the protein was shed by TACE was made through a hypothesis-driven approach, rather than an unbiased screening process.

Identification of membrane-associated proteins previously not known to be shed by TACE is needed in order to develop more effective treatments for conditions and diseases mediated by these TACE-cleaved proteins.

### SUMMARY OF THE INVENTION

The present invention is based upon the discovery that certain membrane-associated proteins are cleaved by metalloproteases such as TACE to generate the soluble form of said proteins.

In a further aspect of the invention, a method is provided for identifying compounds that alter metalloprotease activity comprising
(a) mixing a test compound with cells; and
(b) determining whether the test compound alters the metalloprotease-mediated shedding of protein from said cells.

In another aspect of the invention, a method is provided identifying compounds that inhibit the binding of TACE to metalloprotease-shed membrane-bound polypeptides comprising
(a) mixing a test compound with cells; and
(b) determining whether the test compound inhibits the binding of TACE to said metalloprotease-shed membrane-bound polypeptides.

Further provided by the invention is a method for identifying metalloprotease agonists ir antagonists, comprising the steps of
(a) contacting cells with a compound; and
(b) measuring the LDLr transport activity or the LDLr signaling activity of the cells in the presence and in the absence of the compound;
   wherein the compound is a metalloprotease agonist if its presence decreases the LDLr transport activity or the LDLr signaling activity of the cells, and wherein the compound is a metalloprotease antagonist if its presence increases the LDLr transport activity or the LDLr signaling activity of the cells.

In another aspect of the invention, a method is provided for identifying metalloprotease agonists or antagonists, comprising the steps of
(a) contacting cells with a compound; and
(b) measuring the LR11/SorLA or AXLr signaling activity of the cells in the presence and in the absence of the compound;
   wherein the compound is a metalloprotease antagonist if its presence increases the LR11/SorLA or AXLr signaling activity of the cells, and wherein the compound is a metalloprotease agonist if its presence decreases the LR11/SorLA or AXLr signaling activity of the cells.

The invention also provides a method for increasing shedding of proteins from cells, comprising providing at least one compound selected from the group consisting of TACE polypeptides and agonists of said polypeptides; with a preferred embodiment of the method further comprising increasing said activities in a patient.

Further provided by the invention is a method for decreasing shedding of proteins from cells, comprising providing at least one antagonist of TACE polypeptides; with a preferred embodiment of the method further comprising decreasing said activities in a patient by administering at least one TACE antagonist, and with a further preferred embodiment wherein the antagonist is an antibody or an antisense molecule that inhibits TACE activity.

### BRIEF DESCRIPTION OF THE DRAWINGS

FIG. 1 shows two-dimensional (2D) PAGE gels of proteins from DRM TACE+/+ cells stimulated with PMA for 90 minutes in the absence of the metalloprotease inhibitor IC3. In Panel A, 200 micrograms of supernatant protein, derived from approximately 5×10⁷ cells, were loaded onto the gel. In Panel B, all of the glycoproteins obtained by WGA lectin affinity purification from 5.8 mg of total supernatant proteins (derived from approximately 1.3×10⁹ cells) were N-deglycosylated and loaded onto the gel. Protein assignments were based on database matches to tandem mass spectra (see Table 1). The number of peptides identified from each protein is indicated within parentheses.

FIG. 2. 1D-PAGE gel of supernatant proteins from DRM TACE+/+ cells after WGA lectin affinity purification and N-deglycosylation. DRM TACE+/+ cells were stimulated with PMA for 90 minutes in the presence or absence of the metalloprotease inhibitor IC3. Proteins obtained from equal numbers of cells (approximately 1×10⁹ cells) were loaded in each lane. Matching protein bands were excised from the gel, reduced with DTT, alkylated with either isotopically light (d0) or heavy form (d5) N-ethyl-iodoacetamide, and digested in-gel with trypsin. The peptides from matched bands were combined and analyzed by mass spectrometry. Ion intensity measurements were used for the determination of the d0/d5 ratios, which reflects the relative protein quantities in the mixtures. The staining pattern was reproducible with the exception of a band >200 kDa identified as hybrid receptor SorLA (e.g., FIG. 4). In most cases, the gel staining showed that SorLA was shed in the absence of IC3, and that shedding was inhibited by IC3, indicating that this protein is also a metalloprotease-shed receptor. C# designates an alkylated cysteine. M* indicates methionine sulfoxide. The peptides shown are provided as SEQ ID NOs 84 through 101, starting with the mannose receptor peptide at the top of the figure (LFGFC#PLHFEGSER, SEQ ID NO:84) and continuing sequentially down the figure to the N-glycosidase F peptide (AGWC#PGM*AVPTR, SEQ ID NO:101).

FIG. 3. Expanded section of mass spectra showing examples of ion pairs used in the quantitation of peptide. Mass difference of 5 Da or 10 Da were typically observed for the ion-pairs, depending on the number of cysteines in a given peptide. Panel A: The (M+H₂)⁺² ion of the peptide GC#SFLPDPYQK (SEQ ID NO:126) from saposin (see FIG. 4). Panel B: The (M+H₂)⁺² ion of the peptide C#VPFFYGGC#GGNR (SEQ ID NOs 88, 111, and 117) from amyloid A4 (see FIGS. 2 and 4). C# designates an alkylated cysteine.

FIG. 4. 1D-PAGE gel of supernatant proteins from PMA-stimulated DRM TACE −/− cells and PMA-stimulated DRM TACE −/− cells reconstituted with full-length TACE, following WGA lectin affinity purification and N-deglycosylation. Proteins obtained from equal number of cells (approximately 1×10⁹ cells) were loaded in each lane. Matching protein bands were excised from the gel, reduced with DTT, alkylated with either isotopically light (d0) or heavy form (d5) N-ethyl-iodoacetamide, and digested in-gel with trypsin. Tryptic peptides were combined and analyzed by mass spectrometry. Ion intensity measurements were used for the determination of the d0/d5 ratios, which reflects the relative protein quantities in the two protein mixtures. The protein band marked with ** apparently contained protein(s) that were more abundant in TACE-containing cells in comparison to the control cells. Proteins identified from this band include peroxiredoxin 1 (SWISSPROT P35700), endothelial protein C receptor (SWISSPROT Q64695) and oncostatin M (SWISSPROT S64719). Since none of the cysteine-containing peptides were recovered from these proteins, no quantitative measurement could be derived from the data. C# designates an alkylated cysteine. M* indicates methionine sulfoxide. N(D) indicates the position of a glycosylated asparagine (N) residue that is converted to aspartic acid (D) due to N-glycosidase F treatment. The peptides shown are provided as SEQ ID NOs 102 through 132, starting with the hybrid receptor SorLA peptide at the top of the figure (FMDFVC#K, SEQ ID NO: 102) and continuing sequentially down the figure to the AXLr peptide (C#ELQVQGEPPEVVWLR, SEQ ID NO:132).

FIG. 5. 1D-PAGE gel of supernatant proteins from HMVECs following WGA lectin affinity purification and N-deglycosylation. HMVECs were either untreated or stimulated with cytokines followed by PMA to induce shedding. Proteins obtained from 8×10⁶ cells were loaded in each lane. Matching protein bands were excised from the gel, reduced with DTT, alkylated with either isotopically light (d0) or heavy form (d5) N-ethyl-iodoacetamide, and digested in-gel with trypsin. Tryptic peptides were combined and analyzed by mass spectrometry analysis. Ion intensity measurements were used for the determination of the d0/d5 ratios, which reflect the relative protein quantities in the two protein mixtures. C# designates an alkylated cysteine. The peptides shown are provided as SEQ ID NOs 133 through 136, starting with the Jagged1 peptide C#PEDYEGK (SEQ ID NO:133) and continuing sequentially down to the endothelial cell protein C receptor peptide C#FLGC#ELPPEGSR (SEQ ID NO:136)

FIG. 6. Metalloprotease-mediated shedding of proteins following cell stimulation. A monocyte cell line (DRM) was stimulated using a combination of LPS and PMA, either in the presence or absence of the metalloprotease inhibitor, IC3. Cell supernatants were collected after stimulation, and glycoproteins were isolated using a lectin column. Supernatants from treated and untreated cells were labeled with N-ethyl or d₅-N-ethyl iodoacetamide, respectively. The graph shows the ratio of the amount of peptide detected in supernatants of untreated cells vs. the amount of peptide detected in supernatants of IC3-treated cells. The height of the bars has been normalized by dividing by 0.56, since for most proteins the ion intensity ratios of heavy to light isotopes was found to be, on average, 0.56. Error bars were obtained from cases where multiple peptides were observed for the same protein.

### DETAILED DESCRIPTION OF THE INVENTION

### Identification of Proteins Shed from Cell Membranes

Protein shedding is a post-translational event that is independent of the expression level of messenger RNA (mRNA); hence, screening of protein shedding events requires a proteomic approach. Using a proteomic system for analyzing cell-surface shedding which provides an unbiased means to screen for shed proteins, we identified a number of proteins already known to be shed, thereby validating our methods. In addition, a group of proteins were newly identified as being shed by tumor necrosis factor-alpha converting enzyme (TACE). Two forms of human TACE protein are shown in SEQ ID NOs 1 and 2.

Our methods utilize short-term culture supernatants from cells in which shedding was induced with a phorbol ester (and in some experiments also stimulated with lipopolysaccharide (LPS)) as starting material. Two different cell systems were used: murine Dexter-ras-myc (DRM) monocytic cells and human adult dermal microvascular endothelial cells (HMVEC). Induced shedding events are carried out by one or more metalloproteases, also interchangeably called metalloproteinases, located on the cell surface that can be inhibited by hydroxamic acid compounds such as IC3 (Immunex Compound 3). Relative quantitation was carried out by comparing cell supernatants from cells that were stimulated in the presence or absence of a metalloprotease inhibitor. Proteins that exhibited changes in relative amounts are therefore identified as substrates of inducible metalloprotease sheddases.

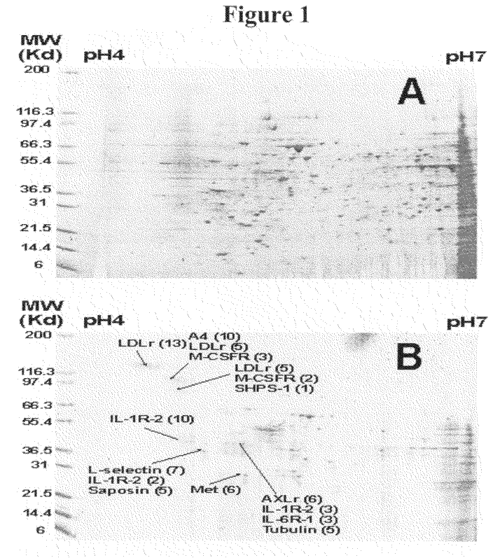

In order to isolate shed proteins, many of which are glycosylated, from cell supernatants, we first utilized a lectin-affinity purification step to isolate glycoproteins. An N-deglycosylation step was subsequently used to reduce the heterogeneity of the protein, which enhanced the resolution on an one-dimensional (1D) sodium dodecyl sulfate (SDS) polyacrylamide gel electrophoresis (PAGE) gel. To quantitatively compare regulated versus constitutive shedding, stable isotope dilution was performed using a novel thiol-alkylating reagent. Global proteome displays on 2D-PAGE may largely be limited to the more abundantly expressed and stable proteins, but, as we describe here and in more detail in Examples 1 through 3 below, applying targeted protein isolation and modification procedures prior to 2D-PAGE yields meaningful results. As demonstrated in FIG. 1, a group of low-abundance proteins, most of which serve as immuno-regulatory proteins, can be effectively displayed by 2D-PAGE if the starting material (short-term cell supernatants in this case) is carefully selected, and the electrophoresis is preceded by lectin affinity fractionation and deglycosylation. Moreover, even 1D-PAGE, a low-cost, reproducible, and rapid method for comparing and characterizing proteins, was found to be effective with these samples. By combining appropriate sample preparation, 1D-PAGE, isotope dilution and mass spectrometry, we have demonstrated a method for comparing the relative abundance of proteins in complex mixtures.

Following isolation from SDS PAGE gels and isotope labeling using the thiol-alkylating agent, protein mixtures were digested with trypsin and the trypsin fragments analyzed by tandem mass spectrometry (MS/MS). Using this isotope dilution and mass spectrometry approach, we have identified several metalloprotease-released proteins, including proteins already known or implicated as metalloprotease-shed proteins. These include amyloid A4 protein, IL-1R-2, IL-6R-1, L-selectin, M-CSFR, SorLA, AXLr and endothelial cell protein C receptor (see references cited above and Xu et al., 2000, J Biol Chem 275: 6038-6044; Hampe et al., 2000, J Cell Sci 113: 4475-4485; Bazil and Strominger, 1991, J Immunol 147: 1567-1574; Nath et al., 2001, J Cell Sci 114: 1213-1220; Reddy et al., 2000, J Biol Chem 275: 14608-14614; and Bellosta et al., 1995, Mol Cell Biol 15: 614-625). Thus, this proteomic technique was validated as a method that can be applied in studies of protein shedding. In addition, this study implicated a number of additional proteins as being shed by metalloproteases, including LDLr, SHPS-1, and Jagged1. TACE was shown to be the responsible protease in the case of the LDLr and some of the previously identified shed proteins (e.g. AXLr and hybrid receptor SorLA) for which the sheddase had not been determined. These metalloprotease-shed proteins, their biological activities, and diseases mediated by them are described in more detail below.

In order to identify metalloprotease-shed proteins, we have also used a new method for making relative quantitative measurements of proteins in complex mixtures . This method was used to study the metalloprotease-mediated shedding of cell surface molecules from a mouse DRM monocyte cell line that had been treated with a phorbol ester (PMA) and lipopolysaccharide (LPS). In addition to the identification of proteins previously determined to be inducibly shed, such as those described in the above paragraphs, three metalloprotease substrates were newly identified as such using this method: CD 18, ICOS ligand, and tumor endothelial marker 7-related (TEM7R).

One common feature of several of these metalloprotease-shed proteins, including LDLr, SorLA, SHPS-1, Jagged1, ICOS ligand, etc. is their ability to transduce signals, associated with ligand binding, into the intracellular environment. In some systems, such as the SorLA homologue in Hydra which binds the head activator (HA) neuropeptide, shedding of the extracellular domain of the membrane-associated protein is believed to act as a negative regulatory control on the protein's signaling activity (Hampe et al., 2000, J Cell Sci 113: 4475-4485). As discussed further below, regulation of the shedding of these extracellular domains by metalloprotease agonists or antagonists provides methods of treating diseases and conditions associated with the signaling activity of these metalloprotease-shed proteins.

### Characteristics of Membrane-Associated Proteins Cleaved by TACE

LDL Receptor.(“LDLr”). LDLr is known as a cell-surface receptor that binds to LDL, the major cholesterol-carrying lipoprotein in plasma, and transports LDL into cells by endocytosis (Brown and Goldstein, 1986, Science 232: 34-47). The amino acid sequence of the Mus musculus LDL receptor is presented as SEQ ID NO:3; another version of the amino acid sequence of the mouse LDL receptor is found at SWISSPROT database accession number P35951. LDL receptors from other mammalian species can be found at the following database accession numbers: human (SWISSPROT P01130), rat (SWISSPROT P35952), Chinese hamster (SWISSPROT P35950), rabbit (SWISSPROT P20063), cow (SWISSPROT P01131), and pig (GenBank AAC39254).

The LDL receptor is a type I membrane protein. Examples of typical structural elements common to members of the LDL receptor family are found in the mouse LDL receptor amino acid sequence, and include a signal sequence (approximately at amino acids 1 through 21 of SEQ ID NO:3), an extracellular domain (approximately at amino acids 22 through 790 of SEQ ID NO:3), a transmembrane domain (approximately at amino acids 791 through 812 of SEQ ID NO:3), and an intracellular domain (approximately at amino acids 813 through 862 of SEQ ID NO:3). The extracellular domain of the murine LDL receptor includes, in N-to-C order, seven LDL receptor class A domains (approximately at amino acids 25 through 314 of SEQ ID NO:3), two EGF-like domains (approximately at amino acids 315 through 394 of SEQ ID NO:3), six LDL receptor class B domains (approximately at amino acids 398 through 657 of SEQ ID NO:3), a third EGF-like domain (approximately at amino acids 663 through 713 of SEQ ID NO:3), and a domain containing sites for the attachment of clustered O-linked oligosaccharides (approximately at amino acids 722 through 770 of SEQ ID NO:3). Each of the LDL receptor class A domains and the EGF-like domains generally includes 3 disulfide bonds, the locations of which are specified within the SWISSPROT accession number P35951 database entry; these disulfide bonds are involved in maintaining the three-dimensional structure of the LDL receptor, such that substitutions of those residues are likely be associated with an altered function or lack of that function for the LDL receptor. The intracellular domain of the LDL receptor includes a domain critical for endocytosis via clathrin-coated pits. The skilled artisan will recognize that the boundaries of the regions of LDLr polypeptides described above are approximate and that the precise boundaries of such domains, as for example the boundaries of the transmembrane region (which can be predicted by using computer programs available for that purpose), can also differ from member to member within the family of LDLr and LDLr-related polypeptides from different species.

LDLr proteins are expressed on a wide variety of cells, and are particularly prevalent on liver and adrenal gland cells (Hussein et al., 1999, Ann Rev Nutr 19: 141-172). Typical biological activities or functions associated with LDLr polypeptides are binding to ligand proteins involved in lipoprotein metabolism such as ApoB and ApoE, and transporting via endocytosis such ligands and any lipids associated with them. A recent report indicates that endocytotic receptors such as LDLr may also be involved in hormone uptake in certain tumor cells, for example breast and prostrate tumor cells (Willnow et al., 1999, Nat Cell Biol 1: E157-E162), and another has identified LDLr as having a role in entry of hepatitis C virus into cells (Agnello et al., 1999, Proc Natl Acad Sci USA 96: 12766-12771). LDLr polypeptides having transport activity bind to extracellular molecules and transport them into the cell via endocytosis. The transport activity is associated with the extracellular domain of LDLr polypeptides, the LDL receptor class A domains, and particularly the fifth of the seven LDL receptor class A domains; endocytosis of LDLr also requires conserved residues (the “NPXY” motif) in the intracellular domain. Thus, for uses requiring LDLr transport activity, preferred LDLr polypeptides include those having the both extracellular domain and the conserved portions of the intracellular domain. When the extracellular domain is separated from the intracellular domain, for example by TACE-mediated cleavage that sheds the LDLr extracellular domain from the cell, the LDLr transport activity is presumably abolished. Another function of the LDLr is related to the intracellular domain, which associates with Disabled1 (Dab1) protein and is predicted to interact through Dab1 with the Abl and Src tyrosine kinase pathways (Gotthardt et al., 2000, JBC Papers in Press, Manuscript M000955200). This signaling activity of LDLr would also presumably be abolished by TACE-mediated shedding of the LDLr extracellular domain.

Due to their role in transporting LDL and other lipids into the cell, conditions that disrupt LDLr lipoprotein transport activity are linked to diseases that share as a common feature failures of lipoprotein and/or cholesterol uptake in their etiology, such as familial hypercholesterolemia, atherosclerosis, dyslipidemia, and heart disease. Additional diseases that may be treated, prevented, or ameliorated by modulating LDLr shedding are aortic aneurisms; arteritis; vascular occlusion, including cerebral artery occlusion; complications of coronary by-pass surgery; ischemia/reperfusion injury; myocarditis, including chronic autoimmune myocarditis and viral myocarditis; heart failure, including chronic heart failure (CHF), cachexia of heart failure; myocardial infarction; restenosis after heart surgery; silent myocardial ischemia; post-implantation complications of left ventricular assist devices; Raynaud's phenomena; thrombophlebitis; vasculitis, including Kawasaki's vasculitis; giant cell arteritis, Wegener's granulomatosis; and Schoenlein-Henoch purpura. Blocking or inhibiting metalloprotease-mediated shedding of LDLr extracellular domains is an aspect of the invention and provides methods for treating or ameliorating these diseases and conditions through the use of inhibitors of metalloproteases such as TACE. Examples of such inhibitors or antagonists are described in more detail below. In instances such as tumors of the prostrate or breast where it is desirable to block endocytic uptake of hormones, or infections of hepatitis C or other Flaviviridae viruses where it is desirable to block entry of virus into cells, methods of treating or ameliorating these conditions comprise increasing the amount or activity of metalloprotease polypeptides such as TACE by providing isolated metalloprotease or TACE polypeptides or active fragments or fusion polypeptides thereof, or by providing compounds (agonists) that activate endogenous or exogenous isolated metalloprotease or TACE polypeptides. Similarly, in conditions where it is preferable to inhibit signaling through LDLr and intracellular proteins such as Dab1, for example to reduce vascular cell proliferation, methods of treating or ameliorating these conditions comprise increasing the amount or activity of metalloprotease polypeptides such as TACE. Preferred methods of administering metalloprotease and/or TACE antagonists or agonists to organisms in need of treatment, such as mammals or most preferably humans, include routes of administration that localize the antagonist or agonist to the site where it is needed, or the use of carriers or targeting agents that direct the antagonist or agonist to the tissues or cells it is desirable to treat.

Additional methods of the invention include assays to identify antagonists or agonists of metalloproteases such as TACE by determining the effect that such compounds have on the shedding of LDLr or on the transport or signaling activities of LDLr. The extracellular domain of LDLr can be detected in supernatants from cell cultures using antibodies specific to extracellular LDLr epitopes in ELISA assays. Additional particularly suitable assays to identify antagonists or agonists of metalloproteases such as TACE are to measure the binding, internalization, and degradation of radioactively labeled LDL using the methods of Goldstein et al., 1983, Methods Enzymol 98: 241-260 and Parise et al., 1999, Human Gene Therapy 10: 1219-1228. Alternatively, endocytosis of DiI-LDL can be measured using the method of Agnello et al., 1999, Proc Natl Acad Sci USA 96: 12766-12771. LDLr signaling activity may be assayed using methods which determine the phosphorylation state of proteins in intracellular signaling pathways such as the Abl and Src tyrosine kinase pathways; such methods can employ phosphorylation-state-specific antibodies to quantitate the specific phosphorylation levels of proteins in the pathway through specific immunoprecipitation of the phosphorylated forms of such proteins. Alternatively, the Ca⁺⁺ flux that is generated by ligand binding to LDLr can be measured using the methods of Allen et al., 1998, J Clin Invest 101: 1064-1075. Preferred antagonists of metalloproteases such as TACE are those that increase LDL uptake, the measure of LDLr transport activity, or peak Ca⁺⁺ flux levels, the measure of LDLr signaling activity, by at least 10% and more preferably by at least 25% as compared to LDL uptake or peak Ca⁺⁺ flux levels in untreated control cells, as measured in one or more of the above assays. Preferred agonists of metalloproteases such as TACE are those that decrease LDL uptake or peak Ca⁺⁺ flux levels by at least 10% and more preferably by at least 25% as compared to LDL uptake or peak Ca⁺⁺ flux levels in untreated control cells, as measured in one or more of the above assays. The change in LDL uptake or in peak Ca⁺⁺ flux levels is measured by dividing the LDL uptake or peak Ca⁺⁺ flux level in treated cells by the LDL uptake or peak Ca⁺⁺ flux level in untreated cells, with a result of 1.10 indicating an increase of 10% in the treated cells. Those of skill in the art will appreciate that other, similar types of assays can be used to measure LDLr transport activity or LDLr signaling activity in assays for TACE agonists or antagonists.

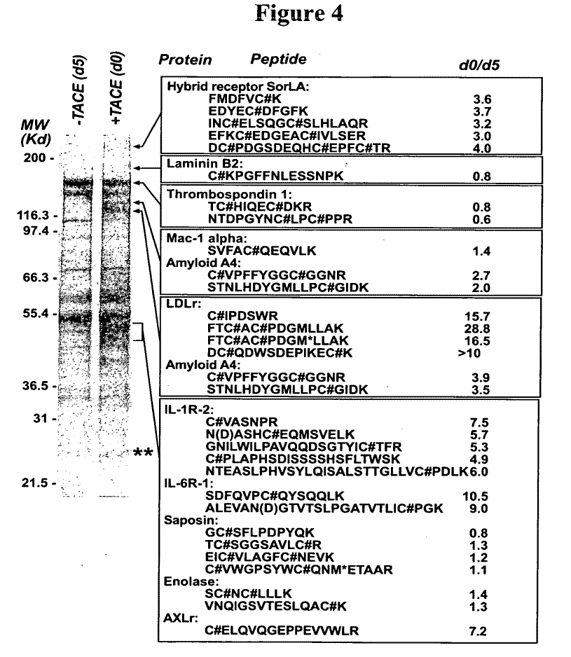

LR11/SorLA. Other LDLr gene family proteins, including LR11/SorLA (see FIG. 4, a shed protein found here to be released by TACE) have been found to engage in a wide range of biological functions (Herz, 2001, Neuron 29: 571-581). The amino acid sequence of the Mus musculus LR11/SorLA protein is presented as SEQ ID NO:4.

LR11/SorLA, like the LDL receptor, is a type I membrane protein. Examples of typical structural elements common to members of the LDL receptor family are found in the mouse LR11/SorLA amino acid sequence, and include a signal sequence (approximately at amino acids 1 through 28 of SEQ ID NO:4), a propeptide believed to be removed by furin (approximately at amino acids 29 through 81 of SEQ ID NO:4), an extracellular domain (approximately at amino acids 82 through 2138 of SEQ ID NO:4), a transmembrane domain (approximately at amino acids 2139 through 2159 of SEQ ID NO:4), and an intracellular domain (approximately at amino acids 2160 through 2215 of SEQ ID NO:4). The extracellular domain of the murine LR11/SorLA protein includes, in N-to-C order, five BNR repeats (approximately at amino acids 136 through 573 of SEQ ID NO:4), a domain having homology to yeast VSP10 protein (approximately at amino acids 369 through 757 of SEQ ID NO:4), a domain containing five YWTD motifs (approximately at amino acids 803 through 977 of SEQ ID NO:4), an EGF-like domain (approximately at amino acids 1026 through 1072 of SEQ ID NO:4), eleven LDL receptor class A domains (approximately at amino acids 1076 through 1551 of SEQ ID NO:4), and six fibronectin type-III domains (approximately at amino acids 1556 through 2116 of SEQ ID NO:4). Each of the LDL receptor class A domains generally includes 3 disulfide bonds, the locations of which are specified within the SWISSPROT accession number 088307 database entry; these disulfide bonds are involved in maintaining the three-dimensional structure of the LR11/SorLA protein, such that substitutions of those residues are likely be associated with an altered function or lack of that function for the LR11/SorLA protein. The intracellular domain of the LR1/SorLA protein includes a domain critical for endocytosis. The skilled artisan will recognize that the boundaries of the regions of LR11/SorLA polypeptides described above are approximate and that the precise boundaries of such domains, as for example the boundaries of the transmembrane region (which can be predicted by using computer programs available for that purpose), can also differ from member to member within the family of LR1/SorLA and LR11/SorLA-related polypeptides from different species.

LR11/SorLA proteins are expressed on a wide variety of cells, and are particularly prevalent on embryonic CNS cells and on adult brain cells such as cerebellar, hippocampal, and dentate gyrus cells, and also in vascular smooth muscle cells. Typical biological activities or functions associated with LR11/SorLA polypeptides are binding to a neuropeptide such as head activator (HA), which is believed to generate an intracellular signal stimulating cell proliferation. LR11/SorLA polypeptides also bind to ligand proteins involved in lipoprotein metabolism such as ApoE, transporting into the cell via endocytosis such ligands and any lipids associated with them. LR11/SorLA expression is upregulated in atherosclerotic lesions and is believed to promote vascular smooth muscle cell proliferation. LR11/SorLA polypeptides having transport activity bind to extracellular molecules and transport them into the cell via endocytosis. The transport activity is associated with the extracellular domain of LR11/SorLA polypeptides and the LDL receptor class A domains; endocytosis of LR11/SorLA also requires conserved residues (the “NPXY” motif) in the intracellular domain. Thus, for uses requiring LR11/SorLA transport activity, preferred LR11/SorLA polypeptides include those having the both extracellular domain and the conserved portions of the intracellular domain. When the extracellular domain is separated from the intracellular domain, for example by TACE-mediated cleavage that sheds the LR11/SorLA extracellular domain from the cell, the LR11/SorLA transport activity is presumably abolished. The signaling activity of LR11/SorLA would also presumably be abolished by TACE-mediated shedding of the LR11/SorLA extracellular domain.

Due to their role in stimulating neural cell proliferation, conditions that disrupt LR11/SorLA signaling activity are linked to diseases that share as a common feature neural cell death or failures of neural cell proliferation in their etiology, such as acute polyneuropathy; anorexia nervosa; Bell's palsy; chronic fatigue syndrome; transmissible dementia, including Creutzfeld-Jacob disease; demyelinating neuropathy; Guillain-Barre syndrome; vertebral disc disease; myasthenia gravis; silent cerebral ischemia; chronic neuronal degeneration; and stroke, including cerebral ischemic diseases. Blocking or inhibiting metalloprotease-mediated shedding of LR1/SorLA extracellular domains is an aspect of the invention and provides methods for treating or ameliorating these diseases and conditions through the use of inhibitors of metalloproteases such as TACE. Examples of such inhibitors or antagonists are described in more detail below. In instances such as LR11/SorLA-mediated proliferation of vascular smooth muscle cells in conditions such as atherosclerosis or restenosis where it is desirable to inhibit such proliferation, methods of treating or ameliorating these conditions comprise increasing the amount or activity of metalloprotease polypeptides such as TACE by providing isolated metalloprotease or TACE polypeptides or active fragments or fusion polypeptides thereof, or by providing compounds (agonists) that activate endogenous or exogenous isolated metalloprotease or TACE polypeptides. Preferred methods of administering metalloprotease and/or TACE antagonists or agonists to organisms in need of treatment, such as mammals or most preferably humans, include routes of administration that localize the antagonist or agonist to the site where it is needed, or the use of carriers or targeting agents that direct the antagonist or agonist to the tissues or cells it is desirable to treat.

Additional methods of the invention include assays to identify antagonists or agonists of metalloproteases such as TACE by determining the effect that such compounds have on the shedding of LR11/SorLA or on the transport or signaling activities of LR1/SorLA. The extracellular domain of LR11/SorLA can be detected in supernatants from cell cultures using antibodies specific to extracellular LR11/SorLA epitopes in ELISA assays. Additional particularly suitable assays to identify antagonists or agonists of metalloproteases such as TACE are to measure HA-induced cell proliferation using the methods of Kayser et al., 1998, Eur J Cell Biol 76: 119-124. Preferred antagonists of metalloproteases such as TACE are those that increase HA-induced cell proliferation, the measure of LR11/SorLA signaling activity, by at least 10% and more preferably by at least 25% as compared to HA-induced cell proliferation of untreated control cells, as measured in any of the above assays. Preferred agonists of metalloproteases such as TACE are those that decrease HA-induced cell proliferation by at least 10% and more preferably by at least 25% as compared to HA-induced cell proliferation of untreated control cells, as measured in any of the above assays. The change in HA-induced cell proliferation is measured by dividing the HA-induced cell proliferation of treated cells by the HA-induced cell proliferation of untreated cells, with a result of 1.10 indicating an increase of 10% in the treated cells. Those of skill in the art will appreciate that other, similar types of assays can be used to measure LR11/SorLA signaling activity in assays for TACE agonists or antagonists.

AXLr. The AXL receptor, also called “UFO oncogene homologue” or “adhesion-related kinase”, is a member of the receptor tyrosine kinase family. The amino acid sequence of the Mus musculus AXLr protein is presented as SEQ ID NO:5; another database entry describing mouse AXLr is SWISSPROT Database accession number Q00993. AXLr is a type I membrane protein. Examples of structural elements found in the mouse AXLr amino acid sequence include a signal sequence (approximately at amino acids 1 through amino acid 18 to 19 of SEQ ID NO:5), an extracellular domain (approximately at amino acids 19 through 445 of SEQ ID NO:5), a transmembrane domain (approximately at amino acids 446 through 466 of SEQ ID NO:5), and an intracellular domain (approximately at amino acids 467 through 888 of SEQ ID NO:5). The extracellular domain of the murine AXLr protein includes, in N-to-C order, two Ig-like C2-type domains (the first approximately at amino acids 43 to 47 through 113 to 118 of SEQ ID NO:5 and the second approximately at amino acids 147 through 206 of SEQ ID NO:5), two fibronectin type-III domains (the first approximately at amino acids 218 to 219 through 315 to 316 of SEQ ID NO:5, and the second approximately at amino acids 320 to 329 through 412 to 417 of SEQ ID NO:5). Each of the Ig-like C2-type domains generally includes a disulfide bond, the locations of which are specified within the SWISSPROT accession number Q00993 database entry; these disulfide bonds are involved in maintaining the three-dimensional structure of the AXLr protein, such that substitutions of those residues are likely be associated with an altered function or lack of that function for the AXLr protein. The intracellular domain of the AXLr protein includes a kinase domain from approximately at amino acids 530 to 532 through 801 to 811 of SEQ ID NO:5). The skilled artisan will recognize that the boundaries of the regions of AXLr polypeptides described above are approximate and that the precise boundaries of such domains, as for example the boundaries of the transmembrane region (which can be predicted by using computer programs available for that purpose), can also differ from member to member within the family of AXLr and AXLr-related polypeptides from different species.

AXLr proteins are expressed during development on a wide variety of cells, and are particularly prevalent on adult connective tissues. AXLr proteins are also expressed on vascular smooth muscle cells and vascular endothelial cells. Typical biological activities or functions associated with AXLr polypeptides are binding to the ligand GAS6, which is believed to generate an intracellular signal stimulating cell proliferation. AXLr expression is upregulated in vascular cells following injury or in response to factors such as thrombin and agniotensin II, and AXLr is believed to promote vascular smooth muscle cell proliferation and the formation of a neointima after injury. The interaction of GAS6 and AXLr has also been found to protect cells from apoptosis, and to induce chemotaxis of vascular smooth muscle cells. When the extracellular ligand-binding domain is separated from the intracellular kinase domain, for example by TACE-mediated cleavage that sheds the AXLr extracellular domain from the cell, the AXLr signaling activity associated with cell proliferation is presumably abolished. Due to their role in stimulating vascular cell proliferation, conditions that disrupt AXLr signaling activity are linked to diseases that share as a common feature cell death or failures of cell proliferation in their etiology. Blocking or inhibiting metalloprotease-mediated shedding of AXLr extracellular domains is an aspect of the invention and provides methods for treating or ameliorating these diseases and conditions, and for treating wounds, through the use of inhibitors of metalloproteases such as TACE. Examples of such inhibitors or antagonists are described in more detail below. In instances such as AXLr-mediated proliferation of vascular smooth muscle cells in conditions such as atherosclerosis or restenosis where it is desirable to inhibit such proliferation, methods of treating or ameliorating these conditions comprise increasing the amount or activity of metalloprotease polypeptides such as TACE by providing isolated metalloprotease or TACE polypeptides or active fragments or fusion polypeptides thereof, or by providing compounds (agonists) that activate endogenous or exogenous isolated metalloprotease or TACE polypeptides. Preferred methods of administering metalloprotease and/or TACE antagonists or agonists to organisms in need of treatment, such as mammals or most preferably humans, include routes of administration that localize the antagonist or agonist to the site where it is needed, or the use of carriers or targeting agents that direct the antagonist or agonist to the tissues or cells it is desirable to treat.

Additional methods of the invention include assays to identify antagonists or agonists of metalloproteases such as TACE by determining the effect that such compounds have on the shedding of AXLr or on the signaling activities of AXLr. The extracellular domain of AXLr can be detected in supernatants from cell cultures using antibodies specific to extracellular AXLr epitopes in ELISA assays. Additional particularly suitable assays to identify antagonists or agonists of metalloproteases such as TACE are to measure AXLr signaling activity directly by measuring AXLr phosphorylation (Nagata et al., 1996, J Biol Chem 271: 30022-30027), or to measure AXLr/GAS6-induced cell proliferation or chemotaxis using the methods of Melaragno et al., 1998, Circ Res 83: 697-704 or of Fridell et al., 1998, J Biol Chem 273: 7123-7126). Preferred antagonists of metalloproteases such as TACE are those that increase AXLr signaling activity by at least 10% and more preferably by at least 25% as compared to the AXLr signaling activity of untreated control cells, as measured in any of the above assays. Preferred agonists of metalloproteases such as TACE are those that decrease AXLr signaling activity by at least 10% and more preferably by at least 25% as compared to the AXLr signaling activity of untreated control cells, as measured in any of the above assays. The change in AXLr signaling activity is measured by dividing the AXLr signaling activity in treated cells by the AXLr signaling activity in untreated cells, with a result of 1.10 indicating an increase of 10% in the treated cells. Those of skill in the art will appreciate that other, similar types of assays can be used to measure AXLr signaling activity in assays for TACE agonists or antagonists.

### Characteristics of Membrane-Associated Proteins Cleaved by Metalloproteases

We have shown that SHPS-1, ICOS Ligand, CD14, CD18, tumor endothelial marker 7-related (TEM7R), and Jagged1 proteins are shed from cells; in the case of SHPS-1, CD14, ICOS Ligand, CD18, and TEM7R by a metalloprotease that is sensitive to the metalloprotease inhibitor IC3; and in the case of Jagged1 in response to cytokine stimulation of cells, presumably as a result of metalloprotease activity. Although TACE has not yet specifically been implicated in the shedding of these proteins, TACE has also not been excluded as the metalloprotease that sheds SHPS-1, ICOS Ligand, CD14, CD18, TEM7R, and/or Jagged1.

SHPS-1. The transmembrane glycoprotein SHPS-1 is a physiological substrate for protein-tyrosine phosphatase SHP-2, and belongs to an inhibitory-receptor superfamily. SHPS-1 is abundantly expressed in macrophages and neural tissue, and has been implicated in regulating intracellular signaling events downstream of receptor protein-tyrosine kinases and integrin-mediated cytoskeletal reorganization and cell motility (Inagaki et al., 2000, EMBO J. 19: 6721-6731); SHPS-1 is also believed to play a role in synaptogenesis. The amino acid sequence of murine SHPS-1 is presented as SEQ ID NO:6; the extracellular domain of SHPS-1 extends approximately from between amino acid 28 and 36 of SEQ ID NO:6 through approximately amino acid 373 of SEQ ID NO:6. Blocking or inhibiting metalloprotease-mediated shedding of SHPS-1 extracellular domains is an aspect of the invention and provides methods for treating or ameliorating diseases and conditions involving synaptogenesis, through the use of inhibitors of metalloproteases such as TACE.

Jagged 1. Jagged 1 is a ligand for the receptor Notch1. Jagged 1 signaling through Notch 1 has been shown to play a role in hematopoiesis. The amino acid sequence of murine Jagged 1 is presented as SEQ ID NO:7; the extracellular domain of Jagged 1 extends approximately from between amino acid 27 and 34 of SEQ ID NO:7 through approximately amino acid 1068 of SEQ ID NO:7. The human Jagged 1 protein has been implicated in Alagille syndrome, a disorder characterized by abnormal liver, heart, skeleton, eye, and face development. An aspect of the invention is the use of metalloproteases and agonists thereof to increase Jagged1 shedding from cells, reducing Jagged 1 signaling through Notch molecules in inhibiting hematopoiesis in the treatment of diseases characterized by overproliferation of hematopoietic cells, such as leukemias and lymphomas (for example, B-cell chronic lymphocytic leukemia, acute myeloid leukemia, Hodgkins lymphoma, and anaplastic large cell lymphoma).

ICOS Ligand. ICOS Ligand (ICOSL) is a glycosylated type I transmembrane protein with amino acid sequence similarity to members of the B7 family, including a V-like and a C-like Ig domain in its extracellular region (Wang et al., 2000, Blood 96: 2808-2813). ICOSL has also been called GL50, B7h, B7-H2, B7RP-1, and LICOS and it exists in two splice forms (the murine ICOSL polypeptides are presented in SEQ ID NOs 8 and 9), which are identical throughout the extracellular and transmembrane region but differ in their intracellular C-termini. ICOSL is expressed on monocytes and macrophages (such as splenic peritoneal macrophages), B cells (such as splenic B cells), endothelial cells (Khayyamian et al., 2002, Proc Natl Acad Sci USA 99: 6198-6203), and on a small subset of CD3+ T cells (such as some unactivated splenic T cells; see Ling et al., 2000, J Immunol 164: 1653-1657). Expression of ICOSL is induced on monocytes by integrin-dependent adhesion to a substrate or by IFN-gamma treatment (Aicher et al., 2000, J Immunol 164: 4689-4696). Treatment of non-lymphoid cells such as 3T3 fibroblasts with TNF or LPS has been reported to induce murine ICOSL RNA expression in these cells; but in contrast, treatment of spleen (lymphoid) cells with LPS resulted in a decrease in ICOSL RNA levels (Swallow et al., 1999, Immunity 11: 423-432). Dendritic cells generated from adherent peripheral blood mononuclear cells (PBMCs) by treatment with GM-CSF and IL-4 express cell surface ICOSL as detected by FACS staining with anti-ICOSL antibodies; this staining is reduced to background levels by treatment of these DCs for 24 hours with LPS (Wang et al., 2000, Blood 96: 2808-2813).

ICOSL interacts with the T cell membrane protein ICOS (“Inducible COStimulator”); ICOS is expressed on activated and resting memory T cells, but not on resting naïve T cells. The ICOS-ICOSL interaction provides a costimulatory signal to ICOS-expressing T cells in conjunction with the stimulatory signal provided to T cells through the T cell receptor. The ICOS-ICOSL costimulatory interaction evidently acts independently of the costimulatory interaction of CD28 and other B7 family members. The effect of the ICOS-ICOSL interaction on T cells has been assessed by treating ICOS-expressing T cells with soluble dimeric forms of ICOSL prepared by attaching the extracellular portion of ICOSL to the constant (Fc) region of an immunoglobulin molecule; ICOSL-Fc is expected to mimic the effect on T-cells of interactions with ICOSL-bearing cells. Conversely, cells expressing ICOSL can be treated with ICOS-Fc to mimic ICOS-dependent signaling. ICOSL-Fc stimulates the proliferation of CD3+ T cells; the secretion by T cells of cytokines including IFN-gamma (Yoshinaga et al., 1999, Nature 402: 827-832), IL-4, and IL-10; and increases the percentages of CD3+ CD25+ or CD3+ CD69+ activated T cells in lymph nodes (Guo et al., 2001, J Immunol 166: 5578-5584). ICOSL-Fc also exacerbates contact hypersensitivity, especially when administered at the challenge stage—this suggests the ICOSL-ICOS interaction has a costimulatory effect on T cells, particularly in the secondary immune response. Constitutively expressed ICOSL-Fc produces lymphoid hyperplasia and stimulation of B cell differentiation (Yoshinaga et al., 1999, Nature 402: 827-832). These results suggest that ICOS engagement by ICOSL-Fc stimulates both Th1 and Th2 responses. ICOS-ICOSL interaction is also involved in allograft transplant rejection (Ozkaynak et al., 2001, Nat Immunol 2: 591-596); clonal expansion of CD8+ T cells in the cytotoxic T lymphocyte response (Liu et al., 2001, J Exp Med 194: 1339-1348); and in the efferent immune response to proteolipid protein (PLP) in the induction of experimental allergic encephalomyelitis (EAE) (Rottman et al., 2001, Nat Immunol 2: 605-611). In mixed lymphocyte reactions, addition of ICOS-Fc inhibits the interaction between antigen-presenting cells (APCs) such as dendritic cells (DCs) and T cells, suggesting that membrane-bound ICOSL on APCs is blocked by ICOS-Fc from interacting with ICOS on T cells (Aicher et al., 2000, J Immunol 164: 4689-4696). Studies of cells and transgenic animals deficient in ICOS have shown that ICOS plays a key role in T cell-mediated stimulation of B cells (for example, in stimulation of IL-4 production), and is critical for germinal center formation (Dong et al., 2001, Nature 409: 97-101; Tafuri et al., 2001, Nature 409: 105-109).

However, T cell costimulation by ICOS-ISOCL interaction in some instances has been shown to have a immunoprotective or immunotolerizing effect. In the earlier, antigen-priming phase of EAE, disruption of ICOS-ISOCL interaction with an anti-ICOS antibody was found to result in more severe disease symptoms (Rottman et al., 2001, Nat Immunol 2: 605-611). ICOS-ICOSL interaction has also been found to be required for the development of regulatory T cells that are involved in regulation of the immune response and in immunotolerance (Akbari et al., 2002, Nat Medicine 8: 1024-1032).

Agonists and antagonists of metalloprotease activity can be used to modulate the metalloprotease-mediated shedding of ICOSL from cells and so modify immune cell function. The effects of agonists and antagonists of metalloprotease activity on T cell costimulation can be measured by treating ICOSL-expressing cells with a metalloprotease agonist or antagonist, then mixing the treated cells with T-cells in the presence of an antigen or antibody that binds to T cell receptor, and measuring the resultant T cell proliferation or cytokine secretion (see FIG. 4 of Yoshinaga et al., 1999, Nature 402: 827-832).

Agonists of metalloprotease function are useful in disrupting or preventing ICOSL-ICOS interactions by increasing the degree to which ICOSL is shed from cell membranes. Use of metalloprotease agonists is expected to reduce the severity of immunological conditions promoted by ICOSL-ICOS interactions, such as contact hypersensitivity, allergic asthma, and transplant rejection.

Provided are methods for using metalloprotease agonists, compositions or combination therapies to increase ICOSL shedding in treatment of immune disorders of the endocrine system. For example, metalloprotease agonists can be used to treat autoimmune diabetes. Other endocrine disorders also are treatable with these compounds, compositions or combination therapies, including Hashimoto's thyroiditis (i.e. autoimmune thyroiditis). Inflammatory conditions of the gastrointestinal system also are treatable by the use of metalloprotease agonists to increase ICOSL shedding, including Crohn's disease; ulcerative colitis; and inflammatory bowel disease. Metalloprotease agonists, compositions, and combination therapies are further used to increase ICOSL shedding in treatment of inflammation of the liver. Inflammatory ocular disorders also are treatable with metalloprotease agonists, compositions or combination therapies. A number of pulmonary disorders also can be treated by increasing ICOSL shedding with metalloprotease agonists, compositions and combination therapies, including allergies, allergic rhinitis, contact dermatitis, atopic dermatitis, and asthma. Various other medical disorders treatable with metalloprotease agonists, compositions and combination therapies include multiple sclerosis and autoimmune hemolytic anemia; dermatological disorders such as psoriasis and contact dermatitis; as well as various autoimmune disorders or diseases associated with hereditary deficiencies.

Other embodiments provide methods for using metalloprotease agonists, compositions or combination therapies to increase ICOSL shedding in the treatment of a variety of rheumatic disorders. These include: adult and juvenile rheumatoid arthritis; systemic lupus erythematosus; gout; osteoarthritis; polymyalgia rheumatica; seronegative spondylarthropathies, including ankylosing spondylitis; and Reiter's disease. Metalloprotease agonists, compositions and combination therapies are used also to treat psoriatic arthritis and chronic Lyme arthritis. Also treatable with these compounds, compositions and combination therapies are Still's disease and uveitis associated with rheumatoid arthritis. In addition, increasing ICOSL shedding with metalloprotease agonists, compositions or combination therapies can be used to treat disorders resulting in inflammation of the voluntary muscle, including dermatomyositis and polymyositis. In addition, metalloprotease agonists, compositions and combinations thereof can be used to increase ICOSL shedding in the treatment of multicentric reticulohistiocytosis, a disease in which joint destruction and papular nodules of the face and hands are associated with excess production of proinflammatory cytokines by multinucleated giant cells that are believed to arise from monocytes and/or macrophages (Gorman et al., 2000, Arthritis and Rheumatism 43: 930-938).

Also treatable by increasing ICOSL shedding with metalloprotease agonists, compositions or combination therapies, are disorders associated with transplantation such as graft-versus-host disease, and complications resulting from solid organ transplantation, including transplantion of heart, liver, lung, skin, kidney, bone marrow, or other organs. Metalloprotease agonists may be administered, for example, to prevent or inhibit the development of bronchiolitis obliterans after lung transplantation, and to prolong graft survival. In addition, metalloprotease agonists, compositions and combination therapies are useful for treating or to suppress the inflammatory response prior, during or after the transfusion of allogeneic red blood cells in cardiac or other surgery, or in treating a traumatic injury to a limb or joint, such as traumatic knee injury.

Various lymphoproliferative disorders, including T-cell-dependent B-cell-mediated diseases, can also be treated by increasing ICOSL shedding with metalloprotease agonists, compositions or combination therapies, and so decreasing costimulation of T cells and T-cell-dependent stimulation of B cells. These disorders include, but are not limited to autoimmune lymphoproliferative syndrome (ALPS), chronic lymphoblastic leukemia, hairy cell leukemia, chronic lymphatic leukemia, peripheral T-cell lymphoma, small lymphocytic lymphoma, mantle cell lymphoma, follicular lymphoma, Burkitt's lymphoma, Epstein-Barr virus-positive T cell lymphoma, histiocytic lymphoma, Hodgkin's disease, diffuse aggressive lymphoma, acute lymphatic leukemias, T gamma lymphoproliferative disease, cutaneous B cell lymphoma, cutaneous T cell lymphoma (i.e., mycosis fungoides), and Sezary syndrome.

Antagonists or inhibitors of metalloprotease function can be used as adjuvants in increasing the immune stimulating response of immunogens, in that inhibition of shedding of ICOSL from APCs is predicted to increase the primary immune response by promoting, increasing, or extending the duration of ICOSL-ICOS interactions. Metalloprotease inhibitors are useful to promote ICOSL-ICOS interactions in the antigen-priming phase of diseases such as EAE, or in the induction of immunotolerance (optionally in combination with IL-10). Further, metalloprotease inhibitors can be used to increase the costimulation of T cells by the ICOS-ICOSL interaction in the secondary immune response. Metalloprotease antagonists, compositions and combination therapies described herein are useful in increasing the immune response to bacterial, viral or protozoal infections; and in reducing or ameliorating complications resulting therefrom. One such disease is Mycoplasma pneumonia. In addition, provided herein is the use of metalloprotease antagonists to treat AIDS and related conditions, such as AIDS dementia complex, AIDS associated wasting, and Kaposi's sarcoma. Provided herein is the use of metalloprotease antagonists for treating protozoal diseases, including malaria and schistosomiasis. Additionally provided is the use of metalloprotease antagonists to treat erythema nodosum leprosum; bacterial or viral meningitis; tuberculosis, including pulmonary tuberculosis; and pneumonitis secondary to a bacterial or viral infection. Provided also herein is the use of metalloprotease antagonists to prepare medicaments for treating louse-bome relapsing fevers, such as that caused by Borrelia recurrentis. Metalloprotease antagonists can also be used to prepare a medicament for treating conditions caused by Herpes viruses, such as herpetic stromal keratitis, corneal lesions, and virus-induced corneal disorders. In addition, metalloprotease agonists or antagonists can be used in treating human papillomavirus infections. Metalloprotease agonists or antagonists are used also to prepare medicaments to treat influenza.

Also provided herein are methods for using metalloprotease agonists or antagonists, compositions or combination therapies to treat various oncologic disorders. For example, metalloprotease agonists or antagonists are used to treat various forms of cancer, including acute myelogenous leukemia, Epstein-Barr virus-positive nasopharyngeal carcinoma, glioma, colon, stomach, prostate, renal cell, cervical and ovarian cancers, lung cancer (SCLC and NSCLC), including cancer-associated cachexia, fatigue, asthenia, paraneoplastic syndrome of cachexia and hypercalcemia. Additional diseases treatable with metalloprotease agonists or antagonists, compositions or combination therapies are solid tumors, including sarcoma, osteosarcoma, and carcinoma, such as adenocarcinoma (for example, breast cancer) and squamous cell carcinoma. In addition, the subject compounds, compositions or combination therapies are useful for treating leukemia, including acute myelogenous leukemia, chronic or acute lymphoblastic leukemia and hairy cell leukemia. Other malignancies with invasive metastatic potential can be treated with metalloprotease agonists or antagonists, compositions and combination therapies, including multiple myeloma. A combination of at least one metalloprotease agonists or antagonists and one or more other anti-angiogenesis factors may be used to treat solid tumors, thereby reducing the vascularization that nourishes the tumor tissue. Suitable anti-angiogenic factors for such combination therapies include IL-8 inhibitors, angiostatin, endostatin, kringle 5, inhibitors of vascular endothelial growth factor (such as antibodies against vascular endothelial growth factor), angiopoietin-2 or other antagonists of angiopoietin-1, antagonists of platelet-activating factor and antagonists of basic fibroblast growth factor.

CD14. CD14 (SEQ ID NO:10), the receptor for lipopolysaccharide (LPS) and other glycosylated ligands, is a GPI-linked protein on the exterior of the cell membrane. As it is GPI-linked, it is believed that the signal generated by LPS binding to CD14 is transmitted into the cell through an association of CD14 with a transmembrane polypeptide such as CD11c and/or CD18 integrin, or a member of the Toll-like receptor family such as Toll-Like Receptor 4 (TLR4) (Triantafilou M. and Triantafilou K., 2002, Trends Immunol 23: 301-301; Pfeiffer A. et al., 2001, Eur J Immunol 31: 3153-3164). Soluble CD14 in serum has been used as a positively correlated marker for sepsis and disease susceptibility, and may have a role in transport of phospholipids in and out of cells (Sugiyama and Wright, 2001, J Immunol 166: 826-831). Soluble CD14 may be released from cells by a combination of two mechanisms: secretion without the formation of a GPI linkage, and proteolytic shedding (Bufler et al., 1995, Eur J Immunol 25: 604-610). Publications describing the shedding of GPI-linked CD14 have suggested that something other than phosphatidylinositol-phospholipase C (PI-PLC), for example, was involved in shedding CD14 from cell membranes, because soluble CD14 from serum or PMA-induced cells was slightly smaller than CD14 removed from cells by PI-PLC, and that a serine protease—such as human leukocyte elastase (HLE)—was responsible for the shedding (Bazil and Strominger, 1991, J Immunology 147: 1567-1574; Le-Barillec et al., 1999, J. Clin. Invest 103: 1039-1046). However, our present results (see Example 4 below) indicate that an IC3-dependent mechanism, presumably the action of a metalloprotease, is at least a component of shedding of CD14 induced by PMA and LPS.

Another aspect of the invention is the use of metalloprotease antagonists to reduce the shedding of CD14 from cells, prolonging the response of cells such as monocytes and macrophages to lipopolysaccharide (LPS) and other glycosylated ligands, and/or to increasing the sensitivity of CD14-expressing cells to such ligands. Conversely, as signaling through CD14 promotes inflammatory responses, there is a use of metalloproteases or agonists thereof to increase shedding of CD14, reducing the inflammatory response.

CD18. CD18 is the beta2 integrin; murine CD18 is presented as SEQ ID NO:11. CD18 associates with a variety of alpha integrins to form the beta2 family of integrins, which includes LFA-1, Mac-1/CR3 (complement receptor 3), and CR4 (complement receptor 4). CR3 is involved in phagocytosis. LFA-1 and Mac-1 share ICAM-1 as a ligand, and CD18-containing integrins are involved in T cell adhesion and in adhesion of neutrophils on vascular endothelium, leading to transendothelial migration. Administration of metalloproteinases and agonists thereof to increase the shedding of CD18 from the surface of cells, such as endothelial cells or immune cells expressing CR3 or CR4, is useful in reducing inflammatory responses and the interaction of immune cells such as neutrophils with endothelial cells such as vascular endothelial cells.

TEM7R. TEM7R (tumor endothelial marker 7-related) is a transmembrane protein identified as a marker present on human and murine endothelial colon tumor cells, but not on the corresponding normal colon endothelial cells (Carson-Walter et al., 2001, Cancer Research 61: 6649-6655). TEM7R polypeptide (murine TEM7R is presented as SEQ ID NO:12) comprises a plexin-like domain in its extracellular region. Plexins are semaphorin receptors and are involved in neural development. Our present results indicate that murine TMEM7R is shed in an IC3-dependent manner from DRM monocytes upon stimulation by PMA and LPS (see Example 4 below). Administration of metalloproteinases and agonists thereof to increase the shedding of TEM7R from the surface of tumor cells, such as colon carcinoma cells or other endothelial tumor cells, is useful in disrupting interactions between such tumor cells and cells expressing TEM7R binding partners such as semaphorins.

### Additional Assays of Metallonrotease-Shed Polylevtide Activities

Purified metalloprotease-shed polypeptides of the invention (including polypeptides, polypeptides, fragments, variants, oligomers, and other forms) are useful in a variety of assays. For example, the metalloprotease-shed polypeptides of the present invention can be used to identify agonists or inhibitors of TACE binding to such polypeptides, agonists or inhibitors which can also be used to modulate lipid uptake or cell proliferation.

Yeast Two-Hybrid or “Interaction Trap” Assays. Where a TACE polypeptide binds or potentially binds to a metalloprotease-shed polypeptide, the nucleic acid encoding the metalloprotease-shed polypeptide can be used in interaction trap assays (such as, for example, that described in Gyuris et al., Cell 75:791-803 (1993)) to identify agonists or inhibitors of the binding interaction, such as peptide or small molecule inhibitors or agonists of the binding interaction.

Cell Proliferation, Cell Death, Cell Differentiation, and Cell Adhesion Assays. A soluble form of a metalloprotease-shed polypeptide of the present invention may exhibit cytokine, cell proliferation (either inducing or inhibiting), or cell differentiation (either inducing or inhibiting) activity, or may induce production of other cytokines in certain cell populations. The activity of a soluble form of a polypeptide of the present invention is evidenced by any one of a number of routine cell proliferation assays for cell lines including, without limitation, 32D, DA2, DA1G, T10, B9, B9/11, BaF3, MC9/G, M+ (preB M+), 2E8, RB5, DA1, 123, T1165, HT2, CTLL2, TF-1, Mo7e and CMK.

### Producing Metalloprotease-Shed Polypeptides

Metalloprotease-shed polypeptides can be isolated from naturally occurring sources, or have the same structure as naturally occurring metalloprotease-shed polypeptides, or can be produced to have structures that differ from naturally occurring metalloprotease-shed polypeptides. Methods of producing polypeptides by culturing recombinant cells comprising polypeptide-encoding nucleic acids are well known in the art. Polypeptides derived from any metalloprotease-shed polypeptide by any type of alteration (for example, but not limited to, insertions, deletions, or substitutions of amino acids; changes in the state of glycosylation of the polypeptide; refolding or isomerization to change its three-dimensional structure or self-association state; and changes to its association with other polypeptides or molecules), but which are capable of being shed from cells by metalloproteases, are also metalloprotease-shed polypeptides. Therefore, the polypeptides provided by the invention include polypeptides characterized by amino acid sequences similar to those of the metalloprotease-shed polypeptides described herein, but into which modifications are naturally provided or deliberately engineered.

The present invention provides both full-length and mature forms of metalloprotease-shed polypeptides. Full-length polypeptides are those having the complete primary amino acid sequence of the polypeptide as initially translated. The amino acid sequences of full-length polypeptides can be obtained, for example, by translation of the complete open reading frame (“ORF”) of a cDNA molecule. Several full-length polypeptides can be encoded by a single genetic locus if multiple mRNA forms are produced from that locus by alternative splicing or by the use of multiple translation initiation sites. The “mature form” of a polypeptide refers to a polypeptide that has undergone post-translational processing steps such as cleavage of the signal sequence or proteolytic cleavage to remove a prodomain. Multiple mature forms of a particular full-length polypeptide may be produced, for example by cleavage of the signal sequence at multiple sites, or by differential regulation of proteases that cleave the polypeptide. A polypeptide preparation can therefore include a mixture of polypeptide molecules having different N-terminal amino acids. The mature form(s) of such polypeptide can be obtained by expression, in a suitable mammalian cell or other host cell, of a nucleic acid molecule that encodes the full-length polypeptide. Also encompassed within the invention are variations attributable to differences in proteolysis in different types of host cells, such as differences in the position of cleavage of the signal peptide, or differences in the N- or C-termini due to proteolytic removal of one or more terminal amino acids from the polypeptide (generally from 1-5 terminal amino acids). The sequence of the mature form of the polypeptide may be determinable from the amino acid sequence of the full-length form, through identification of signal sequences or protease cleavage sites. The metalloprotease-shed polypeptides of the invention also include those that result from post-transcriptional or post-translational processing events such as alternate mRNA processing which can yield a truncated but biologically active polypeptide, for example, a naturally occurring soluble form of the polypeptide.

The invention further includes metalloprotease-shed polypeptides with or without associated native-pattern glycosylation. Polypeptides expressed in yeast or mammalian expression systems (e.g., COS-1 or CHO cells) can be similar to or significantly different from a native polypeptide in molecular weight and glycosylation pattern, depending upon the choice of expression system. Expression of polypeptides of the invention in bacterial expression systems, such as E. coli, provides non-glycosylated molecules. Further, a given preparation can include multiple differentially glycosylated species of the polypeptide. Glycosyl groups can be removed through conventional methods, in particular those utilizing glycopeptidase. In general, glycosylated polypeptides of the invention can be incubated with a molar excess of glycopeptidase (Boehringer Mannheim).

Species homologues of metalloprotease-shed polypeptides and of nucleic acids encoding them are also provided by the present invention. As used herein, a “species homologue” is a polypeptide or nucleic acid with a different species of origin from that of a given polypeptide or nucleic acid, but with significant sequence similarity to the given polypeptide or nucleic acid, as determined by those of skill in the art. Species homologues can be isolated and identified by making suitable probes or primers from polynucleotides encoding the amino acid sequences provided herein and screening a suitable nucleic acid source from the desired species. The invention also encompasses allelic variants of metalloprotease-shed polypeptides and nucleic acids encoding them; that is, naturally-occurring alternative forms of such polypeptides and nucleic acids in which differences in amino acid or nucleotide sequence are attributable to genetic polymorphism (allelic variation among individuals within a population).

Fragments of the metalloprotease-shed polypeptides of the present invention are encompassed by the present invention and can be in linear form or cyclized using known methods, for example, as described in Saragovi et al., Bio/Technology 10, 773-778 (1992) and in McDowell et al., J. Amer. Chem. Soc. 114 9245-9253 (1992). Polypeptides and polypeptide fragments of the present invention, and nucleic acids encoding them, include polypeptides and nucleic acids with amino acid or nucleotide sequence lengths that are at least 25% (more preferably at least 50%, or at least 60%, or at least 70%, and most preferably at least 80%) of the length of a metalloprotease-shed polypeptide and have at least 60% sequence identity (more preferably at least 70%, at least 75%, at least 80%, at least 85%, at least 90%, at least 95%, at least 97.5%, or at least 99%, and most preferably at least 99.5%) with that metalloprotease-shed polypeptide or encoding nucleic acid, where sequence identity is determined by comparing the amino acid sequences of the polypeptides when aligned so as to maximize overlap and identity while minimizing sequence gaps. Also included in the present invention are polypeptides and polypeptide fragments, and nucleic acids encoding them, that contain or encode a segment preferably comprising at least 8, or at least 10, or preferably at least 15, or more preferably at least 20, or still more preferably at least 30, or most preferably at least 40 contiguous amino acids. Such polypeptides and polypeptide fragments may also contain a segment that shares at least 70% sequence identity (more preferably at least 70%, at least 75%, at least 80%, at least 85%, at least 90%, at least 95%, at least 97.5%, or at least 99%, and most preferably at least 99.5%) with any such segment of any metalloprotease-shed polypeptide, where sequence identity is determined by comparing the amino acid sequences of the polypeptides when aligned so as to maximize overlap and identity while minimizing sequence gaps. The percent identity of two amino acid or two nucleic acid sequences can be determined by visual inspection and mathematical calculation, or more preferably, the comparison is done by comparing sequence information using a computer program. An exemplary, preferred computer program is the Genetics Computer Group (GCG; Madison, Wis.) Wisconsin package version 10.0 program, ‘GAP’ (Devereux et al., 1984, Nucl. Acids Res. 12: 387). The preferred default parameters for the ‘GAP’ program includes: (1) The GCG implementation of a unary comparison matrix (containing a value of 1 for identities and 0 for non-identities) for nucleotides, and the weighted amino acid comparison matrix of Gribskov and Burgess, Nucl. Acids Res. 14:6745, 1986, as described by Schwartz and Dayhoff, eds., Atlas of Polypeptide Sequence and Structure, National Biomedical Research Foundation, pp. 353-358, 1979; or other comparable comparison matrices; (2) a penalty of 30 for each gap and an additional penalty of 1 for each symbol in each gap for amino acid sequences, or penalty of 50 for each gap and an additional penalty of 3 for each symbol in each gap for nucleotide sequences; (3) no penalty for end gaps; and (4) no maximum penalty for long gaps. Other programs used by those skilled in the art of sequence comparison can also be used, such as, for example, the BLASTN program version 2.0.9, available for use via the National Library of Medicine website www.ncbi.nlm.nih.gov/gorf/wblast2.cgi, or the UW-BLAST 2.0 algorithm. Standard default parameter settings for UW-BLAST 2.0 are described at the following Internet site: sapiens.wustl.edu/blast/blast/#Features. In addition, the BLAST algorithm uses the BLOSUM62 amino acid scoring matix, and optional parameters that can be used are as follows: (A) inclusion of a filter to mask segments of the query sequence that have low compositional complexity (as determined by the SEG program of Wootton and Federhen (Computers and Chemistry, 1993); also see Wootton and Federhen, 1996, Analysis of compositionally biased regions in sequence databases, Methods Enzymol. 266: 554-71) or segments consisting of short-periodicity internal repeats (as determined by the XNU program of Claverie and States (Computers and Chemistry, 1993)), and (B) a statistical significance threshold for reporting matches against database sequences, or E-score (the expected probability of matches being found merely by chance, according to the stochastic model of Karlin and Altschul (1990); if the statistical significance ascribed to a match is greater than this E-score threshold, the match will not be reported.); preferred E-score threshold values are 0.5, or in order of increasing preference, 0.25, 0.1, 0.05, 0.01, 0.001, 0.0001, 1e-5, 1e-10, 1e-15, 1e-20, 1e-25, 1e-30, 1e-40, 1e-50, 1e-75, or 1e-100.

The present invention also provides for soluble forms of metalloprotease-shed polypeptides comprising certain fragments or domains of these polypeptides, and particularly those comprising the extracellular domain or one or more fragments of the extracellular domain. Soluble polypeptides are polypeptides that are capable of being secreted from the cells in which they are expressed. In such forms part or all of the intracellular and transmembrane domains of the polypeptide are deleted such that the polypeptide is fully secreted from the cell in which it is expressed. The intracellular and transmembrane domains of polypeptides of the invention can be identified in accordance with known techniques for determination of such domains from sequence information. Soluble metalloprotease-shed polypeptides also include those polypeptides which include part of the transmembrane region, provided that the soluble metalloprotease-shed polypeptide is capable of being secreted from a cell, and preferably retains metalloprotease-shed polypeptide activity. Soluble metalloprotease-shed polypeptides further include oligomers or fusion polypeptides comprising the extracellular portion of at least one metalloprotease-shed polypeptide, and fragments of any of these polypeptides that have metalloprotease-shed polypeptide activity. A secreted soluble polypeptide can be identified (and distinguished from its non-soluble membrane-bound counterparts) by separating intact cells which express the desired polypeptide from the culture medium, e.g., by centrifugation, and assaying the medium (supernatant) for the presence of the desired polypeptide. The presence of the desired polypeptide in the medium indicates that the polypeptide was secreted from the cells and thus is a soluble form of the polypeptide. The use of soluble forms of metalloprotease-shed polypeptides is advantageous for many applications. Purification of the polypeptides from recombinant host cells is facilitated, since the soluble polypeptides are secreted from the cells. Moreover, soluble polypeptides are generally more suitable than membrane-bound forms for parenteral administration and for many enzymatic procedures.

In another aspect of the invention, preferred polypeptides comprise various combinations of metalloprotease-shed polypeptide domains, such as the extracellular domain and the intracellular domain, or fragments thereof. Accordingly, polypeptides of the present invention and nucleic acids encoding them include those comprising or encoding two or more copies of a domain such as a portion of the extracellular domain, two or more copies of a domain such as a portion of the intracellular domain, or at least one copy of each domain, and these domains can be presented in any order within such polypeptides.

Further modifications in the peptide or DNA sequences can be made by those skilled in the art using known techniques. Modifications of interest in the polypeptide sequences can include the alteration, substitution, replacement, insertion or deletion of a selected amino acid. For example, one or more of the cysteine residues can be deleted or replaced with another amino acid to alter the conformation of the molecule, an alteration which may involve preventing formation of incorrect intramolecular disulfide bridges upon folding or renaturation. Techniques for such alteration, substitution, replacement, insertion or deletion are well known to those skilled in the art (see, e.g., U.S. Pat. No. 4,518,584). As another example, N-glycosylation sites in the polypeptide extracellular domain can be modified to preclude glycosylation, allowing expression of a reduced carbohydrate analog in mammalian and yeast expression systems. N-glycosylation sites in eukaryotic polypeptides are characterized by an amino acid triplet Asn-X-Y, wherein X is any amino acid except Pro and Y is Ser or Thr. Appropriate substitutions, additions, or deletions to the nucleotide sequence encoding these triplets will result in prevention of attachment of carbohydrate residues at the Asn side chain. Alteration of a single nucleotide, chosen so that Asn is replaced by a different amino acid, for example, is sufficient to inactivate an N-glycosylation site. Alternatively, the Ser or Thr can by replaced with another amino acid, such as Ala. Known procedures for inactivating N-glycosylation sites in polypeptides include those described in U.S. Pat. No. 5,071,972 and EP 276,846. Additional variants within the scope of the invention include polypeptides that can be modified to create derivatives thereof by forming covalent or aggregative conjugates with other chemical moieties, such as glycosyl groups, lipids, phosphate, acetyl groups and the like. Covalent derivatives can be prepared by linking the chemical moieties to functional groups on amino acid side chains or at the N-terminus or C-terminus of a polypeptide. Conjugates comprising diagnostic (detectable) or therapeutic agents attached thereto are contemplated herein. Preferably, such alteration, substitution, replacement, insertion or deletion retains the desired activity of the polypeptide or a substantial equivalent thereof. One example is a variant that binds with essentially the same binding affinity as does the native form. Binding affinity can be measured by conventional procedures, e.g., as described in U.S. Pat. No. 5,512,457 and as set forth herein.

Other derivatives include covalent or aggregative conjugates of the polypeptides with other polypeptides or polypeptides, such as by synthesis in recombinant culture as N-terminal or C-terminal fusions. Examples of fusion polypeptides are discussed below in connection with oligomers. Further, fusion polypeptides can comprise peptides added to facilitate purification and identification. Such peptides include, for example, poly-His or the antigenic identification peptides described in U.S. Pat. No. 5,011,912 and in Hopp et al., Bio/Technology 6:1204, 1988. One such peptide is the FLAG® peptide, which is highly antigenic and provides an epitope reversibly bound by a specific monoclonal antibody, enabling rapid assay and facile purification of expressed recombinant polypeptide. A murine hybridoma designated 4E11 produces a monoclonal antibody that binds the FLAG® peptide in the presence of certain divalent metal cations, as described in U.S. Pat. No. 5,011,912. The 4E11 hybridoma cell line has been deposited with the American Type Culture Collection under accession no. HB 9259. Monoclonal antibodies that bind the FLAG® peptide are available from Eastman Kodak Co., Scientific Imaging Systems Division, New Haven, Conn.

Encompassed by the invention are oligomers or fusion polypeptides that contain a metalloprotease-shed polypeptide, one or more fragments of metalloprotease-shed polypeptides, or any of the derivative or variant forms of metalloprotease-shed polypeptides as disclosed herein. In particular embodiments, the oligomers comprise soluble metalloprotease-shed polypeptides. Oligomers can be in the form of covalently linked or non-covalently-linked multimers, including dimers, trimers, or higher oligomers. In one aspect of the invention, the oligomers maintain the binding ability of the polypeptide components and provide therefor, bivalent, trivalent, etc., binding sites. In an alternative embodiment the invention is directed to oligomers comprising multiple metalloprotease-shed polypeptides joined via covalent or non-covalent interactions between peptide moieties fused to the polypeptides, such peptides having the property of promoting oligomerization. Leucine zippers and certain polypeptides derived from antibodies are among the peptides that can promote oligomerization of the polypeptides attached thereto, as described in more detail below.

In embodiments where variants of the metalloprotease-shed polypeptides are constructed to include a membrane-spanning domain, they will form a Type I membrane polypeptide. Membrane-spanning metalloprotease-shed polypeptides can be fused with extracellular domains of receptor polypeptides for which the ligand is known. Such fusion polypeptides can then be manipulated to control the intracellular signaling pathways triggered by the membrane-spanning metalloprotease-shed polypeptide. metalloprotease-shed polypeptides that span the cell membrane can also be fused with agonists or antagonists of cell-surface receptors, or cellular adhesion molecules to further modulate metalloprotease-shed intracellular effects. In another aspect of the present invention, interleukins can be situated between the preferred metalloprotease-shed polypeptide fragment and other fusion polypeptide domains.

Immunoglobulin-based Oligomers. The polypeptides of the invention or fragments thereof can be fused to molecules such as immunoglobulins for many purposes, including increasing the valency of polypeptide binding sites. For example, fragments of a metalloprotease-shed polypeptide can be fused directly or through linker sequences to the Fc portion of an immunoglobulin. For a bivalent form of the polypeptide, such a fusion could be to the Fc portion of an IgG molecule. Other immunoglobulin isotypes can also be used to generate such fusions. For example, a polypeptide-IgM fusion would generate a decavalent form of the polypeptide of the invention. The term “Fc polypeptide” as used herein includes native and mutein forms of polypeptides made up of the Fc region of an antibody comprising any or all of the CH domains of the Fc region. Truncated forms of such polypeptides containing the hinge region that promotes dimerization are also included. Preferred Fc polypeptides comprise an Fc polypeptide derived from a human IgG1 antibody. As one alternative, an oligomer is prepared using polypeptides derived from immunoglobulins. Preparation of fusion polypeptides comprising certain heterologous polypeptides fused to various portions of antibody-derived polypeptides (including the Fc domain) has been described, e.g., by Ashkenazi et al. (PNAS USA 88:10535, 1991); Byrn et al. (Nature 344:677, 1990); and Hollenbaugh and Aruffo (“Construction of Immunoglobulin Fusion Polypeptides”, in Current Protocols in Immunology, Suppl. 4, pages 10.19.1-10.19.11, 1992). Methods for preparation and use of immunoglobulin-based oligomers are well known in the art. One embodiment of the present invention is directed to a dimer comprising two fusion polypeptides created by fusing a polypeptide of the invention to an Fc polypeptide derived from an antibody. A gene fusion encoding the polypeptide/Fc fusion polypeptide is inserted into an appropriate expression vector. Polypeptide/Fc fusion polypeptides are expressed in host cells transformed with the recombinant expression vector, and allowed to assemble much like antibody molecules, whereupon interchain disulfide bonds form between the Fc moieties to yield divalent molecules. One suitable Fc polypeptide, described in PCT application WO 93/10151, is a single chain polypeptide extending from the N-terminal hinge region to the native C-terminus of the Fc region of a human IgG1 antibody. Another useful Fc polypeptide is the Fc mutein described in U.S. Pat. No. 5,457,035 and in Baum et al., (EMBO J. 13:3992-4001, 1994). The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors. The above-described fusion polypeptides comprising Fc moieties (and oligomers formed therefrom) offer the advantage of facile purification by affinity chromatography over Polypeptide A or Polypeptide G columns. In other embodiments, the polypeptides of the invention can be substituted for the variable portion of an antibody heavy or light chain. If fusion polypeptides are made with both heavy and light chains of an antibody, it is possible to form an oligomer with as many as four metalloprotease-shed extracellular regions.

Peptide-linker Based Oligomers. Alternatively, the oligomer is a fusion polypeptide comprising multiple metalloprotease-shed polypeptides, with or without peptide linkers (spacer peptides). Among the suitable peptide linkers are those described in U.S. Pat. Nos. 4,751,180 and 4,935,233. A DNA sequence encoding a desired peptide linker can be inserted between, and in the same reading frame as, the DNA sequences of the invention, using any suitable conventional technique. For example, a chemically synthesized oligonucleotide encoding the linker can be ligated between the sequences. In particular embodiments, a fusion polypeptide comprises from two to four soluble metalloprotease-shed polypeptides, separated by peptide linkers. Suitable peptide linkers, their combination with other polypeptides, and their use are well known by those skilled in the art.

Leucine-Zippers. Another method for preparing the oligomers of the invention involves use of a leucine zipper. Leucine zipper domains are peptides that promote oligomerization of the polypeptides in which they are found. Leucine zippers were originally identified in several DNA-binding polypeptides (Landschulz et al., Science 240:1759, 1988), and have since been found in a variety of different polypeptides. Among the known leucine zippers are naturally occurring peptides and derivatives thereof that dimerize or trimerize. The zipper domain (also referred to herein as an oligomerizing, or oligomer-forming, domain) comprises a repetitive heptad repeat, often with four or five leucine residues interspersed with other amino acids. Use of leucine zippers and preparation of oligomers using leucine zippers are well known in the art.

Other fragments and derivatives of the sequences of polypeptides which would be expected to retain polypeptide activity in whole or in part and may thus be useful for screening or other immunological methodologies can also be made by those skilled in the art given the disclosures herein. Such modifications are believed to be encompassed by the present invention.

### Nucleic Acids Encoding Metalloprotease-Shed Polypeptides

Encompassed within the invention are methods employing metalloprotease-shed polypeptides produced using nucleic acids encoding said polypeptides. These nucleic acids can be identified in several ways, including isolation of genomic or cDNA molecules from a suitable source. Nucleotide sequences corresponding to the amino acid sequences described herein, to be used as probes or primers for the isolation of nucleic acids or as query sequences for database searches, can be obtained by “back-translation” from the amino acid sequences, or by identification of regions of amino acid identity with polypeptides for which the coding DNA sequence has been identified. The well-known polymerase chain reaction (PCR) procedure can be employed to isolate and amplify a DNA sequence encoding a metalloprotease-shed polypeptide or a desired combination of metalloprotease-shed polypeptide fragments. Oligonucleotides that define the desired termini of the combination of DNA fragments are employed as 5′ and 3′ primers. The oligonucleotides can additionally contain recognition sites for restriction endonucleases, to facilitate insertion of the amplified combination of DNA fragments into an expression vector. PCR techniques are described in Saiki et al., Science 239:487 (1988); Recombinant DNA Methodology, Wu et al., eds., Academic Press, Inc., San Diego (1989), pp. 189-196; and PCR Protocols: A Guide to Methods and Applications, Innis et. al., eds., Academic Press, Inc. (1990).

Nucleic acid molecules of the invention include DNA and RNA in both single-stranded and double-stranded form, as well as the corresponding complementary sequences. DNA includes, for example, cDNA, genomic DNA, chemically synthesized DNA, DNA amplified by PCR, and combinations thereof. The nucleic acid molecules of the invention include full-length genes or cDNA molecules as well as a combination of fragments thereof. The nucleic acids of the invention are preferentially derived from human sources, but the invention includes those derived from non-human species, as well.

An “isolated nucleic acid” is a nucleic acid that has been separated from adjacent genetic sequences present in the genome of the organism from which the nucleic acid was isolated, in the case of nucleic acids isolated from naturally-occurring sources. In the case of nucleic acids synthesized enzymatically from a template or chemically, such as PCR products, cDNA molecules, or oligonucleotides for example, it is understood that the nucleic acids resulting from such processes are isolated nucleic acids. An isolated nucleic acid molecule refers to a nucleic acid molecule in the form of a separate fragment or as a component of a larger nucleic acid construct. In one preferred embodiment, the nucleic acids are substantially free from contaminating endogenous material. The nucleic acid molecule has preferably been derived from DNA or RNA isolated at least once in substantially pure form and in a quantity or concentration enabling identification, manipulation, and recovery of its component nucleotide sequences by standard biochemical methods (such as those outlined in Sambrook et al., Molecular Cloning. A Laboratory Manual, 2nd ed., Cold Spring Harbor Laboratory, Cold Spring Harbor, N.Y. (1989)). Such sequences are preferably provided and/or constructed in the form of an open reading frame uninterrupted by internal non-translated sequences, or introns, that are typically present in eukaryotic genes. Sequences of non-translated DNA can be present 5′ or 3′ from an open reading frame, where the same do not interfere with manipulation or expression of the coding region.

The present invention also includes nucleic acids that hybridize under moderately stringent conditions, and more preferably highly stringent conditions, to nucleic acids encoding metalloprotease-shed polypeptides described herein. The basic parameters affecting the choice of hybridization conditions and guidance for devising suitable conditions are set forth by Sambrook, Fritsch, and Maniatis (1989, Molecular Cloning: A Laboratory Manual, Cold Spring Harbor Laboratory Press, Cold Spring Harbor, N.Y., chapters 9 and 11; and Current Protocols in Molecular Biology, 1995, Ausubel et al., eds., John Wiley & Sons, Inc., sections 2.10 and 6.3-6.4), and can be readily determined by those having ordinary skill in the art based on, for example, the length and/or base composition of the DNA. One way of achieving moderately stringent conditions involves the use of a prewashing solution containing 5×SSC, 0.5% SDS, 1.0 mM EDTA (pH 8.0), hybridization buffer of about 50% formamide, 6×SSC, and a hybridization temperature of about 55 degrees C. (or other similar hybridization solutions, such as one containing about 50% formamide, with a hybridization temperature of about 42 degrees C.), and washing conditions of about 60 degrees C., in 0.5×SSC, 0.1% SDS. Generally, highly stringent conditions are defined as hybridization conditions as above, but with washing at approximately 68 degrees C., 0.2×SSC, 0.1% SDS. SSPE (1×SSPE is 0.15M NaCl, 10 mM NaH.sub.2 PO.sub.4, and 1.25 mM EDTA, pH 7.4) can be substituted for SSC (1×SSC is 0.15M NaCl and 15 mM sodium citrate) in the hybridization and wash buffers; washes are performed for 15 minutes after hybridization is complete. It should be understood that the wash temperature and wash salt concentration can be adjusted as necessary to achieve a desired degree of stringency by applying the basic principles that govern hybridization reactions and duplex stability, as known to those skilled in the art and described further below (see, e.g., Sambrook et al., 1989). When hybridizing a nucleic acid to a target nucleic acid of unknown sequence, the hybrid length is assumed to be that of the hybridizing nucleic acid. When nucleic acids of known sequence are hybridized, the hybrid length can be determined by aligning the sequences of the nucleic acids and identifying the region or regions of optimal sequence complementarity. The hybridization temperature for hybrids anticipated to be less than 50 base pairs in length should be 5 to 110.degrees C. less than the melting temperature (Tm) of the hybrid, where Tm is determined according to the following equations. For hybrids less than 18 base pairs in length, Tm (degrees C.)=2(# of A+T bases)+4(# of #G+C bases). For hybrids above 18 base pairs in length, Tm (degrees C.)=81.5+16.6(log₁₀ [Na⁺])+0.41(% G+C)−(600/N), where N is the number of bases in the hybrid, and [Na⁺] is the concentration of sodium ions in the hybridization buffer ([Na⁺] for 1×SSC=0.165M). Preferably, each such hybridizing nucleic acid has a length that is at least 15 nucleotides (or more preferably at least 18 nucleotides, or at least 20 nucleotides, or at least 25 nucleotides, or at least 30 nucleotides, or at least 40 nucleotides, or most preferably at least 50 nucleotides), or at least 25% (more preferably at least 50%, or at least 60%, or at least 70%, and most preferably at least 80%) of the length of the nucleic acid of the present invention to which it hybridizes, and has at least 60% sequence identity (more preferably at least 70%, at least 75%, at least 80%, at least 85%, at least 90%, at least 95%, at least 97.5%, or at least 99%, and most preferably at least 99.5%) with the nucleic acid of the present invention to which it hybridizes, where sequence identity is determined by comparing the sequences of the hybridizing nucleic acids when aligned so as to maximize overlap and identity while minimizing sequence gaps as described in more detail above.

The present invention also provides genes corresponding to the nucleic acid sequences disclosed herein. “Corresponding genes” or “corresponding genomic nucleic acids” are the regions of the genome that are transcribed to produce the mRNAs from which cDNA nucleic acid sequences are derived and can include contiguous regions of the genome necessary for the regulated expression of such genes. Corresponding genes can therefore include but are not limited to coding sequences, 5′ and 3′ untranslated regions, alternatively spliced exons, introns, promoters, enhancers, and silencer or suppressor elements. Corresponding genomic nucleic acids can include 10000 basepairs (more preferably, 5000 basepairs, still more preferably, 2500 basepairs, and most preferably, 1000 basepairs) of genomic nucleic acid sequence upstream of the first nucleotide of the genomic sequence corresponding to the initiation codon of the metalloprotease-shed coding sequence, and 10000 basepairs (more preferably, 5000 basepairs, still more preferably, 2500 basepairs, and most preferably, 1000 basepairs) of genomic nucleic acid sequence downstream of the last nucleotide of the genomic sequence corresponding to the termination codon of the metalloprotease-shed coding sequence. The corresponding genes or genomic nucleic acids can be isolated in accordance with known methods using the sequence information disclosed herein. Such methods include the preparation of probes or primers from the disclosed sequence information for identification and/or amplification of genes in appropriate genomic libraries or other sources of genomic materials. An “isolated gene” or “an isolated genomic nucleic acid” is a genomic nucleic acid that has been separated from the adjacent genomic sequences present in the genome of the organism from which the genomic nucleic acid was isolated.

### Antagonists and Agonists of Metalloprotease Polypeptides

The invention encompasses new uses for antagonists and agonists of metalloproteases, and particularly new uses for antagonists and agonists of the metalloprotease TACE. TACE is referred to herein as an exemplary metalloprotease involved in the shedding of extracellular polypeptide domains (“ectodomains”) from cells, but those of skill in the art will recognize that the description and examples herein can also be applied to other metalloproteases or “sheddases” that shed polypeptide ectodomains from cells.

Any method which neutralizes TACE polypeptides or inhibits expression of the TACE genes (either transcription or translation) can be used to reduce the biological activities of TACE polypeptides.

A class of TACE antagonists are the hydroxamate inhibitors of the metalloprotease catalytic domain of TACE. Examples of such inhibitors are IC3 and ortho-sulfonamide heteroarly hydroxamic acids such as those described in U.S. Pat. No. 6,162,821, which is incorporated by reference herein. Additional TACE antagonists are described in U.S. Pat. Nos. 6,441,023; 6,228,869; 6,197,795; 6,197,791; 6,162,814; 5,977,408; and 5,962,481; all of which are incorporated by reference herein.

In particular embodiments, antagonists inhibit the binding of at least one TACE polypeptide to cells, thereby inhibiting biological activities induced by the binding of those TACE polypeptides to the cells. In certain other embodiments of the invention, antagonists can be designed to reduce the level of endogenous TACE gene expression, e.g., using well-known antisense or ribozyme approaches to inhibit or prevent translation of TACE mRNA transcripts; triple helix approaches to inhibit transcription of TACE family genes; or targeted homologous recombination to inactivate or “knock out” the TACE genes or their endogenous promoters or enhancer elements. Such antisense, ribozyme, and triple helix antagonists can be designed to reduce or inhibit either unimpaired, or if appropriate, mutant TACE gene activity. Techniques for the production and use of such molecules are well known to those of skill in the art. Peptide agonists and antagonists of metalloproteases can also be identified and utilized (see, for example, WO 00/24782 and WO 01/83525, which are incorporated by reference herein). Such peptide agonists and antagonists can be selected in a process comprising one or more techniques selected from yeast-based screening, rational design, protein structural analysis, screening of a phage display library, an E. coli display library, a ribosomal library, an RNA-peptide library, and a chemical peptide library. In further aspects of the invention, the peptide agonists and antagonists are selected from a plurality of randomized peptides.

Antisense RNA and DNA molecules act to directly block the translation of mRNA by hybridizing to targeted mRNA and preventing polypeptide translation. Antisense approaches involve the design of oligonucleotides (either DNA or RNA) that are complementary to a TACE mRNA. The antisense oligonucleotides will bind to the complementary target gene mRNA transcripts and prevent translation. Absolute complementarity, although preferred, is not required. A sequence “complementary” to a portion of a nucleic acid, as referred to herein, means a sequence having sufficient complementarity to be able to hybridize with the nucleic acid, forming a stable duplex (or triplex, as appropriate). In the case of double-stranded antisense nucleic acids, a single strand of the duplex DNA can thus be tested, or triplex formation can be assayed. The ability to hybridize will depend on both the degree of complementarity and the length of the antisense nucleic acid. Preferred oligonucleotides are complementary to the 5′ end of the message, e.g., the 5′ untranslated sequence up to and including the AUG initiation codon. However, oligonucleotides complementary to the 5′- or 3′-non-translated, non-coding regions of the TACE gene transcript, or to the coding regions, could be used in an antisense approach to inhibit translation of endogenous TACE mRNA. Antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides. The oligonucleotides can be DNA or RNA or chimeric mixtures or derivatives or modified versions thereof, single-stranded or double-stranded. Chimeric oligonucleotides, oligonucleosides, or mixed oligonucleotides/oligonucleosides of the invention can be of several different types. These include a first type wherein the “gap” segment of nucleotides is positioned between 5′ and 3′ “wing” segments of linked nucleosides and a second “open end” type wherein the “gap” segment is located at either the 3′ or the 5′ terminus of the oligomeric compound (see, e.g., U.S. Pat. No. 5,985,664). Oligonucleotides of the first type are also known in the art as “gapmers” or gapped oligonucleotides. Oligonucleotides of the second type are also known in the art as “hemimers” or “wingmers”. The oligonucleotide can be modified at the base moiety, sugar moiety, or phosphate backbone, for example, to improve stability of the molecule, hybridization, etc. The oligonucleotide can include other appended groups such as peptides (e.g., for targeting host cell receptors in vivo), or agents facilitating transport across the cell membrane (see, e.g., Letsinger et al., 1989, Proc Natl Acad Sci U.S.A. 86: 6553-6556; Lemaitre et al., 1987, Proc Natl Acad Sci 84: 648-652; PCT Publication No. WO88/09810), or hybridization-triggered cleavage agents or intercalating agents. (See, e.g., Zon, 1988, Pharm. Res. 5: 539-549). The antisense molecules should be delivered to cells which express the TACE transcript in vivo. A number of methods have been developed for delivering antisense DNA or RNA to cells; e.g., antisense molecules can be injected directly into the tissue or cell derivation site, or modified antisense molecules, designed to target the desired cells (e.g., antisense linked to peptides or antibodies that specifically bind receptors or antigens expressed on the target cell surface) can be administered systemically. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous TACE gene transcripts and thereby prevent translation of the TACE mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells.

Ribozyme molecules designed to catalytically cleave TACE mRNA transcripts can also be used to prevent translation of TACE mRNA and expression of TACE polypeptides. (See, e.g., PCT International Publication WO90/11364 and U.S. Pat. No. 5,824,519). The ribozymes that can be used in the present invention include hammerhead ribozymes (Haseloff and Gerlach, 1988, Nature, 334:585-591), RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (International Patent Application No. WO 88/04300; Been and Cech, 1986, Cell, 47:207-216). As in the antisense approach, the ribozymes can be composed of modified oligonucleotides (e.g. for improved stability, targeting, etc.) and should be delivered to cells which express the TACE polypeptide in vivo. A preferred method of delivery involves using a DNA construct “encoding” the ribozyme under the control of a strong constitutive pol III or pol II promoter, so that transfected cells will produce sufficient quantities of the ribozyme to destroy endogenous TACE messages and inhibit translation. Because ribozymes, unlike antisense molecules, are catalytic, a lower intracellular concentration is required for efficiency.

Alternatively, endogenous TACE gene expression can be reduced by targeting deoxyribonucleotide sequences complementary to the regulatory region of the target gene (i.e., the target gene promoter and/or enhancers) to form triple helical structures that prevent transcription of the target TACE gene. (See generally, Helene, 1991, Anticancer Drug Des., 6(6), 569-584; Helene, et al., 1992, Ann. N.Y. Acad. Sci., 660, 27-36; and Maher, 1992, Bioassays 14(12), 807-815).

Anti-sense RNA and DNA, ribozyme, and triple helix molecules of the invention can be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Oligonucleotides can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al., 1988, Nucl. Acids Res. 16:3209. Methylphbsphonate oligonucleotides can be prepared by use of controlled pore glass polymer supports (Sarin et al., 1988, Proc. Natl. Acad. Sci. U.S.A. 85:7448-7451). Alternatively, RNA molecules can be generated by in vitro and in vivo transcription of DNA sequences encoding the antisense RNA molecule. Such DNA sequences can be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Endogenous target gene expression can also be reduced by inactivating or “knocking out” the target gene or its promoter using targeted homologous recombination (e.g., see Smithies, et al., 1985, Nature 317, 230-234; Thomas and Capecchi, 1987, Cell 51, 503-512; Thompson, et al., 1989, Cell 5, 313-321). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra), or in model organisms such as Caenorhabditis elegans where the “RNA interference” (“RNAi”) technique (Grishok, Tabara, and Mello, 2000, Genetic requirements for inheritance of RNAi in C. elegans, Science 287 (5462): 2494-2497), or the introduction of transgenes (Dernburg et al., 2000, Transgene-mediated cosuppression in the C. elegans germ line, Genes Dev. 14 (13): 1578-1583) are used to inhibit the expression of specific target genes. However this approach can be adapted for use in humans provided the recombinant DNA constructs are directly administered or targeted to the required site in vivo using appropriate vectors such as viral vectors.

Organisms that have enhanced, reduced, or modified expression of the gene(s) corresponding to the nucleic acid sequences disclosed herein are provided. The desired change in gene expression can be achieved through the use of antisense nucleic acids or ribozymes that bind and/or cleave the mRNA transcribed from the gene (Albert and Morris, 1994, Trends Pharmacol. Sci. 15(7): 250-254; Lavarosky et al., 1997, Biochem. Mol. Med. 62(1): 11-22; and Hampel, 1998, Prog. Nucleic Acid Res. Mol. Biol. 58: 1-39). Transgenic animals that have multiple copies of the gene(s) corresponding to the nucleic acid sequences disclosed herein, preferably produced by transformation of cells with genetic constructs that are stably maintained within the transformed cells and their progeny, are provided. Transgenic animals that have modified genetic control regions that increase or reduce gene expression levels, or that change temporal or spatial patterns of gene expression, are also provided (see European Patent No. 0 649 464 B1). In addition, organisms are provided in which the gene(s) corresponding to the nucleic acid sequences disclosed herein have been partially or completely inactivated, through insertion of extraneous sequences into the corresponding gene(s) or through deletion of all or part of the corresponding gene(s). Partial or complete gene inactivation can be accomplished through insertion, preferably followed by imprecise excision, of transposable elements (Plasterk, 1992, Bioessays 14(9): 629-633; Zwaal et al., 1993, Proc. Natl. Acad. Sci. USA 90(16): 7431-7435; Clark et al., 1994, Proc. Natl. Acad. Sci. USA 91(2): 719-722), or through homologous recombination, preferably detected by positive/negative genetic selection strategies (Mansour et al., 1988, Nature 336: 348-352; U.S. Pat. Nos. 5,464,764; 5,487,992; 5,627,059; 5,631,153; 5,614,396; 5,616,491; and 5,679,523). These organisms with altered gene expression are preferably eukaryotes and more preferably are mammals. Such organisms are useful for the development of non-human models for the study of disorders involving the corresponding gene(s), and for the development of assay systems for the identification of molecules that interact with the polypeptide product(s) of the corresponding gene(s).

Also encompassed within the invention are TACE polypeptide variants with partner binding sites that have been altered in conformation so that (1) the TACE variant will still bind to its partner(s), but a specified small molecule will fit into the altered binding site and block that interaction, or (2) the TACE variant will no longer bind to its partner(s) unless a specified small molecule is present (see for example Bishop et al., 2000, Nature 407: 395-401). Nucleic acids encoding such altered TACE polypeptides can be introduced into organisms according to methods described herein, and can replace the endogenous nucleic acid sequences encoding the corresponding TACE polypeptide. Such methods allow for the interaction of a particular TACE polypeptide with its binding partners to be regulated by administration of a small molecule compound to an organism, either systemically or in a localized manner.

The TACE polypeptides themselves can also be employed in inhibiting a biological activity of TACE in in vitro or in vivo procedures. Encompassed within the invention are domains of TACE polypeptides that act as “dominant negative” inhibitors of native TACE polypeptide function when expressed as fragments or as components of fusion polypeptides. For example, a purified polypeptide domain of the present invention can be used to inhibit binding of TACE polypeptides to endogenous binding partners. Such use effectively would block TACE polypeptide interactions and inhibit TACE polypeptide activities. Furthermore, antibodies which bind to TACE polypeptides often inhibit TACE polypeptide activity and act as antagonists. For example, antibodies that specifically recognize one or more epitopes of TACE polypeptides, or epitopes of conserved variants of TACE polypeptides, or peptide fragments of the TACE polypeptide can be used in the invention to inhibit TACE polypeptide activity. Such antibodies include but are not limited to polyclonal antibodies, monoclonal antibodies (mAbs), humanized or chimeric antibodies, single chain antibodies, Fab fragments, F(ab′)2 fragments, fragments produced by a Fab expression library, anti-idiotypic (anti-Id) antibodies, and epitope-binding fragments of any of the above. Alternatively, purified and modified TACE polypeptides of the present invention can be administered to modulate interactions between TACE polypeptides and TACE binding partners that are not membrane-bound. Such an approach will allow an alternative method for the modification of TACE-influenced bioactivity.

In an alternative aspect, the invention further encompasses the use of agonists of metalloprotease polypeptide activity to treat or ameliorate the symptoms of a disease for which increased activity of a metalloprotease such as TACE is beneficial. Any method which increases or enhances the activity of metalloprotease polypeptides such as TACE or increases expression of the metalloprotease gene(s) (either transcription or translation) can be used to agonize the biological activities of metalloproteases. In a preferred aspect, the invention entails administering compositions comprising an TACE nucleic acid or an TACE polypeptide to cells in vitro, to cells ex vivo, to cells in vivo, and/or to a multicellular organism such as a vertebrate or mammal. Preferred therapeutic forms of TACE are soluble forms, as described above. In still another aspect of the invention, the compositions comprise administering a TACE-encoding nucleic acid for expression of a TACE polypeptide in a host organism for treatment of disease. Particularly preferred in this regard is expression in a human patient for treatment of a dysfunction associated with aberrant (e.g., decreased) endogenous activity of a TACE family polypeptide. Furthermore, the invention encompasses the administration to cells and/or organisms of compounds found to increase the endogenous activity of TACE polypeptides. One example of compounds that increase TACE polypeptide activity are agonistic antibodies, preferably monoclonal antibodies, that bind to TACE polypeptides or binding partners, which may increase TACE polypeptide activity by causing constitutive intracellular signaling (or “ligand mimicking”), or by preventing the binding of a native inhibitor of TACE polypeptide activity.

Antibodies to Metalloproteases such as TACE Polypeptides

Antibodies that are immunoreactive with the polypeptides of the invention are provided herein. Such antibodies specifically bind to the polypeptides via the antigen-binding sites of the antibody (as opposed to non-specific binding). In the present invention, specifically binding antibodies are those that will specifically recognize and bind with metalloprotease polypeptides such as TACE polypeptides, homologues, and variants, but not with other molecules. In one preferred embodiment, the antibodies are specific for the polypeptides of the present invention and do not cross-react with other polypeptides. In this manner, the TACE polypeptides, fragments, variants, fusion polypeptides, etc., as set forth above can be employed as “immunogens” in producing antibodies immunoreactive therewith.

More specifically, the polypeptides, fragment, variants, fusion polypeptides, etc. contain antigenic determinants or epitopes that elicit the formation of antibodies. These antigenic determinants or epitopes can be either linear or conformational (discontinuous). Linear epitopes are composed of a single section of amino acids of the polypeptide, while conformational or discontinuous epitopes are composed of amino acids sections from different regions of the polypeptide chain that are brought into close proximity upon polypeptide folding (Janeway and Travers, Immuno Biology 3:9 (Garland Publishing Inc., 2nd ed. 1996)). Because folded polypeptides have complex surfaces, the number of epitopes available is quite numerous; however, due to the conformation of the polypeptide and steric hindrances, the number of antibodies that actually bind to the epitopes is less than the number of available epitopes (Janeway and Travers, Immuno Biology 2:14 (Garland Publishing Inc., 2nd ed. 1996)). Epitopes can be identified by any of the methods known in the art. Thus, one aspect of the present invention relates to the antigenic epitopes of the polypeptides of the invention. Such epitopes are useful for raising antibodies, in particular monoclonal antibodies, as described in more detail below. Additionally, epitopes from the polypeptides of the invention can be used as research reagents, in assays, and to purify specific binding antibodies from substances such as polyclonal sera or supernatants from cultured hybridomas. Such epitopes or variants thereof can be produced using techniques well known in the art such as solid-phase synthesis, chemical or enzymatic cleavage of a polypeptide, or using recombinant DNA technology.

As to the antibodies that can be elicited by the epitopes of the polypeptides of the invention, whether the epitopes have been isolated or remain part of the polypeptides, both polyclonal and monoclonal antibodies can be prepared by conventional techniques. See, for example, Monoclonal Antibodies, Hybridomas. A New Dimension in Biological Analyses, Kennet et al. (eds.), Plenum Press, New York (1980); and Antibodies. A Laboratory Manual, Harlow and Land (eds.), Cold Spring Harbor Laboratory Press, Cold Spring Harbor, N.Y., (1988); Kohler and Milstein, (U.S. Pat. No. 4,376,110); the human B-cell hybridoma technique (Kozbor et al., 1984, J. Immunol. 133:3001-3005; Cole et al., 1983, Proc. Natl. Acad. Sci. USA 80:2026-2030); and the EBV-hybridoma technique (Cole et al., 1985, Monoclonal Antibodies And Cancer Therapy, Alan R. Liss, Inc., pp. 77-96). Hybridoma cell lines that produce monoclonal antibodies specific for the polypeptides of the invention are also contemplated herein. Such hybridomas can be produced and identified by conventional techniques. The hybridoma producing the mAb of this invention can be cultivated in vitro or in vivo. Production of high titers of mAbs in vivo makes this the presently preferred method of production. One method for producing such a hybridoma cell line comprises immunizing an animal with a polypeptide; harvesting spleen cells from the immunized animal; fusing said spleen cells to a myeloma cell line, thereby generating hybridoma cells; and identifying a hybridoma cell line that produces a monoclonal antibody that binds the polypeptide. For the production of antibodies, various host animals can be immunized by injection with one or more of the following: a TACE polypeptide, a fragment of a TACE polypeptide, a functional equivalent of a TACE polypeptide, or a mutant form of a TACE polypeptide. Such host animals can include but are not limited to rabbits, guinea pigs, mice, and rats. Various adjuvants can be used to increase the immunologic response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjutants such as BCG (bacille Calmette-Guerin) and Corynebacterium parvum. The monoclonal antibodies can be recovered by conventional techniques. Such monoclonal antibodies can be of any immunoglobulin class including IgG, IgM, IgE, IgA, IgD and any subclass thereof.

In addition, techniques developed for the production of “chimeric antibodies” (Takeda et al., 1985, Nature, 314: 452-454; Morrison et al., 1984, Proc Natl Acad Sci USA 81: 6851-6855; Boulianne et al., 1984, Nature 312: 643-646; Neuberger et al., 1985, Nature 314: 268-270) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a porcine mAb and a human immunoglobulin constant region. The monoclonal antibodies of the present invention also include humanized versions of murine monoclonal antibodies. Such humanized antibodies can be prepared by known techniques and offer the advantage of reduced immunogenicity when the antibodies are administered to humans. In one embodiment, a humanized monoclonal antibody comprises the variable region of a murine antibody (or just the antigen binding site thereof) and a constant region derived from a human antibody. Alternatively, a humanized antibody fragment can comprise the antigen binding site of a murine monoclonal antibody and a variable region fragment (lacking the antigen-binding site) derived from a human antibody. Procedures for the production of chimeric and further engineered monoclonal antibodies include those described in Riechmann et al. (Nature 332:323, 1988), Liu et al. (PNAS 84:3439, 1987), Larrick et al. (Bio/Technology 7:934, 1989), and Winter and Harris (TIPS 14:139, Can, 1993). Useful techniques for humanizing antibodies are also discussed in U.S. Pat. No. 6,054,297. Procedures to generate antibodies transgenically can be found in GB 2,272,440, U.S. Pat. Nos. 5,569,825 and 5,545,806, and related patents. Preferably, for use in humans, the antibodies are human or humanized; techniques for creating such human or humanized antibodies are also well known and are commercially available from, for example, Medarex Inc. (Princeton, N.J.) and Abgenix Inc. (Fremont, Calif.). In another preferred embodiment, fully human antibodies for use in humans are produced by screening a library of human antibody variable domains using either phage display methods (Vaughan et al., 1998, Nat. Biotechnol. 16(6): 535-539; and U.S. Pat. No. 5,969,108), ribosome display methods (Schaffitzel et al., 1999, J Immunol Methods 231(1-2): 119-135), or mRNA display methods (Wilson et al., 2001, Proc Natl Acad Sci USA 98(7): 3750-3755).

Antigen-binding antibody fragments that recognize specific epitopes can be generated by known techniques. For example, such fragments include but are not limited to: the F(ab′)2 fragments which can be produced by pepsin digestion of the antibody molecule and the Fab fragments which can be generated by reducing the disulfide bridges of the (ab′)2 fragments. Alternatively, Fab expression libraries can be constructed (Huse et al., 1989, Science, 246:1275-1281) to allow rapid and easy identification of monoclonal Fab fragments with the desired specificity. Techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can also be adapted to produce single chain antibodies against TACE gene products. Single chain antibodies are formed by linking the heavy and light chain fragments of the Fv region via an amino acid bridge, resulting in a single chain polypeptide. Such single chain antibodies can also be useful intracellularly (i.e., as ‘intrabodies), for example as described by Marasco et al. (J. Immunol. Methods 231:223-238, 1999) for genetic therapy in HIV infection. In addition, antibodies to the TACE polypeptide can, in turn, be utilized to generate anti-idiotype antibodies that “mimic” the TACE polypeptide and that may bind to the TACE polypeptide's binding partners using techniques well known to those skilled in the art. (See, e.g., Greenspan & Bona, 1993, FASEB J 7(5):437-444; and Nissinoff, 1991, J. Immunol. 147(8):2429-2438).

Antibodies that are immunoreactive with the polypeptides of the invention include bispecific antibodies (i.e., antibodies that are immunoreactive with the polypeptides of the invention via a first antigen binding domain, and also immunoreactive with a different polypeptide via a second antigen binding domain). A variety of bispecific antibodies have been prepared, and found useful both in vitro and in vivo (see, for example, U.S. Pat. No. 5,807,706; and Cao and Suresh, 1998, Bioconjugate Chem 9: 635-644). Numerous methods of preparing bispecific antibodies are known in the art, including the use of hybrid-hybridomas such as quadromas, which are formed by fusing two differed hybridomas, and triomas, which are formed by fusing a hybridoma with a lymphocyte (Milstein and Cuello, 1983, Nature 305: 537-540; U.S. Pat. No. 4,474,893; and U.S. Pat. No. 6,106,833). U.S. Pat. No. 6,060,285 discloses a process for the production of bispecific antibodies in which at least the genes for the light chain and the variable portion of the heavy chain of an antibody having a first specificity are transfected into a hybridoma cell secreting an antibody having a second specificity. Chemical coupling of antibody fragments has also been used to prepare antigen-binding molecules having specificity for two different antigens (Brennan et al., 1985, Science 229: 81-83; Glennie et al., J. Immunol., 1987, 139:2367-2375; and U.S. Pat. No. 6,010,902). Bispecific antibodies can also be produced via recombinant means, for example, by using. the leucine zipper moieties from the Fos and Jun proteins (which preferentially form heterodimers) as described by Kostelny et al. (J. Immunol. 148:1547-4553; 1992). U.S. Pat. No. 5,582,996 discloses the use of complementary interactive domains (such as leucine zipper moieties or other lock and key interactive domain structures) to facilitate heterodimer formation in the production of bispecific antibodies. Tetravalent, bispecific molecules can be prepared by fusion of DNA encoding the heavy chain of an F(ab′)2 fragment of an antibody with either DNA encoding the heavy chain of a second F(ab′)2 molecule (in which the CH1 domain is replaced by a CH3 domain), or with DNA encoding a single chain FV fragment of an antibody, as described in U.S. Pat. No. 5,959,083. Expression of the resultant fusion genes in mammalian cells, together with the genes for the corresponding light chains, yields tetravalent bispecific molecules having specificity for selected antigens. Bispecific antibodies can also be produced as described in U.S. Pat. No. 5,807,706. Generally, the method involves introducing a protuberance (constructed by replacing small amino acid side chains with larger side chains) at the interface of a first polypeptide and a corresponding cavity (prepared by replacing large amino acid side chains with smaller ones) in the interface of a second polypeptide. Moreover, single-chain variable fragments (sFvs) have been prepared by covalently joining two variable domains; the resulting antibody fragments can form dimers or trimers, depending on the length of a flexible linker between the two variable domains (Kortt et al., 1997, Protein Engineering 10:423-433).

Screening procedures by which such antibodies can be identified are well known, and can involve immunoaffinity chromatography, for example. Antibodies can be screened for agonistic (i.e., ligand-mimicking) properties. Such antibodies, upon binding to cell surface TACE, induce biological effects (e.g., transduction of biological signals) similar to the biological effects induced when the TACE binding partner binds to cell surface TACE. Agonistic antibodies can be used to induce TACE-mediated cell stimulatory pathways or intercellular communication. Bispecific antibodies can be identified by screening with two separate assays, or with an assay wherein the bispecific antibody serves as a bridge between the first antigen and the second antigen (the latter is coupled to a detectable moiety). Bispecific antibodies that bind TACE polypeptides of the invention via a first antigen-binding domain and a metalloprotease-shed polypeptide via a second antigen-binding domain will be useful in diagnostic applications and in treating conditions through modulation of TACE activity.

Those antibodies that can block binding of the TACE polypeptides of the invention to binding partners for TACE can be used to inhibit TACE-mediated intercellular communication or cell stimulation that results from such binding. Such blocking antibodies can be identified using any suitable assay procedure, such as by testing antibodies for the ability to inhibit binding of TACE to certain cells expressing an TACE binding partner. Alternatively, blocking antibodies can be identified in assays for the ability to inhibit a biological effect that results from binding of soluble TACE to target cells. Antibodies can be assayed for the ability to inhibit TACE binding partner-mediated cell stimulatory pathways, for example. Such an antibody can be employed in an in vitro procedure, or administered in vivo to inhibit a biological activity mediated by the entity that generated the antibody. Disorders caused or exacerbated (directly or indirectly) by the interaction of TACE with cell surface binding partner receptor thus can be treated. A therapeutic method involves in vivo administration of a blocking antibody to a mammal in an amount effective in inhibiting TACE binding partner-mediated biological activity. Monoclonal antibodies are generally preferred for use in such therapeutic methods. In one embodiment, an antigen-binding antibody fragment is employed. Compositions comprising an antibody that is directed against TACE, and a physiologically acceptable diluent, excipient, or carrier, are provided herein. Suitable components of such compositions are as described below for compositions containing TACE polypeptides.

Also provided herein are conjugates comprising a detectable (e.g., diagnostic) or therapeutic agent, attached to the antibody. Examples of such agents are presented above. The conjugates find use in in vitro or in vivo procedures. The antibodies of the invention can also be used in assays to detect the presence of the polypeptides or fragments of the invention, either in vitro or in vivo. The antibodies also can be employed in purifying polypeptides or fragments of the invention by immunoaffinity chromatography.

### Administration of Metalloprotease Polypeptides Agonists, and Antagonists Thereof

This invention provides compounds, compositions, and methods for treating a patient, preferably a mammalian patient, and most preferably a human patient, who is suffering from a medical disorder. For purposes of this disclosure, the terms “illness,” “disease,” “medical condition,” “abnormal condition” and the like are used interchangeably with the term “medical disorder.” The terms “treat”, “treating”, and “treatment” used herein includes curative, preventative (e.g., prophylactic) and palliative or ameliorative treatment. For such therapeutic uses, metalloprotease polypeptides such as TACE polypeptides and fragments, TACE nucleic acids encoding TACE polypeptides, and/or agonists or antagonists of the TACE polypeptide such as antibodies can be administered to the patient in need through well-known means. Compositions of the present invention can contain a polypeptide in any form described herein, such as native polypeptides, variants, derivatives, oligomers, and biologically active fragments.

Therapeutically Effective Amount. In practicing the method of treatment or use of the present invention, a therapeutically effective amount of a therapeutic agent of the present invention is administered to a patient having a condition to be treated. “Therapeutic agent” includes without limitation any of the TACE polypeptides, fragments, and variants; nucleic acids encoding the TACE family polypeptides, fragments, and variants; agonists or antagonists of the TACE polypeptides such as antibodies; TACE polypeptide binding partners; complexes formed from the TACE polypeptides, fragments, variants, and binding partners, etc. As used herein, the term “therapeutically effective amount” means the total amount of each therapeutic agent or other active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual therapeutic agent or active ingredient, administered alone, the term refers to that ingredient alone. When applied to a combination, the term refers to combined amounts of the ingredients that result in the therapeutic effect, whether administered in combination, serially or simultaneously. As used herein, the phrase “administering a therapeutically effective amount” of a therapeutic agent means that the patient is treated with said therapeutic agent in an amount and for a time sufficient to induce an improvement, and preferably a sustained improvement, in at least one indicator that reflects the severity of the disorder. An improvement is considered “sustained” if the patient exhibits the improvement on at least two occasions separated by one or more days, or more preferably, by one or more weeks. The degree of improvement is determined based on signs or symptoms, and determinations can also employ questionnaires that are administered to the patient, such as quality-of-life questionnaires. Various indicators that reflect the extent of the patient's illness can be assessed for determining whether the amount and time of the treatment is sufficient. The baseline value for the chosen indicator or indicators is established by examination of the patient prior to administration of the first dose of the therapeutic agent. Preferably, the baseline examination is done within about 60 days of administering the first dose. If the therapeutic agent is being administered to treat acute symptoms, the first dose is administered as soon as practically possible after the injury has occurred. Improvement is induced by administering therapeutic agents such as TACE polypeptides or antagonists until the patient manifests an improvement over baseline for the chosen indicator or indicators. In treating chronic conditions, this degree of improvement is obtained by repeatedly administering this medicament over a period of at least a month or more, e.g., for one, two, or three months or longer, or indefinitely. A period of one to six weeks, or even a single dose, often is sufficient for treating injuries or other acute conditions. Although the extent of the patient's illness after treatment may appear improved according to one or more indicators, treatment may be continued indefinitely at the same level or at a reduced dose or frequency. Once treatment has been reduced or discontinued, it later may be resumed at the original level if symptoms should reappear.

Dosing. One skilled in the pertinent art will recognize that suitable dosages will vary, depending upon such factors as the nature and severity of the disorder to be treated, the patient's body weight, age, general condition, and prior illnesses and/or treatments, and the route of administration. Preliminary doses can be determined according to animal tests, and the scaling of dosages for human administration is performed according to art-accepted practices such as standard dosing trials. For example, the therapeutically effective dose can be estimated initially from cell culture assays. The dosage will depend on the specific activity of the compound and can be readily determined by routine experimentation. A dose can be formulated in animal models to achieve a circulating plasma concentration range that includes the IC50 (i.e., the concentration of the test compound which achieves a half-maximal inhibition of symptoms) as determined in cell culture, while minimizing toxicities. Such information can be used to more accurately determine useful doses in humans. Ultimately, the attending physician will decide the amount of polypeptide of the present invention with which to treat each individual patient. Initially, the attending physician will administer low doses of polypeptide of the present invention and observe the patient's response. Larger doses of polypeptide of the present invention can be administered until the optimal therapeutic effect is obtained for the patient, and at that point the dosage is not increased further. It is contemplated that the various pharmaceutical compositions used to practice the method of the present invention should contain about 0.01 ng to about 100 mg (preferably about 0.1 ng to about 10 mg, more preferably about 0.1 microgram to about 1 mg) of polypeptide of the present invention per kg body weight. In one embodiment of the invention, TACE polypeptides or antagonists are administered one time per week to treat the various medical disorders disclosed herein, in another embodiment is administered at least two times per week, and in another embodiment is administered at least three times per week. If injected, the effective amount of TACE polypeptides or antagonists per adult dose ranges from 1-20 mg/m², and preferably is about 5-12 mg/m². Alternatively, a flat dose can be administered, whose amount may range from 5-100 mg/dose. Exemplary dose ranges for a flat dose to be administered by subcutaneous injection are 5-25 mg/dose, 25-50 mg/dose and 50-100 mg/dose. In one embodiment of the invention, the various indications described below are treated by administering a preparation acceptable for injection containing TACE polypeptides or antagonists at 25 mg/dose, or alternatively, containing 50 mg per dose. The 25 mg or 50 mg dose can be administered repeatedly, particularly for chronic conditions. If a route of administration other than injection is used, the dose is appropriately adjusted in accord with standard medical practices. In many instances, an improvement in a patient's condition will be obtained by injecting a dose of about 25 mg of TACE polypeptides or antagonists one to three times per week over a period of at least three weeks, or a dose of 50 mg of TACE polypeptides or antagonists one or two times per week for at least three weeks, though treatment for longer periods may be necessary to induce the desired degree of improvement. For incurable chronic conditions, the regimen can be continued indefinitely, with adjustments being made to dose and frequency if such are deemed necessary by the patient's physician. The foregoing doses are examples for an adult patient who is a person who is 18 years of age or older. For pediatric patients (age 4-17), a suitable regimen involves the subcutaneous injection of 0.4 mg/kg, up to a maximum dose of 25 mg of TACE polypeptides or antagonists, administered by subcutaneous injection one or more times per week. If an antibody against a TACE polypeptide is used as the TACE polypeptide antagonist, a preferred dose range is 0.1 to 20 mg/kg, and more preferably is 1-10 mg/kg. Another preferred dose range for an anti-TACE polypeptide antibody is 0.75 to 7.5 mg/kg of body weight. Humanized antibodies are preferred, that is, antibodies in which only the antigen-binding portion of the antibody molecule is derived from a non-human source. Such antibodies can be injected or administered intravenously.

Formulations. Compositions comprising an effective amount of a TACE polypeptide of the present invention (from whatever source derived, including without limitation from recombinant and non-recombinant sources), in combination with other components such as a physiologically acceptable diluent, carrier, or excipient, are provided herein. The term “pharmaceutically acceptable” means a non-toxic material that does not interfere with the effectiveness of the biological activity of the active ingredient(s). Formulations suitable for administration include aqueous and non-aqueous sterile injection solutions which can contain anti-oxidants, buffers, bacteriostats and solutes which render the formulation isotonic with the blood of the recipient; and aqueous and non-aqueous sterile suspensions which can include suspending agents or thickening agents. The polypeptides can be formulated according to known methods used to prepare pharmaceutically useful compositions. They can be combined in admixture, either as the sole active material or with other known active materials suitable for a given indication, with pharmaceutically acceptable diluents (e.g., saline, Tris-HCl, acetate, and phosphate buffered solutions), preservatives (e.g., thimerosal, benzyl alcohol, parabens), emulsifiers, solubilizers, adjuvants and/or carriers. Suitable formulations for pharmaceutical compositions include those described in Remington's Pharmaceutical Sciences, 16th ed. 1980, Mack Publishing Company, Easton, Pa. In addition, such compositions can be complexed with polyethylene glycol (PEG), metal ions, or incorporated into polymeric compounds such as polyacetic acid, polyglycolic acid, hydrogels, dextran, etc., or incorporated into liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323. Such compositions will influence the physical state, solubility, stability, rate of in vivo release, and rate of in vivo clearance, and are thus chosen according to the intended application, so that the characteristics of the carrier will depend on the selected route of administration. In one preferred embodiment of the invention, sustained-release forms of TACE polypeptides are used. Sustained-release forms suitable for use in the disclosed methods include, but are not limited to, TACE polypeptides that are encapsulated in a slowly-dissolving biocompatible polymer (such as the alginate microparticles described in U.S. Pat. No. 6,036,978), admixed with such a polymer (including topically applied hydrogels), and or encased in a biocompatible semi-permeable implant.

Combinations of Therapeutic Compounds. A TACE polypeptide of the present invention may be active in multimers (e.g., heterodimers or homodimers) or complexes with itself or other polypeptides. As a result, pharmaceutical compositions of the invention may comprise a polypeptide of the invention in such multimeric or complexed form. The pharmaceutical composition of the invention may be in the form of a complex of the polypeptide(s) of present invention along with polypeptide or peptide antigens. The invention further includes the administration of TACE polypeptides or antagonists concurrently with one or more other drugs that are administered to the same patient in combination with the TACE polypeptides or antagonists, each drug being administered according to a regimen suitable for that medicament. “Concurrent administration” encompasses simultaneous or sequential treatment with the components of the combination, as well as regimens in which the drugs are alternated, or wherein one component is administered long-term and the other(s) are administered intermittently. Components can be administered in the same or in separate compositions, and by the same or different routes of administration. Examples of components that can be administered concurrently with the pharmaceutical compositions of the invention are: cytokines, lymphokines, or other hematopoietic factors such as M-CSF, GM-CSF, TNF, IL-1, IL-2, IL-3, IL4, IL-5, IL-6, IL-7, IL-8, IL-9, IL-10, IL-11, IL-12, IL-13, IL-14, IL-15, IL-17, IL-18, IFN, TNF0, TNF1, TNF2, G-CSF, Meg-CSF, thrombopoietin, stem cell factor, and erythropoietin, or inhibitors or antagonists of any of these factors. The pharmaceutical composition can further contain other agents which either enhance the activity of the polypeptide or compliment its activity or use in treatment. Such additional factors and/or agents may be included in the pharmaceutical composition to produce a synergistic effect with polypeptide of the invention, or to minimize side effects. Conversely, a TACE polypeptide or antagonist of the present invention may be included in formulations of the particular cytokine, lymphokine, other hematopoietic factor, thrombolytic or anti-thrombotic factor, or anti-inflammatory agent to minimize side effects of the cytokine, lymphokine, other hematopoietic factor, thrombolytic or anti-thrombotic factor, or anti-inflammatory agent. Additional examples of drugs to be administered concurrently include but are not limited to antivirals, antibiotics, analgesics, corticosteroids, antagonists of inflammatory cytokines, non-steroidal anti-inflammatories, pentoxifylline, thalidomide, and disease-modifying antirheumatic drugs (DMARDs) such as azathioprine, cyclophosphamide, cyclosporine, hydroxychloroquine sulfate, methotrexate, leflunomide, minocycline, penicillamine, sulfasalazine and gold compounds such as oral gold, gold sodium thiomalate, and aurothioglucose.

Routes of Administration. Any efficacious route of administration can be used to therapeutically administer TACE polypeptides or antagonists thereof, including those compositions comprising nucleic acids. Parenteral administration includes injection, for example, via intra-articular, intravenous, intramuscular, intralesional, intraperitoneal or subcutaneous routes by bolus injection or by continuous infusion., and also includes localized administration, e.g., at a site of disease or injury. Other suitable means of administration include sustained release from implants; aerosol inhalation and/or insufflation.; eyedrops; vaginal or rectal suppositories; buccal preparations; oral preparations, including pills, syrups, lozenges, ice creams, or chewing gum; and topical preparations such as lotions, gels, sprays, ointments or other suitable techniques. Alternatively, polypeptideaceous TACE polypeptides or antagonists may be administered by implanting cultured cells that express the polypeptide, for example, by implanting cells that express TACE polypeptides or antagonists. Cells may also be cultured ex vivo in the presence of polypeptides of the present invention in order to modulate cell proliferation or to produce a desired effect on or activity in such cells. Treated cells can then be introduced in vivo for therapeutic purposes. The polypeptide of the instant invention may also be administered by the method of protein transduction. In this method, the TACE polypeptide is covalently linked to a protein-transduction domain (PTD) such as, but not limited to, TAT, Antp, or VP22 (Schwarze et al., 2000, Cell Biology 10: 290-295). The PTD-linked peptides can then be transduced into cells by adding the peptides to tissue-culture media containing the cells (Schwarze et al., 1999, Science 285: 1569; Lindgren et al., 2000, TiPS 21: 99; Derossi et al., 1998, Cell Biology 8: 84; WO 00/34308; WO 99/29721; and WO 99/10376). In another embodiment, the patient's own cells are induced to produce TACE polypeptides or antagonists by transfection in vivo or ex vivo with a DNA that encodes TACE polypeptides or antagonists. This DNA can be introduced into the patient's cells, for example, by injecting naked DNA or liposome-encapsulated DNA that encodes TACE polypeptides or antagonists, or by other means of transfection. Nucleic acids of the invention can also be administered to patients by other known methods for introduction of nucleic acid into a cell or organism (including, without limitation, in the form of viral vectors or naked DNA). When TACE polypeptides or antagonists are administered in combination with one or more other biologically active compounds, these can be administered by the same or by different routes, and can be administered simultaneously, separately or sequentially.

Oral Administration. When a therapeutically effective amount of polypeptide of the present invention is administered orally, polypeptide of the present invention will be in the form of a tablet, capsule, powder, solution or elixir. When administered in tablet form, the pharmaceutical composition of the invention can additionally contain a solid carrier such as a gelatin or an adjuvant. The tablet, capsule, and powder contain from about 5 to 95% polypeptide of the present invention, and preferably from about 25 to 90% polypeptide of the present invention. When administered in liquid form, a liquid carrier such as water, petroleum, oils of animal or plant origin such as peanut oil, mineral oil, soybean oil, or sesame oil, or synthetic oils can be added. The liquid form of the pharmaceutical composition can further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of polypeptide of the present invention, and preferably from about 1 to 50% polypeptide of the present invention.

Intravenous Administration. When a therapeutically effective amount of polypeptide of the present invention is administered by intravenous, cutaneous or subcutaneous injection, polypeptide of the present invention will be in the form of a pyrogen-free, parenterally acceptable aqueous solution. The preparation of such parenterally acceptable polypeptide solutions, having due regard to pH, isotonicity, stability, and the like, is within the skill in the art. A preferred pharmaceutical composition for intravenous, cutaneous, or subcutaneous injection should contain, in addition to polypeptide of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention can also contain stabilizers, preservatives, buffers, antioxidants, or other additives known to those of skill in the art. The duration of intravenous therapy using the pharmaceutical composition of the present invention will vary, depending on the severity of the disease being treated and the condition and potential idiosyncratic response of each individual patient. It is contemplated that the duration of each application of the polypeptide of the present invention will be in the range of 12 to 24 hours of continuous intravenous administration. Ultimately the attending physician will decide on the appropriate duration of intravenous therapy using the pharmaceutical composition of the present invention.

Bone and Tissue Administration. For compositions of the present invention which are useful for bone, cartilage, tendon or ligament disorders, the therapeutic method includes administering the composition topically, systematically, or locally as an implant or device. When administered, the therapeutic composition for use in this invention is, of course, in a pyrogen-free, physiologically acceptable form. Further, the composition can desirably be encapsulated or injected in a viscous form for delivery to the site of bone, cartilage or tissue damage. Topical administration may be suitable for wound healing and tissue repair. Therapeutically useful agents other than a polypeptide of the invention which may also optionally be included in the composition as described above, can alternatively or additionally, be administered simultaneously or sequentially with the composition in the methods of the invention. Preferably for bone and/or cartilage formation, the composition would include a matrix capable of delivering the polypeptide-containing composition to the site of bone and/or cartilage damage, providing a structure for the developing bone and cartilage and optimally capable of being resorbed into the body. Such matrices can be formed of materials presently in use for other implanted medical applications. The choice of matrix material is based on biocompatibility, biodegradability, mechanical properties, cosmetic appearance and interface properties. The particular application of the compositions will define the appropriate formulation. Potential matrices for the compositions can be biodegradable and chemically defined calcium sulfate, tricalciumphosphate, hydroxyapatite, polylactic acid, polyglycolic acid and polyanhydrides. Other potential materials are biodegradable and biologically well-defined, such as bone or dermal collagen. Further matrices are comprised of pure polypeptides or extracellular matrix components. Other potential matrices are nonbiodegradable and chemically defined, such as sintered hydroxapatite, bioglass, aluminates, or other ceramics Matrices can be comprised of combinations of any of the above mentioned types of material, such as polylactic acid and hydroxyapatite or collagen and tricalciumphosphate. The bioceramics can be altered in composition, such as in calcium-aluminate-phosphate and processing to alter pore size, particle size, particle shape, and biodegradability. Presently preferred is a 50:50 (mole weight) copolymer of lactic acid and glycolic acid in the form of porous particles having diameters ranging from 150 to 800 microns. In some applications, it will be useful to utilize a sequestering agent, such as carboxymethyl cellulose or autologous blood clot, to prevent the polypeptide compositions from disassociating from the matrix. A preferred family of sequestering agents is cellulosic materials such as alkylcelluloses (including hydroxyalkylcelluloses), including methylcellulose, ethylcellulose, hydroxyethylcellulose, hydroxypropylcellulose, hydroxypropyl-methylcellulose, and carboxymethyl-cellulose, the most preferred being cationic salts of carboxymethylcellulose (CMC). Other preferred sequestering agents include hyaluronic acid, sodium alginate, poly(ethylene glycol), polyoxyethylene oxide, carboxyvinyl polymer and poly(vinyl alcohol). The amount of sequestering agent useful herein is 0.5-20 wt %, preferably 1-10 wt % based on total formulation weight, which represents the amount necessary to prevent desorbtion of the polypeptide from the polymer matrix and to provide appropriate handling of the composition, yet not so much that the progenitor cells are prevented from infiltrating the matrix, thereby providing the polypeptide the opportunity to assist the osteogenic activity of the progenitor cells. In further compositions, polypeptides of the invention may be combined with other agents beneficial to the treatment of the bone and/or cartilage defect, wound, or tissue in question. These agents include various growth factors such as epidermal growth factor (EGF), platelet derived growth factor (PDGF), transforming growth factors (TGF-alpha and TGF-beta), and insulin-like growth factor (IGF). The therapeutic compositions are also presently valuable for veterinary applications. Particularly domestic animals and thoroughbred horses, in addition to humans, are desired patients for such treatment with polypeptides of the present invention. The dosage regimen of a polypeptide-containing pharmaceutical composition to be used in tissue regeneration will be determined by the attending physician considering various factors which modify the action of the polypeptides, e.g., amount of tissue weight desired to be formed, the site of damage, the condition of the damaged tissue, the size of a wound, type of damaged tissue (e.g., bone), the patient's age, sex, and diet, the severity of any infection, time of administration and other clinical factors. The dosage can vary with the type of matrix used in the reconstitution and with inclusion of other polypeptides in the pharmaceutical composition. For example, the addition of other known growth factors, such as IGF I (insulin like growth factor I), to the final composition, may also effect the dosage. Progress can be monitored by periodic assessment of tissue/bone growth and/or repair, for example, X-rays, histomorphometric determinations and tetracycline labeling.

Veterinary Uses. In addition to human patients, TACE polypeptides and antagonists are useful in the treatment of disease conditions in non-human animals, such as pets (dogs, cats, birds, primates, etc.), domestic farm animals (horses cattle, sheep, pigs, birds, etc.), or any animal that suffers from a TACE-mediated condition. In such instances, an appropriate dose can be determined according to the animal's body weight. For example, a dose of 0.2-1 mg/kg may be used. Alternatively, the dose is determined according to the animal's surface area, an exemplary dose ranging from 0.1-20 mg/m², or more preferably, from 5-12 mg/m². For small animals, such as dogs or cats, a suitable dose is 0.4 mg/kg. In a preferred embodiment, TACE polypeptides or antagonists (preferably constructed from genes derived from the same species as the patient), is administered by injection or other suitable route one or more times per week until the animal's condition is improved, or it can be administered indefinitely.

Manufacture of Medicaments. The present invention also relates to the use of TACE polypeptides, fragments, and variants; nucleic acids encoding the TACE family polypeptides, fragments, and variants; agonists or antagonists of the TACE polypeptides such as antibodies; TACE polypeptide binding partners; complexes formed from the TACE family polypeptides, fragments, variants, and binding partners, etc, in the manufacture of a medicament for the prevention or therapeutic treatment of each medical disorder disclosed herein.

### EXAMPLES

The following examples are intended to illustrate particular embodiments and not to limit the scope of the invention.

### Example 1

### Identification of Metalloprotease-Shed Proteins in Monocytes

Many metalloprotease-mediated shedding events are induced by phorbol esters such as phorbol 12-myristate 13-acetate (PMA), and metalloproteases are inhibited for example by hydroxamic acid-compounds such as IC3 (Hooper et al., 1997, Biochem J 321: 265-279; Mohler et al., 1994, Nature 370: 218-220). In order to isolate shed proteins, cell supernatants were collected from wild-type mouse bone marrow-derived monocytic (DRM) cells (Peschon et al., 1998, Science 282: 1281-1284) that were cultured as described by Rovida et al., 2001, J Immunol 166: 1583-1589, and stimulated with PMA in the presence or absence of IC3, as follows. Prior to stimulation, DRM cells were expanded in one-liter spinner flasks, seeded at 2.5×10⁵ cells/ml and grown to approximately 2×10⁶-3×10⁶ cells/ml in 800 ml growth media. DRM cells were prepared for stimulation by washing twice with cold, serum-free RPMI 1640 (Life Technologies, Rockville, Md.), and once in cold, phenol red free, serum-free RPMI 1640 (Life Technologies). Washed cells were placed in T175 flasks at 8×10⁶ cells/ml in 25 ml phenol red and serum free RPMI 1640. IC3 (25 micrograms/ml) and/or PMA (100 ng/ml) (ICN Biomedicals, Inc., Aurora, Ohio) were added to appropriate flasks. Flasks were incubated 90 minutes at 37 degrees C. with 5% CO₂. Supernatants from all flasks were harvested, centrifuged 10 minutes, 1200 rpm, 4 degrees C.; 0.22 micrometer filtered (Corning Inc., Corning, N.Y.) and treated with protease inhibitors (175 micrograms/ml PMSF, 4.75 micrograms/ml Leupeptin, 6.9 micrograms/ml Pepstatin A and 2.5 micrograms/ml EDTA). Supernatants were concentrated (Centricon Plus-80, 10 Kd cut-off, Millipore, Bedford, Mass.; for volumes up to 80 ml) prior to purification.

From six separate experiments, an average of 4.0 mg of supernatant proteins were derived from 10⁹ cells in the presence of IC3; from nine separate experiments, an average 4.3 mg per 10⁹ cells was obtained in the absence of IC3. Since no statistically significant differences were detected in the total amount of protein in the two samples, it was deduced that metalloprotease-shed proteins composed a small fraction of the total, and that the majority of the supernatant proteins were derived from normal cell turnover and metabolism. This was confirmed when the supernatant proteins were digested with trypsin, and analyzed by tandem mass spectrometry (MS/MS). These data showed that the most prominent proteins in the cell supernatant were various forms of heat shock proteins, actin and metabolic pathway enzymes. Consistent with this observation, we were unable to discern any differences in the staining pattern on two-dimensional (2D) (isoelectric focusing and sodium dodecyl sulfate (SDS)) polyacrylamide gel electrophoresis (PAGE) gels obtained from pairs of cell supernatants (with and without IC3) (Panel A of FIG. 1 and data not shown). The first dimension of the 2-D separation was carried out using immobilized 11-cm IPG strips from BioRad (Hercules, Calif.). The deglycosylated proteins were mixed with rehydration buffer (8M urea, 2% CHAPS, 45 mM DTT, 0.5% ampholytes pH 3-10 (BioRad), and 0.0002% bromphenol blue. Isoelectric focusing was performed using the IPGphor system from Amersham Pharmacia Biotech Inc. (Piscataway, N.J.). The 4-20% gradient Criterion gels from BioRad were used for the second dimension. Protein bands/spots were detected by staining with Colloidal Blue (Invitrogen).

Although 2D-PAGE is widely used and is recognized as a basic tool for proteomics, it seems to display only the most abundant proteins in a complex sample (Gygi et al., 2000, Proc Natl Acad Sci USA 97: 9390-9395; and Smith, 2000, Nat Biotechnol 18: 1041-1042). Hence, it was evident that additional protein fractionation would be required in order to discern quantitative differences between lower abundance proteins in these samples. Because most cell-surface proteins contain one or more carbohydrate groups, proteins released from cell membranes are likely to be glycosylated. Wheat germ agglutinin (WGA), which contains a group of closely related isolectins, can bind oligosaccharides containing sialic acid or terminal N-acetylglucosamine that are common to many mammalian secreted and membrane glycoproteins. Therefore, agarose-bound wheat germ agglutinin (WGA) (Vector Laboratories, Inc., Burlingame, Calif.) was chosen for the affinity purification of glycoproteins from the cell supernatants. Briefly, two to four mg of concentrated supernatant proteins were incubated with 250 microliters of washed WGA agarose beads in 4 ml of 10 mM HEPES, pH7.5 containing 0.15 M NaCl (HEPES/NaCl buffer) in a capped micro Bio-spin chromatography column (BioRad, Hercules, Calif.). After incubating at 4 degrees C. for 1 hour on a rotary shaker, the column was washed three times with 5 ml of the HEPES/NaCl buffer. The lectin-binding proteins were then eluted with 3 ml of 0.5 M N-acetyl-D-glucosamine in HEPES/NaCl buffer. The excess amount of N-acetyl-D-glucosamine was removed from the WGA eluate by 7.5 fold concentration (Centricon®, YM-10, 10 Kd cut-off, Millipore, Bedford, Mass., for volumes up to 2 ml), followed by protein precipitation at room temperature using a method designed for quantitative recovery of protein in dilute solution in the presence of detergents and lipids (Wessel and Flugge, 1984, Anal Biochem 138: 141-143). After the lectin affinity fractionation, the isolated glycoproteins were subjected to N-deglycosylation by treatment with recombinant N-glycosidase F, also referred to as N-glycanase or PNGaseF (Glyko, Inc., Novato, Calif.), according to the vendor's instructions. This treatment had the effect of reducing glycoprotein heterogeneity, and therefore enhancing the protein focusing on sodium dodecyl sulfate polyacrylamide gel electrophoresis (SDS PAGE) gels.

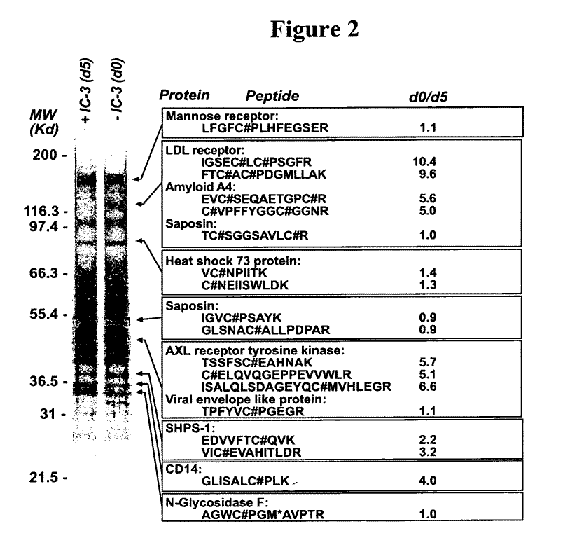

The N-deglycosylated proteins were analyzed by both 2D- and 1D-PAGE (Panel B of FIG. 1 and FIG. 2). 2D-PAGE was performed as described above; 1D-PAGE was performed under reducing conditions using Tris-glycine 4-20% gradient gels (Novex gel, Invitrogen, Carlsbad, Calif.). When compared to the samples from cultures containing IC3 (data not shown), a few 2D-PAGE spots were determined to be unique or of increased intensity in the supernatants obtained from cells not treated with IC3 (cells were stimulated with PMA in both cases) (Panel B of FIG. 1). These spots were not detectable prior to WGA-enrichment of glycoproteins (Panel A of FIG. 1), which apparently was due to their relatively low abundance in the unfractionated cell supernatant. Gel pieces containing these spots were excised, and their protein content was identified by tandem mass spectrometry after in-gel digestion with trypsin (Table 1). Except for saposin and tubulin, the proteins that were identified from the 2D-PAGE experiment are type 1 transmembrane proteins (Table 1), thus indicating that the lectin affinity step was reasonably effective in eliminating cytoplasmic proteins. All of the tryptic peptides identified (Table 1) were derived from the extracellular domains of the corresponding membrane proteins, as predicted for proteins released by shedding.

TABLE 1

Peptide sequences identified by tandem mass spectrometry following
in-gel trypsin digestion of 2D-PAGE spots (FIG. 1, Panel B).

| | | | SEQ |
|---|---|---|---|
| | | Peptide sequences identi- | ID |
| Protein | Protein Description^{a} | fied by MS/MS^{b} | NO |
| A4 = | Alzheimer's disease amyloid | CLVGEFVSDALLVPDK | 13 |
| Amyl A4 | A4 protein GenPept: P12023 | | |
| | | mDVCETHLHWHTVAK | 14 |
| | | CVPFFYGGCGGNR | 15 |
| | | STNLHDYGmLLPCGIDK | 16 |
| | | EQNYSDDVLANmISEPR | 17 |
| | | VESLEQEAANER | 18 |
| | | ISYGNDALmPSLTETK | 19 |
| | | WYFDVTEGK | 20 |
| | | mDAEFGHDSGFEVR | 21 |
| | | YLETPGDENEHAHFQK | 22 |
| AXLr | AXL receptor tyrosine kinase | CELQVQGEPPEVVWLR | 23 |
| | GenPept: NP_033491 | | |
| | | DTQTEAGSPFVGNPGD^{c}ITGAR | 24 |
| | | VPLQGTLLGYR | 25 |
| | | ISALQLSDAGEYQCMVHLEGR | 26 |
| | | GQDTPEVLmDIGLTR | 27 |
| | | EVTLELR | 28 |
| IL-1R-2 | Interleukin 1 receptor, type | EDLHTDFK | 29 |
| | II GenPept: NP_034685 | | |
| | | GTTTEPIPVIISPLETIPASLGSR | 30 |
| | | EFLSAGDPTR | 31 |
| | | GNILWILPAVQQDSGTYICTFR | 32 |
| | | LDSSQLIPR | 33 |
| | | LEGEPVVLR | 34 |
| | | GNKEFLSAGDPTR | 35 |
| | | VKGTTTEPIPVIISPLETIPASLGSR | 36 |
| | | D^{c}ASHCEQmSVELK | 37 |
| | | LLISD^{c}TSmDDAGYYR | 38 |
| IL-6R-1 | Interleukin 6 receptor, alpha | EWTTTGNTLVLR | 39 |
| | GenPept: P22272 | | |
| | | SDFQVPCQYSQQLK | 40 |
| | | ALEVAD^{c}GTVTSLPGATVTLICPGK | 41 |
| LDLr | Low density lipoprotein re- | AVGSIGYLLFTNR | 42 |
| | ceptor GenPept: I48623 | | |
| | | LYWVDSK | 43 |
| | | CHSGECISLDK | 44 |
| | | NIYWTDSVPGSVSVADTK | 45 |
| | | IGSECLCPSGFR | 46 |
| | | NVVALDTEVTNNR | 47 |
| | | IYWSDLSQK | 48 |
| | | SEYTSLLPNLK | 49 |
| | | LAHPFSLAIYEDK | 50 |
| | | SWVCDGEADCK | 51 |
| | | TILEDENR | 52 |
| | | LTGSDVNLVAENLLSPEDIVLFHK | 53 |
| | | LHSISSIDVD^{c}GGNR | 54 |
| L-selectin | Selectin, lymphocyte | EIEYLENTLPK | 55 |
| | GenPept: NP_035476 | | |
| | | SKEDCVEIYIK | 56 |
| | | QNYTDLVAIQNK | 57 |
| | | SPYYYWIGIR | 58 |
| | | MWTWVGTD^{c}K | 59 |
| | | QD^{c}YTDLVAIQNK | 60 |
| | | AALCYTASCQPGSCD^{c}GR | 61 |
| c-FMS = | Fms proto-oncogene = | ASEAGQYFLmAQNK | 62 |
| M-CSFR | Macrophage colony stimulating | | |
| | factor 1 receptor GenPept: | | |
| | P09581 | | |
| | | VIIQSQLPIGTLK | 63 |
| | | KLEFITQR | 64 |
| | | VLDSNTYVCK | 65 |
| | | TVYFFSPWR | 66 |
| Met | Met proto-oncogene (hepato- | TGPVLEHPDCLPCR | 67 |
| | cyte growth factor receptor) | | |
| | GenGept: NP_032617 | | |
| | | YIHAFESNHFIYFLTVQK | 68 |
| | | ETLDAQTFHTR | 69 |
| | | DNINmALLVDTYYDDQLISCGSVNR | 70 |
| | | FCSVDSGLHSYmEmPLECILTEK | 71 |
| | | FINFFVGNTID^{c}SSYPPGYSLHSISVR | 72 |
| SHPS-1 | SHP substrate 1 protein | LLIYSFTGEHFPR | 73 |
| | GenPept: JC5289 | | |
| Saposin | Saposin precursor GenPept: | EVVDSYLPVILDmIK | 74 |
| | JH0604 | | |
| | | QLESNKIPEVDmAR | 75 |
| | | LVSDVQTAVK | 76 |
| | | VVAPFmSNIPLLLYPQDHPR | 77 |
| | | TD^{c}SSFIQGFVDHVK | 78 |
| Tubulin | Tubulin, beta 5 GenPept: | YLTVAAVFR | 79 |
| | NP_03578S | | |
| | | ImNTFSVVPSPK | 80 |
| | | LHFFmPGFAPLTSR | 81 |
| | | ALTVPELTQQVFDAK | 82 |
| | | GHYTEGAELVDSVLDVVR | 83 |

^{a}Protein descriptions were obtained from the Entrez website: ncbi.nlm.nih.gov:80/entrez
^{b}Lower case “m” indicates methionine sulfoxide.
^{c}N-glycosylation site: N is enzymatically converted to D due to the N-glycosidase F treatment.

Although N-deglycosylation reduces protein heterogeneity, it does not eliminate it. Hence, due to differences in isoelectric point and/or molecular weight shifts resulting from O-glycosylation and other modifications, most proteins appeared as multiple spots on 2D-PAGE gels, and many of the spots contained more than one protein (Panel B of FIG. 1). This makes protein quantitation via gel scanning and densitometry quite difficult. To overcome this problem, we established a protein quantitation method that combines 1D-PAGE with stable isotope dilution. Proteins are first fractionated by 1D-PAGE (FIG. 2). Matching pairs of protein bands with the same molecular weight (with and without IC3) were then excised from the 1D gel, and destained by washing with a mixture of 200 mM NH₄HCO₃/acetonitrile (1:1). Proteins were reduced with DTT, cysteines were alkylated with either isotopically light N-ethyl iodoacetamide (d0) or heavy N-d₅-ethyl-iodoacetamide (d5), and digested in-gel with trypsin trypsin (Promega, Madison, Wis.) as described (Shevchenko et al., 1996, Anal Chem 68: 850-858). N-ethyl-iodoacetamide (either d0 or d5 form) was synthesized from ethylamine hydrochloride (either d0 or d5 form) and iodoacetic anhydride. The tryptic digests were combined, concentrated by vacuum centrifugation, and analyzed by mass spectrometric analysis.

Mass spectrometric analysis of tryptic peptides was performed on a Micromass QTOF 1 instrument (Microssmass UK Ltd, Wythenshawe, Manchester, United Kingdom). Peptides were sequenced by on-line microcapillary liquid chromotograhy-electrospray ionization-tandem mass spectrometry (MS/MS) analysis using a LCpackings (San Francisco, Calif.) 50 micron 1D C₁₈ column. The gradient was developed using an Eldex Micropro pump (Napa, Calif.) operating at 5 microliters/min, and the flow was split before the injector such that the flow rate through the column was approximately 250 nl/min. The effluent of the column was directed into an Upchurch (Oak Harbor, Wash.) micro-tee containing a platinum electrode and a New Objective (Cambridge, Mass.) uncoated fused silica tip (360 micron OD, 20 micron ID, pulled to a 10 micron opening). The mass spectrometer was operated in a data-dependent MS/MS mode and proteins were identified by searching a non-redundant protein sequence database using the Mascot program (Perkins et al., 1999, Electrophoresis 20: 3551-3567). A second LC/MS acquisition (MS-only mode) was performed for each sample in order to generate accurate ion intensity data for quantitation.

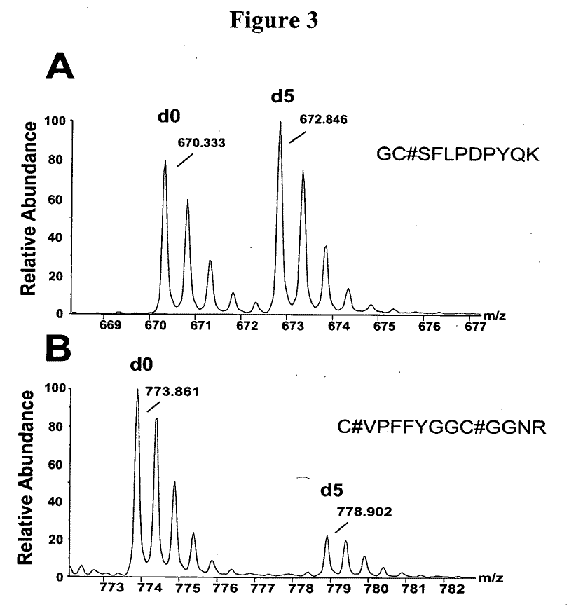

Proteins that were identified from the 1D-PAGE gel included all the proteins that were identified in the 2D-gel experiments (Panel B of FIG. 1, Table 1). In addition, for those proteins from which data could be obtained for cysteine-containing peptides, relative quantitation was determined by comparing the intensity of the d0 and d5 ions (FIG. 2). Two examples of these ion pairs used for quantitation are shown (FIG. 3). Comparison of the d0 versus d5 intensity revealed ratios close to 1 for peptides obtained from saposin, heat shock 73 protein, and N-glycosidase F (FIG. 2). A ratio of 1 was expected for the N-glycosidase F because an equal amount of N-glycosidase F was added to each sample during the deglycosylation treatment. Saposin and heat shock 73 protein were among the most abundant proteins in the cell supernatant before lectin purification and represent non-metalloprotease mediated shed and secreted proteins, respectively. In contrast, several membrane proteins, including LDLr, amyloid A4 protein, AXLr, SHPS-1, and CD14, were determined to be in greater abundance in the sample lacking IC3 (FIG. 2). We conclude that these proteins were shed via a metalloprotease that can be inhibited by IC3.

This experiment was repeated several times, and the 1D-PAGE gel patterns were very reproducible with the exception of a very high molecular weight protein (>200 kDa) named hybrid receptor SorLA (GenPept: O88307). In most cases, the staining pattern indicated that SorLA was shed in the absence of IC3, but not in the presence of the metalloprotease inhibitor. In a few cases (FIG. 2), the shedding of SorLA was not apparent. The reason for the absence of SorLA in this particular gel is unknown, but it may be due variability in gel quality or that this large protein may not migrate reproducibly.

### Example 2

### Identification of TACE-Mediated Shedding in Monocytes

To link the above shedding events specifically with TACE activity, TACE−/− DRM cells (Peschon et al., 1998, Science 282: 1281-1284) were reconstituted with full-length TACE. A TACE-encoding retrovirus was generated as described (Kinsella and Nolan, 1996, Hum Gene Therapy 7: 1405-1413), and used to reconstitute functional full-length TACE in TACE−/− DRM cells. The control cells were generated by transfecting TACE−/− DRM cells with retrovirus containing an empty vector. The expression of TACE was confirmed by a functional reconstitution assay in which DRM TACE−/− monocytes were stimulated with LPS (1 microgram/ml), and shedding of TNF and TNFR were analyzed by ELISA (Pharmingen, OptEIA™, San Diego, Calif.). Comparison of the protein shedding profiles of the TACE-reconstituted cell line with that obtained from TACE−/− cells transfected with an empty vector revealed visible differences by 1D-PAGE (FIG. 4). Quantitative analysis of selected areas cut from the 1D-PAGE gel showed changes in peptide quantities for several proteins, including hybrid receptor SorLA, LDLr, Amyloid A4, AXLr, IL-1R-2 and IL-6R-1. These proteins are therefore most likely shed by TACE.

### Example 3

### Identification of Metalloprotease-Shed Proteins in Endothelial Cells

To determine whether this approach can be used to identify proteins shed by other cell types, we carried out a study with human adult dermal microvascular endothelial cells (HMVECs). HMVECs (BioWhittaker/Clonetics, Walkersville, Md.) were grown in EGM2MV media (BioWhittaker/Clonetics, Walkersville, Md.) to passage 6. Cultures were fed with fresh media every 2-3 days, and passed every 5 days. To pass, 80-90% confluent cultures were gently trypsinized (BioWhittaker/Clonetics, Walkersville, Md.) and T175 flasks were seeded at 10,000 cells/cm² in 35 ml media.

HMVECs were treated with a mixture of inflammatory cytokines followed by PMA to induce shedding, as follows. Passage 6, 90% confluent cells were used. Growth medium was gently replaced with EBM-2 basal media (BioWhittaker/Clonetics, Walkersville, Md.) and cultures were incubated for 14 hours. Medium was gently replaced again with phenol red-free EBM basal media (BioWhittaker/Clonetics, Walkersville, Md.) and half the flasks were supplemented with an inflammatory cytokine cocktail for 4 hours. The cytokine cocktail is composed of 100 ng/ml human CD40 ligand (hCD40L, Immunex, Seattle, Wash.); 2 ng/ml hIL-1-beta (Immunex, Seattle, Wash.); 2 ng/ml hTNF-alpha (BioSource International, Inc., Camarillo, Calif.); 100 U/ml hIFN-gamma (BioSource International, Inc., Camarillo, Calif.); 30 ng/ml hFGF-basic (Chemicon International, Inc., Temecula, Calif.); 100 ng/ml hTWEAK (Chemicon International., Temecula, Calif.) and 10 ng/ml hVEGF (Chemicon International., Temecula, Calif.). After 4 hours, PMA (100 ng/ml) (ICN Biomedicals) was added to the cytokine-containing flasks, which were incubated for an additional hour. Supernatants from all flasks were harvested as above. For cytokine-stimulated cells the total supernatant protein yield per 10⁸ cells was 6.3 mg; whereas, unstimulated control cells yielded 3.0 mg.

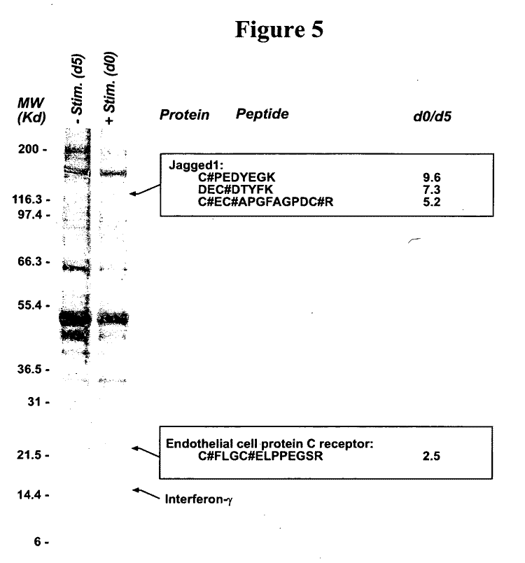

After lectin affinity purification and N-deglycosylation, the supernatant proteins from the HMVECs were analyzed by 1D-PAGE (FIG. 5). Overall, the two protein profiles were very similar and some of the discrepancies could be attributed to the cytokines added as part of the cell stimulation (e.g., the band labeled as interferon-gamma). However, two HMVEC-derived proteins, Jagged1 and endothelial cell protein C receptor, were identified from protein bands which appear to be of greater staining intensity in the cytokine/PMA treated sample (FIG. 5). Protein quantification using the isotope-coded differential cysteine labeling method demonstrated that these two proteins were indeed more abundant in the stimulated cell supernatant (FIG. 5). Although we did not determine the effect of IC3 on their release, both are transmembrane proteins and thus likely to be released by shedding. In fact, endothelial cell protein C receptor was previously identified as a metalloprotease-shed protein in endothelial cells (Xu et al., 2000, J Biol Chem 275: 6038-6044), thus validating the method as applied to HMVECs.

### Example 4

### Additional Experiments to Identify Metalloprotease-Shed Proteins in Monocytes

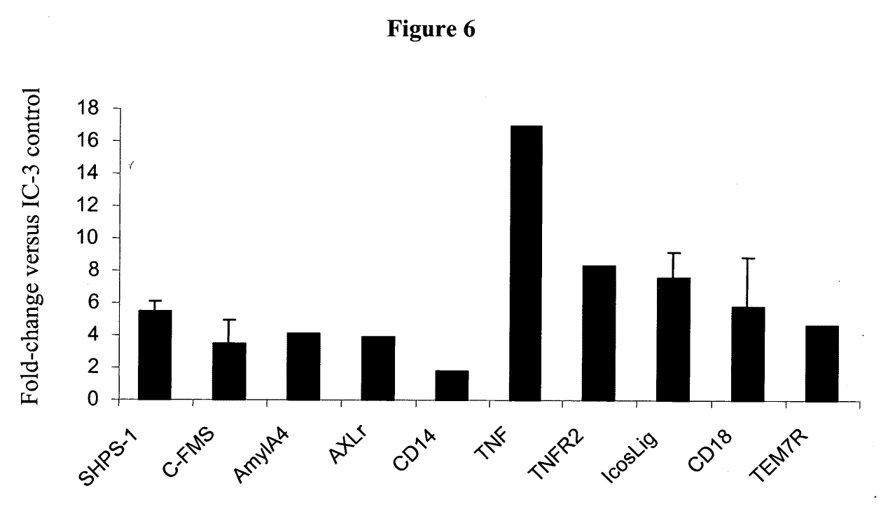

Cell culture, stimulation, lectin-affinity purification, and preparation of protein mixtures. Murine Dexter-ras-myc (DRM) monocytic cells were cultured as described in Example 1 above. Cell stimulation was performed in the same manner as in Example 1, except that 1 microgram/ml lipopolysaccharide (LPS) was also added 4 hours prior to the addition of phorbol 12-myristate 13-acetate (PMA). As described in Example 1, glycoproteins were isolated using a wheat germ agglutinin (WGA) column, followed by protein precipitation to remove lipids and salts. The protein pellet was solubilized in 25 microliters 8 M urea and 1 microliter was used to measure the total protein content using a Micro BCA kit (Pierce Chemical Co., Rockford, Ill.). The amount of total protein for the lectin-purified glycoproteins was approximately 40 micrograms. A new method was used to determine the ratio of heavy to light isotope ion intensity. For most peptides this ratio was about 0.56, which presumably represents the ratio of total protein present in one sample over the other. In a few cases, the ratio of heavy to light isotope ion intensity was quite different (Table 2 below), and many of these peptides were identified as being derived from proteins that we identified in previous experiments as being inducibly shed. To obtain the relative change in protein quantities for the inducibly shed proteins (as shown graphically in FIG. 6), the ratios in Table 2 were normalized by the ratio (0.56) observed for the constitutively shed proteins. Five of these inducibly shed proteins—amyloid A4, AXL receptor, c-FMS (or M-CSFR), SHPS-1, and CD14—were also identified as inducibly shed in our previous experiments. Two of them—TNF and TNFR2—are known to be proteins shed by TACE (Black et al., 1997, Nature 385: 729-33; Peschon et al., 1998, Science 282: 1281-1284.) The remaining three proteins from FIG. 6—ICOS ligand, CD18, and tumor endothelial marker 7-related (TEM7R)—have not previously been identified as proteins subject to inducible shedding by metalloproteases. The identification of proteins previously known to be shed validates the method, and also provides confidence that the new proteins are also shed molecules.

TABLE 2

Ratio of ion intensity of heavy versus light
isotope labeled peptides. Most peptide ion
pairs had an ion intensity ratio of 0.56,
which represents the relative amounts of total
protein in each sample. The supernatant
proteins obtained from PMA and LPS stimulation
in the presence or absence of the metallo-
protease inhibitor IC3 were labeled with light
and heavy isotope reagents, respectively.

| | | | | |
|---|---|---|---|---|
| | | | | SEQ |
| | | | Normalized | ID |
| Protein | Peptide | Ratio | Ratio* | NO |

| | | | | |
|---|---|---|---|---|
| SHPS-1 | VICEVAHITLDR | 3.1 | 5.5 | 137 |
| | NNMDFSIR | 2.8 | 5.0 | 138 |
| | VVLNSMDVHSK | 3.1 | 5.5 | 139 |
| | LLIYSFTGEHFPR | 3.2 | 5.7 | 140 |
| c-FMS = | VLDSNTYVCK | 1.6 | 2.9 | 141 |
| M-CSFR | KLEFITQR | 2.4 | 4.3 | 142 |
| | VIIQSQLPIGTLK | 1.7 | 3.0 | 143 |
| | ASEAGQYFLMAQNK | 2.2 | 3.9 | 144 |
| Amyl A4 | SQVMTHLR | 2.3 | 4.1 | 145 |
| | QQLVETHMAR | 2.3 | 4.1 | 146 |
| AXLr | TSSFSCEAHNAK | 2.2 | 3.9 | 147 |
| CD14 | NAGMETPSGVCSALAAAR | 1.0 | 1.8 | 148 |
| TNF | GQGCPDYVLLTHTVSR | 9.4 | 16.8 | 149 |
| TNFR2 | VCACEAGR | 4.6 | 8.2 | 150 |
| ICOS | NVTPQDTQEFTCR | 2.7 | 4.8 | 151 |
| ligand | TYTCMSK | 4.4 | 7.9 | 152 |
| | LGLYDVISTLR | 5.3 | 9.5 | 153 |
| | VFMNTATELVK | 4.6 | 8.2 | 154 |
| CD18 | STTGCLNAR | 3.3 | 5.9 | 155 |
| | YNSQVCGGSDR | 4.0 | 7.1 | 156 |
| | SRGDCDGVQINNPVTFQVK | 2.3 | 4.1 | 157 |
| TEM7R | HRQDWVDSGCPEEVQSK | 2.5 | 4.5 | 158 |

*The normalized ratio is the ratio divided by 0.56

### Example 5

### Antisense Inhibition of TACE Expression

In accordance with the present invention, a series of oligonucleotides are designed to target different regions of mRNA molecules encoding TACE polypeptides as described in U.S. Pat. Nos. 5,830,742 and 6,013,466, which are incorporated by reference herein. The oligonucleotides are selected to be approximately 10, 12, 15, 18, or more preferably 20 nucleotide residues in length, and to have a predicted hybridization temperature that is at least 37 degrees C. Preferably, the oligonucleotides are selected so that some will hybridize toward the 5′ region of the mRNA molecule, others will hybridize to the coding region, and still others will hybridize to the 3′ region of the mRNA molecule. Methods such as those of Gray and Clark (U.S. Pat. Nos. 5,856,103 and 6,183,966) can be used to select oligonucleotides that form the most stable hybrid structures with target sequences, as such oligonucleotides are desirable for use as antisense inhibitors.

The oligonucleotides may be oligodeoxynucleotides, with phosphorothioate backbones (internucleoside linkages) throughout, or may have a variety of different types of internucleoside linkages. Generally, methods for the preparation, purification, and use of a variety of chemically modified oligonucleotides are described in U.S. Pat. No. 5,948,680. As specific examples, the following types of nucleoside phosphoramidites may be used in oligonucleotide synthesis: deoxy and 2′-alkoxy amidites; 2′-fluoro amidites such as 2′-fluorodeoxyadenosine amidites, 2′-fluorodeoxyguanosine, 2′-fluorouridine, and 2′-fluorodeoxycytidine; 2′-O-(2-methoxyethyl)-modified amidites such as 2,2′-anhydro[1-(beta-D-arabino-furanosyl)-5-methyluridine], 2′-O-methoxyethyl-5-methyluridine, 2′-O-methoxyethyl-5′-O-dimethoxytrityl-5-methyluridine, 3′-O-acetyl-2′-O-methoxy-ethyl-5′-O-dimethoxytrityl-5-methyluridine, 3′-O-acetyl-2′-O-methoxyethyl-5′-O-dimethoxytrityl-5-methyl-4-triazoleuridine, 2′-O-methoxyethyl-5′-O-dimethoxytrityl-5-methylcytidine, N4-benzoyl-2′-O-methoxyethyl-5′-O-dimethoxytrityl-5-methylcytidine, and N4-benzoyl-2′-O-methoxyethyl-5′-O-di-methoxytrityl-5-methylcytidine-3′-amidite; 2′-O-(aminooxyethyl) nucleoside amidites and 2′-O-(dimethylaminooxyethyl) nucleoside amidites such as 2′-(dimethylaminooxyethoxy) nucleoside amidites, 5′-O-tert-butyldiphenylsilyl-O²-2′-anhydro-5-methyluridine, 5′-O-tert-butyl-diphenylsilyl-2′-O-(2-hydroxyethyl)-5-methyluridine, 2′-O-([2-phthalimidoxy)ethyl]-5′-t-butyldiphenyl-silyl-5-methyl-uridine, 5′-O-tert-butyldiphenylsilyl-2′-O-[(2-formadoximinooxy)ethyl]-5-methyluridine, 5′-O-tert-butyldiphenylsilyl-2′-O—[N,N-dimethylaminooxyethyl]-5-methyluridine, 2′-O-(dimethylaminooxy-ethyl)-5-methyluridine, 5′-O-DMT-2′-O-(dimethylaminooxyethyl)-5-methyluridine, and 5′-O-DMT-2′-O-(2-N,N-dimethylaminooxyethyl)-5-methyluridine-3′-[(2-cyanoethyl)-N,N-diisopropylphosphor-amidite]; and 2′-(aminooxyethoxy) nucleoside amidites such as N2-isobutyryl-6-O-diphenyl-carbamoyl-2′-O-(2-ethylacetyl)-5′-O-(4,4′-dimethoxytrityl)guanosine-3′-[(2-cyanoethyl)-N,N-diiso-propylphosphoramidite].

Modified oligonucleosides may also be used in oligonucleotide synthesis, for example methylenemethylimino-linked oligonucleosides, also called MMI-linked oligonucleosides; methylene-dimethylhydrazo-linked oligonucleosides, also called MDH-linked oligonucleosides; methylene-carbonylamino-linked oligonucleosides, also called amide-3-linked oligonucleosides; and methylene-aminocarbonyl-linked oligonucleosides, also called amide-4-linked oligonucleosides, as well as mixed backbone compounds having, for instance, alternating MMI and P═O or P═S linkages, which are prepared as described in U.S. Pat. Nos. 5,378,825, 5,386,023, 5,489,677, 5,602,240 and 5,610,289. Formacetal- and thioformacetal-linked oligonucleosides may also be used and are prepared as described in U.S. Pat. Nos. 5,264,562 and 5,264,564; and ethylene oxide linked oligonucleosides may also be used and are prepared as described in U.S. Pat. No. 5,223,618. Peptide nucleic acids (PNAs) may be used as in the same manner as the oligonucleotides described above, and are prepared in accordance with any of the various procedures referred to in Peptide Nucleic Acids (PNA): Synthesis, Properties and Potential Applications, Bioorganic & Medicinal Chemistry, 1996, 4, 5-23; and U.S. Pat. Nos. 5,539,082, 5,700,922, and 5,719,262.

Chimeric oligonucleotides, oligonucleosides, or mixed oligonucleotides/oligonucleosides of the invention can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”. Some examples of different types of chimeric oligonucleotides are: [2′-O-Me]-[2′-deoxy]-[2′-O-Me] chimeric phosphorothioate oligonucleotides, [2′-O-(2-methoxyethyl)]-[2′-deoxy]-[2′-O-(methoxyethyl)] chimeric phosphorothioate oligonucleotides, and [2′-O-(2-methoxy-ethyl)phosphodiester]-[2′-deoxy phosphoro-thioate]-[2′-O-(2-methoxyethyl)phosphodiester] chimeric oligonucleotides, all of which may be prepared according to U.S. Pat. No. 5,948,680. In one preferred embodiment, chimeric oligonucleotides (“gapmers”) 18 nucleotides in length are utilized, composed of a central “gap” region consisting of ten 2′-deoxynucleotides, which is flanked on both sides (5′ and 3′ directions) by four-nucleotide “wings”. The wings are composed of 2′-methoxyethyl (2′-MOE) nucleotides. The internucleoside (backbone) linkages are phosphorothioate (P═S) throughout the oligonucleotide. Cytidine residues in the 2′-MOE wings are 5-methylcytidines. Other chimeric oligonucleotides, chimeric oligonucleosides, and mixed chimeric oligonucleo-tides/oligonucleosides are synthesized according to U.S. Pat. No. 5,623,065.

Oligonucleotides are preferably synthesized via solid phase P(III) phosphoramidite chemistry on an automated synthesizer capable of assembling 96 sequences simultaneously in a standard 96 well format. The concentration of oligonucleotide in each well is assessed by dilution of samples and UV absorption spectroscopy. The full-length integrity of the individual products is evaluated by capillary electrophoresis, and base and backbone composition is confirmed by mass analysis of the compounds utilizing electrospray-mass spectroscopy.

The effect of antisense compounds on target nucleic acid expression can be tested in any of a variety of cell types provided that the target nucleic acid is present at measurable levels. This can be routinely determined using, for example, PCR or Northern blot analysis. Cells are routinely maintained for up to 10 passages as recommended by the supplier. When cells reached 80% to 90% confluency, they are treated with oligonucleotide. For cells grown in 96-well plates, wells are washed once with 200 microliters OPTI-MEM-1 reduced-serum medium (Gibco BRL) and then treated with 130 microliters of OPTI-MEM-1 containing 3.75 g/mL LIPOFECTIN (Gibco BRL) and the desired oligonucleotide at a final concentration of 150 nM. After 4 hours of treatment, the medium is replaced with fresh medium. Cells are harvested 16 hours after oligonucleotide treatment. Preferably, the effect of several different oligonucleotides should be tested simultaneously, where the oligonucleotides hybridize to different portions of the target nucleic acid molecules, in order to identify the oligonucleotides producing the greatest degree of inhibition of expression of the target nucleic acid.

Antisense modulation of TACE nucleic acid expression can be assayed in a variety of ways known in the art. For example, TACE mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+mRNA. Methods of RNA isolation and Northern blot analysis are taught in, for example, Ausubel, F. M. et al., Current Protocols in Molecular Biology, Volume 1, pp. 4.1.1-4.2.9 and 4.5.1-4.5.3, John Wiley & Sons, Inc., 1996. Real-time quantitative (PCR) can be conveniently accomplished using the commercially available ABI PRISM 7700 Sequence Detection System, available from PE-Applied Biosystems, Foster City, Calif. and used according to manufacturer's instructions. This fluorescence detection system allows high-throughput quantitation of PCR products. As opposed to standard PCR, in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., JOE or FAM, obtained from either Operon Technologies Inc., Alameda, Calif. or PE-Applied Biosystems, Foster City, Calif.) is attached to the 5′ end of the probe and a quencher dye (e.g., TAMRA, obtained from either Operon Technologies Inc., Alameda, Calif. or PE-Applied Biosystems, Foster City, Calif.) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular (six-second) intervals by laser optics built into the ABI PRISM 7700 Sequence Detection System. In each assay, a series of parallel reactions containing serial dilutions of mRNA from untreated control samples generates a standard curve that is used to quantitate the percent inhibition after antisense oligonucleotide treatment of test samples. Other methods of quantitative PCR analysis are also known in the art. TACE protein levels can be quantitated in a variety of ways well known in the art, such as immunoprecipitation, Western blot analysis (immunoblotting), ELISA, or fluorescence-activated cell sorting (FACS). Antibodies directed to TACE polypeptides can be prepared via conventional antibody generation methods such as those described herein. Immunoprecipitation methods, Western blot (immunoblot) analysis, and enzyme-linked immunosorbent assays (ELISA) are standard in the art (see, for example, Ausubel, F. M. et al., Current Protocols in Molecular Biology, Volume 2, pp. 10.16.1-10.16.11, 10.8.1-10.8.21, and 11.2.1-11.2.22, John Wiley & Sons, Inc., 1991).

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### SEQUENCES PRESENTED IN THE SEQUENCE LISTING

| SEQ ID NO | Type | Description |
|---|---|---|
| SEQ ID NO: 1 | Amino acid | Human TACE (SWISSPROT accession number P78536) |
| SEQ ID NO: 2 | Amino acid | Human TACE variant (GenBank accession number XP_015606) |
| SEQ ID NO: 3 | Amino acid | Mus musculus LDLr (GenBank accession number I48623) |
| SEQ ID NO: 4 | Amino acid | Mus musculus LR11/SorLA (SWISSPROT accession number |
| | | O88307) |
| SEQ ID NO: 5 | Amino acid | Mus musculus AXLr; AXL receptor tyrosine kinase (GenBank |
| | | accession number NP_033491) |
| SEQ ID NO: 6 | Amino acid | Mus musculus SHPS-1; SHP substrate 1 (GenBank accession number |
| | | JC5289) |
| SEQ ID NO: 7 | Amino acid | Mus musculus Jagged1 (GenBank accession number NP_038850) |
| SEQ ID NO: 8 | Amino acid | Mus musculus ICOSL (GenBank accession number NP_056605) |
| SEQ ID NO: 9 | Amino acid | Mus musculus ICOSL splice variant “GL50-B” (GenBank accession |
| | | number AAK77544) |
| SEQ ID NO: 10 | Amino acid | Mus musculus CD14 antigen (GenBank accession number |
| | | NP_033971) |
| SEQ ID NO: 11 | Amino acid | Mus musculus CD18 antigen (GenBank accession number S04847) |
| SEQ ID NO: 12 | Amino acid | Mus musculus TEM7R; tumor endothelial marker 7-related |
| | | (GenBank accession number AAL11998) |

What is claimed is:

1. A method for identifying metalloprotease antagonists, comprising the steps of
(a) contacting cells with a compound; and
(b) measuring the amount of protein shed by the cells in the presence and in the absence of the compound;
wherein the protein is selected from the group consisting of LDLr, SHPS-1, LR11/SorLA, AXLr, Jagged1, ICOSL, CD14, CD18, and TEM7R; and wherein the compound is a metalloprotease antagonist if its presence decreases the amount of protein shed by cells.

2. A method for identifying metalloprotease agonists, comprising the steps of
(a) contacting cells with a compound; and
(b) measuring the amount of protein shed by the cells in the presence and in the absence of the compound;
wherein the protein is selected from the group consisting of LDLr, SHPS-1, LR11/SorLA, AXLr, Jagged1, ICOSL, CD14, CD18, and TEM7R; and wherein the compound is a metalloprotease agonist if its presence increases the amount of protein shed by cells.

3. The method of claim 1 wherein the protein is selected from the group consisting of LDLr, LR11/SorLA, and AXLr, and the metalloprotease is TACE.

4. The method of claim 2 wherein the protein is selected from the group consisting of LDLr, LR11/SorLA, and AXLr, and the metalloprotease is TACE.

5. The method of claim 1 wherein the amount of protein shed by the cells is measured using one or more antibodies that specifically bind to the extracellular domain of the protein.

6. The method of claim 2 wherein the amount of protein shed by the cells is measured using one or more antibodies that specifically bind to the extracellular domain of the protein.

7. A method for the treatment of a disease characterized by disruption of LDLr lipoprotein transport activity comprising administering to a mammalian subject an effective amount of a TACE inhibitor.

8. The method of claim 7, wherein said mammalian subject is human.

9. The method of claim 7, wherein said disease is selected from the group consisting of familial hypercholesterolemia, atherosclerosis, dyslipidemia, aortic aneurisms; arteritis; vascular occlusion, including cerebral artery occlusion; complications of coronary by-pass surgery; ischemia/reperfusion injury; myocarditis, including chronic autoimmune myocarditis and viral myocarditis; heart failure, including chronic heart failure (CHF), cachexia of heart failure; myocardial infarction; restenosis after heart surgery; silent myocardial ischemia; post-implantation complications of left ventricular assist devices; Raynaud's phenomena; thrombophlebitis; vasculitis, including Kawasaki's vasculitis; giant cell arteritis, Wegener's granulomatosis; and Schoenlein-Henoch purpura.
